(19) United States
(12) Patent Application Publication
(10) Pub. No.: US 2025/0354181 A1
(43) Pub. Date: Nov. 20, 2025

(54) MICROBIAL CELLS AND METHODS FOR PRODUCTION OF HERNANDULCIN

(71) Applicant: Danmarks Tekniske Universitet, Kongens Lyngby (DK)

(72) Inventors: Irina Borodina, Kongens Lyngby (DK); Jonathan Asmund Arnesen, Kongens Lyngby (DK); Jane Dannow Dyekjaer, Kongens Lyngby (DK)

(21) Appl. No.: 18/874,368

(22) PCT Filed: Jun. 16, 2023
(86) PCT No.: PCT/EP2023/066249

§ 371 Date: Dec. 12, 2024

(30) Foreign Application Priority Data
Jun. 16, 2022 (EP) 22179430.8

(51) Int. Cl. C12P 7/26; C12N 9/02; C12N 9/88; C12N 15/81

(52) U.S. Cl. CPC C12P 7/26; C12N 9/0042; C12N 9/0071; C12N 9/88; C12N 15/81; C12Y 106/02004; C12Y 402/03138

(57) ABSTRACT

The present invention relates to microbial production of the sweet non-calorigenic sesquiterpenoid hernandulcin. Disclosed herein are yeast cells capable of producing hernandulcin and optionally hernandulcin derivatives, said yeast cells expressing at least one (+)-epi-alpha-bisabolol synthase, at least one cytochrome P450 enzyme (CYP) and at least one cytochrome P450 reductase, preferably said CYP the Nicotiana tabacum CYP 5-epi-aristolocene dihydroxylase (NtEAH) or the Datura stramonium cytochrome P450 enzyme DsEAH.

### TECHNICAL FIELD

The present invention relates to microbial cells and in particular yeast cells for production of hemandulcin and optionally derivatives thereof in cells. The present disclosure also provides methods for producing hemandulcin in microbial cells and in particular yeast cells. Herein are also disclosed nucleic acids, expression systems and host cells for performing the present methods.

### BACKGROUND

Hemandulcin is a sweet non-calorigenic sesquiterpenoid found in the Mesoamerican plant Aztec Sweet Herb Lippia dulcis (Phyla scaberrima). Hemandulcin has been estimated to be approximately 1000 times sweeter than sucrose on a molar basis.

While hemandulcin can be extracted from L. dulcis, these extracts suffer from low yields and impurities (De Oliveira et al., 2012). However, hemandulcin itself seems to be well-tolerated by animals in animal studies and to be non-mutagenic.

Chemical synthesis of hemandulcin from (−)-isopulegol in six steps with 15% yield has been achieved (Jung et al., 2002). Recently, 182.7 mg/L hemandulcin was produced from cell suspension cultures of L. dulcis with addition of the precursor (+)-epi-α-bisabolol (Villa-Ruano et al., 2021). However, the addition of elicitors or precursors and the lack of genetic engineering tools for L. dulcis makes this approach unlikely to work for large-scale production.

Therefore, it would be advantageous to produce hemandulcin using engineerable microbes with solid track records for high terpenoid production, which could lead to higher hemandulcin production with less impurities. However, full heterologous hemandulcin production has not been possible yet, since the biosynthetic pathway for hemandulcin is not completely elucidated.

The precursor (+)-epi-α-bisabolol was shown to be produced by a sesquiterpenoid synthase from L. dulcis (LdTPS8p) (Attia et al., 2012). Expression of LdTPS8 in a pre-engineered Sacchammyces cerevisiae strain resulted in 280 mg/L (+)-epi-α-bisabolol. However, the remaining steps necessary to oxygenate (+)-epi-α-bisabolol into hemandulcin remained unresolved. In one report, co-expression of LdTPS8p and mammalian cytochromes P450 were attempted to produce hemandulcin in S. cerevisiae, but this only lead to the formation of various hydroxylated (+)-epi-α-bisabolol derivatives (Sarrade-Loucheur et al., 2020).

Thus, enzyme(s) catalysing the conversion of (+)-epi-α-bisabolol to hemandulcin need to be identified, in order to fulfil the longstanding desire to establish a microbial cell factory capable of producing hemandulcin.

### SUMMARY

The invention is as defined in the claims.

Surprisingly, the inventors have found that the cytochrome P450 enzyme Nicodana tabacum 5-epi-aristolochene dihydroxylase (NtEAH) as set forth in SEQ ID NO 2 is able to catalyse the conversion of (+)-epi-α-bisabolol to hemandulcin. The present disclosure provides microbial cells and in particular yeast cells as well as method and means for producing hemandulcin using Nicodana tabacum 5-epi-aristolochene dihydroxylase (NtEAH) as set forth in SEQ ID NO 2, or a functional variant thereof having at least 65% identity, homology or similarity thereto, capable of producing hemandulcin.

In particular, the present disclosure provides yeast cells capable of producing hemandulcin and/or derivatives thereof from (+)-epi-α-bisabolol, said yeast cells expressing a cytochrome P450 enzyme, such as the Nicotiana tabacum 5-epi-aristolochene dihydroxylase (NtEAH), the Solanum lycopersicum premnaspirodiene oxygenase-like protein SIEAH (SEQ ID NO 9), the Datura stramonium cytochrome P450 enzyme DsEAH (SEQ ID NO 11), the Capsicum chinense cytochrome P450 enzyme CcEAH (SEQ ID NO 13) or the Capsicum annuum cytochrome P450 71D7-like protein CaEAH (SEQ ID NO 15), or a functional variant thereof having at least 65% identity, homology or similarity to any of the aforementioned. Furthermore, comprised herein are yeast cells, methods and means for production of hemandulcin derivatives.

Thus, provided herein is a yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol, preferably a heterologous (+)-epi-alpha-bisabolol synthase;
ii. at least one cytochrome P450 enzyme capable of converting (+)-epi-α-bisabolol into hemandulcin, preferably a heterologous cytochrome P450 enzyme such as Nicotana tabacum 5-epi-aristolocene dihydroxylase (NtEAH) as set forth in SEQ ID NO 2, Datura stramonium DsEAH as set forth in SEQ ID NO 11, or a functional variant thereof having at least 65% identity, homology or similarity thereto; and
iii. optionally at least one cytochrome P450 reductase, preferably a heterologous cytochrome P450 reductase,
   whereby said yeast cell is capable of producing hemandulcin and/or one or more derivatives thereof.

Disclosed herein are cytochrome P450 enzymes capable of converting (+)-epi-α-bisabolol into hemandulcin, such as plant cytochrome P450 enzyme native to an organism of a genus selected from Nicotiana, Solanum, Datura, and Capsicum, such as Nicotana tabacum, Solanum lycopersicum, Datura stramonium, Capsicum chinense and C. annuum. In particular, disclosed are the plant cytochrome P450 enzymes NtEAH (SEQ ID NO 2), SIEAH (SEQ ID NO 9), DsEAH (SEQ ID NO 11), CcEAH (SEQ ID NO 13) and CaEAH (SEQ ID NO 15), or functional variants thereof having at least 65% identity, homology or similarity to any of the aforementioned.

Further provided herein is an expression system for expression in a yeast cell, said expression system comprising:
i. a nucleic acid encoding at least one (+)-epi-alpha-bisabolol synthase (EC 4.2.3.138), preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto;
ii. a nucleic acid encoding at least one cytochrome P450 enzyme, preferably a heterologous cytochrome P450 enzyme such as Nicotiana tabacum 5-epi-aristolocene dihydroxylase (NtEAH) as set forth in SEQ ID NO 2, DsEAH as set forth in SEQ ID NO 11, or a functional variant thereof having at least 65% identity, homology or similarity thereto; and
iii. optionally a nucleic acid encoding at least one cytochrome P450 reductase (EC 1.6.2.4), preferably a heterologous cytochrome P450 reductase, for example a plant cytochrome P450 reductase such as Arabidopsis thaliana cytochrome P450 reductase ATR2 (AtATR2) as set forth in SEQ ID NO 3 or a functional variant thereof having at least 70% identity, homology or similarity thereto.

Also provided herein is a method for producing hemandulcin and/or one or more derivatives thereof in a yeast cell, said method comprising the steps of:
i. providing a yeast cell disclosed herein; and
ii. incubating said yeast cell in a medium,
   whereby hemandulcin and/or derivatives thereof is produced.

Provided herein is also yeast cells comprising the above expression systems and/or nucleic acids.

Also provided herein is the use of above nucleic acids, expression systems and/or yeast cells for the production of hemandulcin and/or one or more derivatives thereof.

Also provided herein is a composition comprising hemandulcin and/or one or more derivatives thereof obtainable by a method disclosed herein.

Provided herein is also hemandulcin and/or one or more derivatives thereof obtainable by the methods disclosed herein.

Further provided herein is the use of a Nicotiana, Datura, Salanum or Capsicum cytochrome P450 enzyme in a method for producing hemandulcin and/or one or more derivatives thereof, preferably wherein the Nicotiana cytochrome P450 enzyme is Nicotiana tabacum 5-Epi-aristolochene dihydroxylase (NtEAH, SEQ ID NO 2), the Datura cytochrome P450 enzyme is the Datura stramonium cytochrome P450 enzyme DsEAH (SEQ ID NO 11), the Solanum cytochrome P450 enzyme is Solanum lycopersicum premnaspirodiene oxygenase-like protein (SIEAH, SEQ ID NO 9), the Capsicum cytochrome P450 enzyme is the Capsicum chinense cytochrome P450 enzyme CcEAH (SEQ ID NO 13) and/or the Capsicum annuum cytochrome P450 71D7-like protein (CaEAH, SEQ ID NO 15).

Provided herein is also a nucleic acid of an expression system for modifying a yeast cell, said nucleic acid or expression system comprising at least one nucleic acid encoding Nicotana tabacum 5-Epi-aristolochene dihydroxylase (NtEAH, SEQ ID NO 2), SIEAH (SEQ ID NO 9), DsEAH (SEQ ID NO 11), CcEAH (SEQ ID NO 13) or CaEAH (SEQ ID NO 15), and/or a functional variant thereof having at least 65% identity, homology or similarity to any of the aforementioned.

### DESCRIPTION OF DRAWINGS

FIG. 1: Biosynthetic pathway for the production of hemandulcin in yeast. LdTPS8, Lippia dulcis (+)-epi-α-bisabolol synthase. NtEAH, Nicotiana tabacum 5-Epi-aristolochene dihydroxylase. “CPR”, optional cytochrome P450 reductase.

FIG. 2: Mass spectra of hemandulcin peaks from authentic standard (RT: 9.33 min) and cell extracts (RT: 9.34 min) from HRN1-producer. A) MS1 spectra. B) MS2 spectra of [M+H-H2O]+.

FIG. 3: Hemandulcin production in the HRN1-producer. Averages and standard deviations are based on three or two replicates for cell pellet or supernatant, respectively. The HRN1-producer was cultivated in 2.5 mL YPD80 for 72 hours in a 24-deepwell plate.

FIG. 4: Relative abundance of hemandulcin detected in the supernatant of a control strain, the HRN1- and HRN2-producer. Data represents the results of single replicates. ST10274 was used as control strain. The strains were cultivated in 2.5 mL YPD80 for 72 hours in a 24-deepwell plate.

### DETAILED DESCRIPTION

### Definitions

(+)-epi-alpha-bisabolol synthase is a terpene synthase (TPS), it has an EC number EC 4.2.3.138, and can catalyse the reaction:

(2E,6E)-farnesyl diphosphate+H₂D<=>(+)-epi-alpha-bisabolol+diphosphate

(+)-epi-alpha-bisabolol synthase converts farnesyl diphosphate into (+)-epi-α-bisabolol. A yeast cell expressing (+)-epi-alpha-bisabolol synthase may thus be able to convert farnesyl diphosphate to (+)-epi-α-bisabolol, thus producing (+)-epi-α-bisabolol in the presence of farnesyl diphosphate.

5-epi-aristolocene dihydroxylase (EAH) is an oxidoreductase (EC 1), more specifically a cytochrome P450 enzyme (CYP). EAH is well-known for catalysing the reaction (EC 1.14.14.149):

5-epiaristolochene+2 [reduced NADPH-hemoprotein reductase]+2 O₂<=>capsidiol+2 [oxidized NADPH-hemoprotein reductase]+2 H₂O

Thus, EAH is well-known for converting 5-epiaristolochene into capsidiol, and is a recognized cytochrome P450 hydroxylase (Ralston et al., 2001). Surprisingly, the inventors have found that EAH from Nicodana tabacum (NtEAH) can also catalyse the reaction:

(+)-epi-alpha-bisabolol+2O₂+2 [reduced NADPH-hemoprotein reductase]<=>hemandulcin+3H₂O+2 [oxidized NADPH-hemoprotein reductase]

Suprisingly, the inventors have also found that DsEAH, SIEAH, CcEAH and CaEAH can catalyse the latter reaction.

Thus, in this disclosure NtEAH, DsEAH, SIEAH, CcEAH and CaEAH can convert (+)-epi-α-bisabolol into hemandulcin. A yeast cell expressing EAH may thus be able to convert (+)-epi-α-bisabolol to hemandulcin, thus producing hemandulcin in the presence of (+)-epi-α-bisabolol. EAH of the present disclosure may have an EC number belonging to EC 1.14.14.- for example EC 1.14.14.1 or another EC number belonging to or falling under EC 1.14.14.-.

Cytochrome P450 reductase (CPR) is an oxidoreductase (EC 1). CPR has an EC number EC 1.6.2.4 and can catalyse the reaction:

NADPH+n oxidized hemoprotein<=>NADP+n reduced hemoprotein

CPR converts oxidized hemoprotein into reduced hemoprotein.

Identity, homology or similarity with respect to a polynucleotide or polypeptide, are defined herein as the percentage of nucleotides or amino acids, respectively, in the candidate sequence that are identical, homologous or similar, respectively, to the residues of a corresponding native (may be codon-optimised) nucleotide or amino acid sequence, respectively, after aligning the sequences and introducing gaps, if necessary, to achieve the maximum percent identity/similarity, and considering any conservative substitutions according to the NCIUB rules ([https://iubmb.qmul.ac.uk/misc/naseq.html; NC-IUB, Eur J Biochem (1985)]) as part of the sequence identity. In particular, the percentage of similarity refers to the percentage of residues conserved with similar physiochemical properties. Neither 5′ or 3′ extensions nor insertions (for nucleic acids) or N‘ or C’ extensions nor insertions (for polypeptides) result in a reduction of identity, similarity or homology. Methods and computer programs for the alignments are well known in the art. Generally, a given identity between two sequences implies that the similarity between these sequences is at least equal to the identity; for example, if two sequences are 70% identical to one another, they cannot be less than 70% similar to one another—but could be sharing 80% similarity. Thus, throughout the present disclosure, it will be understood that any variant, such as a functional variant, or homologue said to have at least 70% identity, homology, or similarity to a specified sequence (polynucleotide or polypeptide) refers to a sequence having at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto.

Functional variant as term refers herein to functional variants of an enzyme, which retain at least some of the activity of the parent enzyme. Thus, a functional variant of a fluorinase, a phosphorylase, a nucleosidase can catalyse the same conversion as a fluorinase, a phosphorylase, or a nucleosidase, respectively, from which they are derived, although the efficiency of the conversion reaction may be different, e.g. the efficiency is decreased or increased compared to the parent enzyme or the substrate specificity is modified.

Native as term when referring to a polypeptide, such as a protein or an enzyme, or to a polynucleotide, such as a gene, coding sequence of a gene or genetic element, shall herein be construed to refer to a polypeptide or a polynucleotide which is naturally present in a wild type cell.

Heterologous as term when referring to a polypeptide, such as a protein or an enzyme, or to a polynucleotide, such as a gene, coding sequence of a gene or genetic element, shall herein be construed to refer to a polypeptide or a polynucleotide which is not naturally present in a wild type cell.

Mutation as term when used herein in the context of nucleic acid sequences refers to a change in nucleic acid sequence compared to the parent nucleic acid sequence. The term mutation covers single nucleotide mutations, but also insertions and deletions of multiple nucleotides, i.e. any change that leads to a different nucleic acid sequence than the parent nucleic acid sequence. The term mutation thus encompasses deletions, such as deletions of a whole gene or of a coding sequence of a gene, or a fragment/fraction of a gene or of a coding sequence of a gene.

Reduced activity as term may herein refer to a total or a partial loss of activity of a given polypeptide, such as a protein or an enzyme. In some cases, polypeptides are encoded by essential genes, which cannot be deleted. In these cases, activity of the polypeptide can be reduced by methods known in the art, such as downregulation of transcription or translation, or inhibition of the polypeptide. In other cases, the polypeptide is encoded by a non-essential gene, and the activity may be reduced or it may be completely lost, e.g. as a consequence of a deletion of the gene encoding the polypeptide.

Increased activity as term may herein refer to an improvement of activity of a given polypeptide, such as a protein or an enzyme. Said improvement in activity may be assessed based on the activity of the unmodified polypeptide serving as a reference. The improvement of activity may not be present at all times and/or in all conditions. Thus, the improvement of activity may be dependent on the condition wherein it is assessed such as a growth condition and therefore be considered condition-dependent However, the improvement of activity may be present at all time and/or in all conditions. Improvement of activity may be a consequence of a mutation of the gene encoding the polypeptide and/or a mutation in one or more genetic elements influencing the expression of the gene and/or the activity of the polypeptide. Improvement of activity may be achieved by modifying the promoter and/or the terminator of the gene encoding the polypeptide.

Derived from as term when referring to a polypeptide or a polynucleotide derived from an organism means that said polypeptide or polynucleotide is native to said organism, i.e. that it is naturally found in said organism.

Titer as term herein refers to the concentration of a compound or product that accumulates inside a cell and/or in the extracellular media during cultivation of the cell.

Derivative as term herein refers to any molecule, compound or product that has undergone any conversion, either obtained by means of chemicals (chemical synthesis) or catalysed by enzymes (enzymatic conversion) or a combination thereof, whereby another molecule, compound or product is being produced or synthesised.

Said produced another molecule, compound or product may be volatile or non-volatile.

With respect to a biosynthetic pathway such as a reaction series comprising at least one enzymatically catalysed reaction, a derivative refers to a molecule, compound or product that is further modified compared to any of the substrates, intermediates and/or products of said pathway. By contrast, a precursor of a given molecule, compound or product is a molecule or compound from which the given molecule, compound or product is a derivative. In other words, within a given pathway going from an initial substrate to a final product, a precursor of a molecule is typically upstream of the molecule, while a derivative of a molecule is typically obtained downstream. Thus, according to this definition a precursor of a molecule, compound or product is not a derivative of said molecule, compound or product. With respect to hemandulcin, 4-hydroxy-hemandulcin is an example of a derivative thereof, while hemandulcin is a precursor of 4-hydroxy-hemandulcin.

### Production of Hemandulcin

The present inventors have discovered that expression of Nicoiana tabacum 5-epi-aristolochene dihydroxylase NtEAH (SEQ ID NO 2), Datura stramonium cytochrome P450 enzyme DsEAH (SEQ ID NO 11), Solanum lycopersicum premnaspirodiene oxygenase-like protein SIEAH (SEQ ID NO 9), Capsicum chinense cytochrome P450 enzyme (CcEAH, SEQ ID NO 13) and/or Capsicum annuum cytochrome P450 71D7-like protein (CaEAH, SEQ ID NO 15), or functional variants thereof having at least 65% identity, homology or similarity to any of the aforementioned, and optionally at least one cytochrome P450 reductase in yeast cells capable of producing (+)-epi-α-bisabolol results in production of hemandulcin.

Thus, provided herein is a yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol, preferably a heterologous (+)-epi-alpha-bisabolol synthase such as Lippia dulcis terpene synthase 8 (LdTPS8) (EC 4.2.3.138) as set forth in SEQ ID NO 1 or a functional variant thereof having at least 70% identity, homology or similarity thereto; and
ii. at least one cytochrome P450 enzyme capable of converting (+)-epi-α-bisabolol into hemandulcin, preferably a heterologous cytochrome P450 enzyme such as Nicotana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto,
   whereby said yeast cell is capable of producing hemandulcin and/or one or more derivatives thereof.

Also provided herein is a yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol, preferably a heterologous (+)-epi-alpha-bisabolol synthase such as Lippia dulcis terpene synthase 8 (LdTPS8) (EC 4.2.3.138) as set forth in SEQ ID NO 1 or a functional variant thereof having at least 70% identity, homology or similarity thereto;
   ii. at least one cytochrome P450 enzyme capable of converting (+)-epi-α-bisabolol into hemandulcin, preferably a heterologous cytochrome P450 enzyme such as Nicodana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto; and
   iii. optionally at least one cytochrome P450 reductase (EC 1.6.2.4), preferably a heterologous cytochrome P450 reductase such as Arabidopsis thaliana cytochrome P450 reductase ATR2 (AtATR2) as set forth in SEQ ID NO 3 or a functional variant thereof having at least 70% identity, homology or similarity thereto,
      whereby said yeast cell is capable of producing hemandulcin and/or one or more derivatives thereof.

### (+)-Epi-Alpha-Bisabolol Synthase (TPS)

(+)-epi-alpha-bisabolol synthase is a terpene synthase (TPS) with EC 4.2.3.138 that can convert farnesyl diphosphate (FPP) to (+)-epi-alpha-bisabolol. Thus, in the present disclosure (+)-epi-alpha-bisabolol synthase will sometimes be referred to as (+)-epi-α-bisabolol synthase, TPS8 or TPS8p and the names will be used interchangeably. (+)-epi-alpha-bisabolol synthase may also in the field be referred to as EAS or EASp. In this disclosure, (+)-epi-alpha-bisabolol may also be referred to as (+)-epi-α-bisabolol and the names will be used interchangeably herein.

The (+)-epi-alpha-bisabolol synthase preferably originates from the organism Lippia dulcis. The gene encoding the (+)-epi-alpha-bisabolol synthase such as LpTPS8 may be codon-optimized for the yeast cell expressing the (+)-epi-alpha-bisabolol synthase, as is known in the art.

Conversion of FPP to (+)-epi-alpha-bisabolol can be detected using Gas Chromatography (GC) coupled to Mass Spectrometry (MS) or GC coupled to a Flame Ionization Detector (FID) as described for example by Attia et al., 2012.

### Cytochrome P450 Enzyme (CYP)

In particular the present disclosure relates to cytochrome P450 enzymes (CYPs) capable of converting (+)-epi-alpha-bisabolol into hemandulcin as well as yeast cells and methods for production of hemandulcin by expression and/or use of said CYPs. Said CYPs may also be referred to as “(+)-epi-alpha-bisabolol oxygenases”, a “(+)-epi-alpha-bisabolol dihydroxylases” or “at least one cytochrome P450 enzyme”.

Thus, provided herein is a yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol;
ii. at least one cytochrome P450 enzyme capable of converting (+)-epi-α-bisabolol into hemandulcin, preferably a heterologous cytochrome P450 enzyme such as a heterologous plant cytochrome P450 enzyme; and
iii. optionally at least one cytochrome P450 reductase (EC 1.6.2.4),
   whereby said yeast cell is capable of producing hemandulcin and/or one or more derivatives thereof.

Conversion of (+)-epi-alpha-bisabolol to hemandulcin can be detected using Liquid Chromatography coupled to MS as described for example by De Oliveira et al., 2012, or as in the examples of this disclosure.

### 5-Epi-Aristolochene Dihydroxylase (EAH)

5-epi-aristolocene dihydroxylase (EAH) is a cytochrome P450 enzyme (CYP). In the present disclosure, 5-epi-aristolochene dihydroxylase will sometimes also be referred to as EAH, EAHp, and the names will be used interchangeably.

EAH is well-known for converting 5-epiaristolochene (sometimes 5-epi-aristolochene) into capsidiol (Ralston et al., 2001). Interestingly, the inventors have found that EAH from Nicotiana tabacum (NtEAH) can catalyse the reaction:

(+)-epi-alpha-bisabolol+2O₂+2 [reduced NADPH-hemoprotein reductase]<=>hemandulcin+3H₂O+2 [oxidized NADPH-hemoprotein reductase]

Thus, NtEAH may be considered a cytochrome P450 oxygenase, such as a (+)-epi-alpha-bisabolol oxygenase or a (+)-epi-alpha-bisabolol dihydroxylase. The EAH preferably originates from the organism Nicotiana tabacum. Nicotiana tabacum 5-epi-aristolochene dihydroxylase may be referred to as NtEAH or NtEAHp herein, and the names will be used interchangeably. NtEAH may be encoded by the nucleic acid set forth in SEQ ID NO 5 (NtEAH). The gene encoding EAH such as NtEAH may be codon-optimized for the yeast cell expressing the EAH, as is known in the art.

### EAH-Related CYPs

In addition to NtEAH, further CYPs capable of converting (+)-epi-alpha-bisabolol into hemandulcin are disclosed herein. Said CYPs may be referred to as EAH-related CYPs. In particular CYPs from plants are provided herein. The provided CYPs of the present disclosure can catalyse the reaction

(+)-epi-alpha-bisabolol+2O₂+2 [reduced NADPH-hemoprotein reductase]<=>hemandulcin+3H₂O+2 [oxidized NADPH-hemoprotein reductase]

Thus, the cytochrome P450 enzyme disclosed herein, such as the at least one cytochrome P450 enzyme or the at least one plant cytochrome P450 enzyme, may be a CYP native to an organism of a genus selected from Nicotiana, Solanum, Datura, and Capsicum, such as Nicotiana tabacum, Solanum lycopersicum, Datura stramonium, Capsicum chinense and C. annuum. Preferably, said cytochrome P450 enzyme is a plant cytochrome P450 enzyme, such as NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned.

Thus, in some embodiments the at least one plant cytochrome P450 enzyme is a Datura cytochrome P450 enzyme, preferably a Datura stramonium cytochrome P450 enzyme, more preferably DsEAH as set forth in SEQ ID NO 11, or a functional variant thereof having at least 65% identity, homology or similarity thereto.

In some further embodiments the at least one plant cytochrome P450 enzyme is a premnaspirodiene oxygenase-like protein, such as a Solanum premnaspirodiene oxygenase-like protein, preferably a Solanum lycopersicum premnaspirodiene oxygenase-like protein, more preferably the Solanum lycopersicum premnaspirodiene oxygenase-like protein (SIEAH) as set forth in SEQ ID NO 9 or a functional variant thereof having at least 65% identity, homology or similarity thereto.

In other embodiments the at least one plant cytochrome P450 enzyme is a cytochrome P450 71D7-like protein, such as a Capsicum cytochrome P450 71D7-like protein, preferably a Capsicum annuum cytochrome P450 71D7-like protein, more preferably the Capsicum annuum cytochrome P450 71D7-like protein (CaEAH) as set forth in SEQ ID NO 15 or a functional variant thereof having at least 65% identity, homology or similarity thereto.

In further other embodiments the at least one plant cytochrome P450 enzyme is a Capsicum cytochrome P450 enzyme, preferably a Capsicum chinense cytochrome P450 enzyme, more preferably the CcEAH as set forth in SEQ ID NO 13 or a functional variant thereof having at least 65% identity, homology or similarity thereto.

### Cytochrome P450 Reductase

CYPs may be dependent on reductases such as CPRs that facilitate electron transfer between NAD(P)H and CYP, a so-called redox reaction. CPRs of EC 1.6.2.4 can convert oxidized hemoprotein to reduced hemoprotein. It may thus be necessary for the yeast cell to also express a CPR to assist the CYP. In some embodiments the yeast cell thus expresses, in addition to at least one (+)-epi-alpha-bisabolol synthase and the at least one cytochrome P450 enzyme (CYP), a CPR, preferably a heterologous CPR. In some embodiments, the at least one further CPR is a plant CPR. The reductase assisting the CYP such as NtEAH (SEQ ID NO 2), DsEAH (SEQ ID NO 11), SI (SEQ ID NO 9), CcEAH (SEQ ID NO 13) or CaEAH (SEQ ID NO 15), or a functional variant thereof having at least 65% identity, homology or similarity thereto may be any reductase capable of assisting the CYP such as a CPR of EC 1.6.2.4, but is not limited hereto.

Many CPRs, such as plant CPRs, are known to be promiscuous and capable of assisting multiple different CYPs. Importantly, the CPR is capable of assiting the CYP catalysing the reaction:

(+)-epi-alpha-bisabolol+2O₂+2 [reduced NADPH-hemoprotein reductase]<=>hemandulcin+3H₂O+2 [oxidized NADPH-hemoprotein reductase]

Thus, the person skilled in the art is well capable of identifying a CPR that is capable of assisting the CYP that is capable of converting (+)-epi-α-bisabolol to hemandulcin, such as NtEAH (SEQ ID NO 2), DsEAH (SEQ ID NO 11), SI (SEQ ID NO 9), CcEAH (SEQ ID NO 13) or CaEAH (SEQ ID NO 15). The CPR preferably originates from a plant such as Lippia dulcis, Arabidopsis thaliana, Medicago truncatula or Glycyrrhiza uralensis. Other CPRs that are capable of assisting CYPs in catalysing a redox reaction of interest when expressed in yeast may be identified by the person skilled in the art such as in Istiandari et al. 2021. The gene encoding the CPR may be codon-optimized for the yeast cell expressing the CPR, as is known in the art.

In some embodiments, the at least one CPR and/or the at least one further CPR are heterologous CPRs, optionally plant CPRs. In some embodiments, said CPR and/or plant CPR is selected from a Lippia dulcis CPR such as LdCPR1 (SEQ ID NO 7), a Arabidopsis thaliana CPR such as AtATR2 (NP_194750.1) or AtATR1 (NP_194183.1), a Medicago truncatula CPR such as MtCPR1 (XP_003602898.1) or MtCPR2 (XP 003610109.1), a Lotus japonicas CPR such as LjCPR1 or LjCPR2 (BAG68945.1), or Glycyrrhiza uralensis CPR such as GuCPR1 (AUG98241.1) or GuCPR2 (QCZ35624.1), or a functional variant thereof having at least 70% identity, homology or similarity thereto, encoding a CPR.

In some embodiments, the at least one further CPR is a Lippia CPR. In some embodiments the CPR is a Lippia dulcis CPR such as the Lippia dulcis CPR LdCPR1 as set forth in SEQ ID NO 7, or a functional variant thereof having at least 70% identity, homology or similarity thereto. LdCPR1 may have an EC number EC 1.6.2.4.

### Yeast Cell

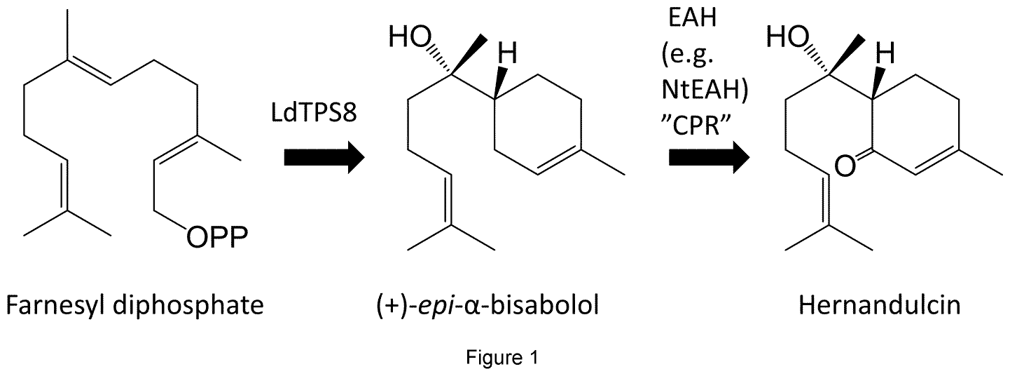

The present disclosure relates to a yeast cell which has been modified or engineered to be capable of producing (+)-epi-α-bisabolol, hemandulcin and/or derivatives thereof, for example as outlined in FIG. 1. The inventors have found that expression of a (+)-epi-alpha-bisabolol synthase, a cytochrome P450 enzyme such as 5-epiaistolochene 1,3-dihydroxylase and optionally a cytochrome P450 reductase in a yeast cell results in production of hemandulcin. Derivatives of hemandulcin may also be obtained by expressing additional enzymes in the yeast cell. The yeast cell may be engineered as disclosed herein; the nature of the desired product and/or titers will dictate which enzymes should be introduced in the yeast cell and/or how it should be modified.

In one aspect, the present disclosure provides a yeast cell capable of producing hemandulcin and/or one or more derivatives thereof, said yeast cell expressing:
at least one (+)-epi-alpha-bisabolol synthase, preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto,
wherein the at least one (+)-epi-alpha-bisabolol synthase is capable of converting farnesyl diphosphate (FPP) into (+)-epi-α-bisabolol;
at least one cytochrome P450 enzyme capable of converting (+)-epi-α-bisabolol into hemandulcin, preferably a heterologous cytochrome P450 enzyme such as Nicodana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity to any of the aforementioned; and
optionally at least one cytochrome P450 reductase, preferably a heterologous cytochrome P450 reductase such as Arabidopsis thaliana cytochrome P450 reductase ATR2 (AtATR2) as set forth in SEQ ID NO 3 or a functional variant thereof having at least 70% identity, homology or similarity thereto,
wherein the at least one cytochrome P450 enzyme can catalyse the conversion of (+)-epi-α-bisabolol into hemandulcin,
   whereby said yeast cell is capable of producing hemandulcin and/or one or more derivatives thereof.

In some embodiments, the yeast cell expresses at least one further cytochrome P450 reductase (CPR), preferably a heterologous CPR. In preferred embodiments, said at least one further CPR is a CPR with EC number EC 1.6.2.4. In some embodiments, said at least one further CPR is a plant CPR such as a Lippia CPR, such as a Lippia dulcis CPR. In some embodiments, the L. dulcis CPR LdCPR1 as set forth in SEQ ID NO 7, or a functional variant thereof having at least 70% identity, homology or similarity thereto.

In some embodiments, the yeast cell is a Saccharomyces cerevisiae cell or a Yarrowia lipolytica cell.

Throughout the present disclosure, it will be understood that the yeast cell can produce the compounds of interest listed herein when incubated in a cultivation medium under conditions that enable the yeast cell to grow and produce the desired compound. From the description of the yeast cells provided herein, the skilled person will not have difficulties in identifying suitable cultivation media and conditions to achieve production.

### Production of Hemandulcin

The yeast cell of the present disclosure can produce hemandulcin. This requires that the yeast cell expresses at least one TPS, such as at least one (+)-epi-alpha-bisabolol synthase, capable of converting farnesyl diphosphate (FPP) to (+)-epi-α-bisabolol, and at least one cytochrome P450 enzyme and optionally at least one cytochrome P450 reductase (CPR) for conversion of (+)-epi-α-bisabolol to hemandulcin. In some embodiments, the yeast cell expresses at least one further CPR.

In some embodiments, the at least one TPS is a heterologous TPS. In some embodiments the TPS and/or the heterologous TPS is a plant TPS. In some embodiments, the TPS is a (+)-epi-alpha-bisabolol synthase (EC 4.2.3.138).

In preferred embodiments, the at least one TPS is (+)-epi-alpha-bisabolol synthase (EC 4.2.3.138). In some embodiments, the (+)-epi-alpha-bisabolol synthase is a heterologous (+)-epi-alpha-bisabolol synthase. In some embodiments the (+)-epi-alpha-bisabolol synthase is a Lippia (+)-epi-alpha-bisabolol synthase, such as a Lippia dulcis (+)-epi-alpha-bisabolol synthase. In some embodiments, the Lippia dulcis (+)-epi-alpha-bisabolol synthase is Lippia dulcis terpene synthase 8 (LpTPS8) as set forth in SEQ ID NO 1 or a functional variant thereof having at least 70% identity, homology or similarity thereto, wherein LpTPS8 is capable of converting farnesyl diphosphate to (+)-epi-α-bisabolol.

In preferred embodiments, the at least one cytochrome P450 enzyme (CYP) capable of converting (+)-epi-α-bisabolol into hemandulcin is a heterologous CYP. Said CYP may also be referred to as (+)-epi-alpha-bisabolol oxygenases, or (+)-epi-alpha-bisabolol dihydroxylases. In some embodiments, the CYP is a Nicotiana CYP. In other embodiments, the CYP is a CYP native to an organism of a genus selected from Nicotiana, Solanum, Datura, and Capsicum. In some embodiments, the CYP is a cytochrome P450 oxygenase. In some embodiments, the CYP is a heterologous cytochrome P450 oxygenase.

In some embodiments, the CYP is a cytochrome P450 oxygenase from Nicotiana such as from Nicotiana tabacum. In some embodiments, the CYP is a 5-epi-aristolocene dihydroxylase (EAH). In some embodiments, the CYP is a Nicotiana EAH. In some embodiments, the CYP is a Nicotiana tabacum 5-epi-aristolocene dihydroxylase (NtEAH). In preferred embodiments the Nicotiana tabacum 5-epi-aristolocene dihydroxylase (NtEAH) is the Nicotiana tabacum 5-epi-aristolocene dihydroxylase (NtEAH) as set forth in SEQ ID NO 2 or a functional variant thereof having at least 65% identity, homology or similarity thereto, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto, wherein NtEAH is capable of converting (+)-epi-α-bisabolol into hemandulcin.

In some embodiments the CYP is a cytochrome P450 enzyme from Capsicum such as from Capsicum annuum. In some embodiments, the CYP is a cytochrome P450 71D7-like protein. In other embodiments, the CYP is a Capsicum cytochrome P450 71D7-like protein. In some embodiments, the CYP is a Capsicum annuum cytochrome P450 71D7-like protein capable of converting (+)-epi-α-bisabolol into hemandulcin. In preferred embodiments, the Capsicum annuum cytochrome P450 71D7-like protein is the Capsicum annuum cytochrome P450 71D7-like protein (CaEAH) as set forth in SEQ ID NO 15 or a functional variant thereof having at least 65% identity, homology or similarity thereto, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto.

In some embodiments the CYP is a cytochrome P450 enzyme from Solanum such as from Solanum lycopersicum. In some embodiments, the CYP is a premnaspirodiene oxygenase-like protein. In other embodiments, the CYP is a Solanum premnaspirodiene oxygenase-like protein. In some embodiments, the CYP is a Solanum lycopersicum premnaspirodiene oxygenase-like protein capable of converting (+)-epi-α-bisabolol into hemandulcin. In preferred embodiments, the Solanum lycopersicum premnaspirodiene oxygenase-like protein is the Solanum lycopersicum premnaspirodiene oxygenase-like protein (SIEAH) as set forth in SEQ ID NO 9 or a functional variant thereof having at least 65% identity, homology or similarity thereto, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto.

In other embodiments, the CYP is a cytochrome P450 enzyme from Datura such as Datura stramonium. In some embodiments the CYP is a Datura stramonium cytochrome P450 enzyme capable of converting (+)-epi-α-bisabolol to hemandulcin. In preferred embodiments the Datura stramonium cytochrome P450 enzyme is DsEAH as set forth in SEQ ID NO 11, or a functional variant thereof having at least 65% identity, homology or similarity identity, homology or similarity thereto, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto.

In other embodiments, the CYP is a cytochrome P450 enzyme from Capsicum such as Capsicum chinense. In some embodiments the CYP is a Capsicum chinense cytochrome P450 enzyme capable of converting (+)-epi-α-bisabolol to hemandulcin. In preferred embodiments the Capsicum chinense cytochrome P450 enzyme is CcEAH as set forth in SEQ ID NO 13, or a functional variant thereof having at least 65% identity, homology or similarity identity, homology or similarity thereto, such as at least 66%, such as at least 67%, such as at least 68%, such as at least 69%, such as at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology thereto.

Thus, in another aspect, the present disclosure provides a yeast cell capable of producing hemandulcin and/or one or more derivatives thereof, said yeast cell expressing:
at least one (+)-epi-alpha-bisabolol synthase, preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto,
wherein the at least one (+)-epi-alpha-bisabolol synthase is capable of converting farnesyl diphosphate (FPP) into (+)-epi-α-bisabolol; and
at least one cytochrome P450 enzyme capable of converting (+)-epi-α-bisabolol into hemandulcin, preferably a heterologous cytochrome P450 enzyme such as Nicotiana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto,
wherein the at least one cytochrome P450 enzyme can catalyse the conversion of (+)-epi-α-bisabolol into hemandulcin,
   whereby said yeast cell is capable of producing hemandulcin and/or one or more derivatives thereof.

In some embodiments, the yeast cell further expresses at least one cytochrome P450 reductase. In some embodiments, the at least one cytochrome P450 reductase (CPR) is a heterologous CPR, optionally with EC number EC 1.6.2.4. In other embodiments, the at least one CPR is a CPR with EC number EC 1.6.2.4. In some embodiments, the CPR is a plant CPR. In some embodiments, the CPR is an Arabidopsis CPR. In some embodiments, the CPR is an Arabidopsis thaliana CPR such as the Arabidopsis thaliana CPR ATR2 (AtATR2) (EC 1.6.2.4) as set forth in SEQ ID NO 3 or a functional variant thereof having at least 70% identity, homology or similarity thereto. In some embodiments, the at least one CPR is a reductase such as an oxidoreductase or another reductase capable of assisting the CYP in the conversion of (+)-epi-α-bisabolol into hemandulcin by converting oxidized hemoprotein to reduced hemoprotein.

In some embodiments, the at least one CPR and/or the at least one further CPR is the same. In some embodiments, the optionally at least one CPR and/or the at least one further CPR are different.

In one aspect, the present disclosure provides a yeast cell capable of producing hemandulcin and/or one or more derivatives thereof, said yeast cell expressing:
at least one (+)-epi-alpha-bisabolol synthase, preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto,
wherein the at least one (+)-epi-alpha-bisabolol synthase is capable of converting farnesyl diphosphate (FPP) into (+)-epi-α-bisabolol;
at least one cytochrome P450 enzyme capable of converting (+)-epi-α-bisabolol into hemandulcin, preferably a heterologous cytochrome P450 enzyme such as Nicotiana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto; and
at least one cytochrome P450 reductase, preferably a heterologous cytochrome P450 reductase such as Arabidopsis thaliana cytochrome P450 reductase ATR2 (AtATR2) as set forth in SEQ ID NO 3 or a functional variant thereof having at least 70% identity, homology or similarity thereto,
wherein the at least one cytochrome P450 enzyme can catalyse the conversion of (+)-epi-α-bisabolol into hemandulcin,
   whereby said yeast cell is capable of producing hemandulcin and/or one or more derivatives thereof.

In some embodiments, the at least one further CPR is a heterologous CPR. In some embodiments, the at least one further CPR is a plant CPR. In preferred embodiments the at least one further CPR has an EC number EC 1.6.2.4. In some embodiments, the at least one further CPR is a Lippia CPR. In some embodiments the CPR is a Lippia dulcis CPR such as the Lippia dulcis CPR LdCPR1 as set forth in SEQ ID NO 7, or a functional variant thereof having at least 70% identity, homology or similarity thereto. LdCPR1 may have an EC number EC 1.6.2.4.

In some embodiments, the at least one CPR and/or the at least one further CPR are heterologous CPRs, optionally plant CPRs. In some embodiments, said plant CPR is selected from a Lippia dulcis CPR such as LdCPR1 (SEQ ID NO 7), a Arabidopsis thaliana CPR such as AtATR2 (NP 194750.1) or AtATR1 (NP_194183.1), a Medicago truncatula CPR such as MtCPR1 (XP_003602898.1) or MtCPR2 (XP_003610109.1), a Lotus japonicas CPR such as LjCPR1 or LjCPR2 (BAG68945.1), or Glycyrrhiza uralensis CPR such as GuCPR1 (AUG98241.1) or GuCPR2 (QCZ35624.1), or a functional variant thereof having at least 70% identity, homology or similarity thereto, encoding a CPR.

In further another aspect, the present disclosure provides a yeast cell capable of producing hemandulcin and/or one or more derivatives thereof, said yeast cell expressing:
at least one (+)-epi-alpha-bisabolol synthase, preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto,
wherein the at least one (+)-epi-alpha-bisabolol synthase is capable of converting farnesyl diphosphate (FPP) into (+)-epi-α-bisabolol;
at least one cytochrome P450 enzyme capable of converting (+)-epi-α-bisabolol into hemandulcin, preferably a heterologous cytochrome P450 enzyme such as Nicofiana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto;
at least one cytochrome P450 reductase, preferably a heterologous cytochrome P450 reductase such as Arabidopsis thaliana cytochrome P450 reductase ATR2 (AtATR2) as set forth in SEQ ID NO 3 or a functional variant thereof having at least 70% identity, homology or similarity thereto; and
at least one further CPR, preferably a heterologous CPR, such as the Lippia dulcis CPR LdCPR1 as set forth in SEQ ID NO 7, or a functional variant thereof having at least 70% identity, homology or similarity thereto,
wherein the at least one cytochrome P450 enzyme can catalyse the conversion of (+)-epi-α-bisabolol into hemandulcin,
   whereby said yeast cell is capable of producing hemandulcin and/or one or more derivatives thereof.

In further another aspect, the present disclosure provides a yeast cell capable of producing hemandulcin and/or one or more derivatives thereof, said yeast cell expressing:
at least one (+)-epi-alpha-bisabolol synthase, preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto,
wherein the at least one (+)-epi-alpha-bisabolol synthase is capable of converting farnesyl diphosphate (FPP) into (+)-epi-α-bisabolol;
at least one cytochrome P450 enzyme capable of converting (+)-epi-α-bisabolol into hemandulcin, preferably a heterologous cytochrome P450 enzyme such as Nicotiana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto; and
at least one further CPR, preferably a heterologous CPR, such as the Lippia dulcis CPR LdCPR1 as set forth in SEQ ID NO 7, or a functional variant thereof having at least 70% identity, homology or similarity thereto,
wherein the at least one cytochrome P450 enzyme can catalyse the conversion of (+)-epi-α-bisabolol into hemandulcin,
   whereby said yeast cell is capable of producing hemandulcin and/or one or more derivatives thereof.

In some embodiments, the yeast cell expresses at least LpTPS8 and NtEAH as set forth in SEQ ID NO 1 and SEQ ID NO 2, respectively, or functional variants thereof having at least 65% identity, homology or similarity thereto. In some other embodiments, the yeast cell expresses at least LpTPS8 and SIEAH as set forth in SEQ ID NO 1 and SEQ ID NO 9, respectively, or functional variants thereof having at least 65% identity, homology or similarity thereto. In other embodiments, the yeast cell expresses at least LpTPS8 and DsEAH as set forth in SEQ ID NO 1 and SEQ ID NO 11, respectively, or functional variants thereof having at least 65% identity, homology or similarity thereto. In other embodiments, the yeast cell expresses at least LpTPS8 and CcEAH as set forth in SEQ ID NO 1 and SEQ ID NO 13, respectively, or functional variants thereof having at least 65% identity, homology or similarity thereto. In other embodiments, the yeast cell expresses at least LpTPS8 and CaEAH as set forth in SEQ ID NO 1 and SEQ ID NO 15, respectively, or functional variants thereof having at least 65% identity, homology or similarity thereto.

In some embodiments, the yeast cell expresses at least LpTPS8, NtEAH, and AtATR2 as set forth in SEQ ID NO 1, SEQ ID NO 2 and SEQ ID NO 3, respectively, or functional variants thereof having at least 65% identity, homology or similarity thereto. In some embodiments, the yeast cell expresses at least LpTPS8, DsEAH, and AtATR2 as set forth in SEQ ID NO 1, SEQ ID NO 11 and SEQ ID NO 3, respectively, or functional variants thereof having at least 65% identity, homology or similarity thereto. In some embodiments, the yeast cell expresses at least LpTPS8, SIEAH, and AtATR2 as set forth in SEQ ID NO 1, SEQ ID NO 9 and SEQ ID NO 3, respectively, or functional variants thereof having at least 65% identity, homology or similarity thereto. In some embodiments, the yeast cell expresses at least LpTPS8, CcEAH, and AtATR2 as set forth in SEQ ID NO 1, SEQ ID NO 13 and SEQ ID NO 3, respectively, or functional variants thereof having at least 65% identity, homology or similarity thereto. In some embodiments, the yeast cell expresses at least LpTPS8, CaEAH, and AtATR2 as set forth in SEQ ID NO 1, SEQ ID NO 15 and SEQ ID NO 3, respectively, or functional variants thereof having at least 65% identity, homology or similarity thereto. In alternative embodiments, AtATR2 may be replaced by another Arabidopsis thaliana CPR such AtATR1 (NP_194183.1), a Medicago truncatula CPR such as MtCPR1 (XP_003602898.1) or MtCPR2 (XP 003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively.

In some embodiments, the yeast cell expresses at least LpTPS8, NtEAH, AtATR2, and LpCPR1 as set forth in SEQ ID NO 1, SEQ ID NO 2, SEQ ID NO 3 and SEQ ID NO 7, respectively, or functional variants thereof having at least 65% identity, homology or similarity thereto. In some embodiments, the yeast cell expresses at least LpTPS8, DsEAH, AtATR2, and LpCPR1 as set forth in SEQ ID NO 1, SEQ ID NO 11, SEQ ID NO 3 and SEQ ID NO 7, respectively, or functional variants thereof having at least 65% identity, homology or similarity thereto. In some embodiments, the yeast cell expresses at least LpTPS8, SIEAH, AtATR2, and LpCPR1 as set forth in SEQ ID NO 1, SEQ ID NO 9, SEQ ID NO 3 and SEQ ID NO 7, respectively, or functional variants thereof having at least 65% identity, homology or similarity thereto. In some embodiments, the yeast cell expresses at least LpTPS8, CcEAH, AtATR2, and LpCPR1 as set forth in SEQ ID NO 1, SEQ ID NO 13, SEQ ID NO 3 and SEQ ID NO 7, respectively, or functional variants thereof having at least 65% identity, homology or similarity thereto. In some embodiments, the yeast cell expresses at least LpTPS8, CaEAH, AtATR2, and LpCPR1 as set forth in SEQ ID NO 1, SEQ ID NO 15, SEQ ID NO 3 and SEQ ID NO 7, respectively, or functional variants thereof having at least 65% identity, homology or similarity thereto. In alternative embodiments, AtATR2 may be replaced by another Arabidopsis thaliana CPR such AtATR1 (NP_194183.1), a Medicago truncatula CPR such as MtCPR1 (XP 003602898.1) or MtCPR2 (XP 003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively.

In some embodiments, the yeast cell expresses at least LpTPS8 and NtEAH as set forth in SEQ ID NO 1 and SEQ ID NO 2, respectively, or functional variants thereof having at least 65% identity, homology or similarity thereto, and further expresses one or more of a Lippia dulcis CPR such as LdCPR1 (SEQ ID NO 7), a Arabidopsis thaliana CPR such as AtATR2 (SEQ ID NO 3) or AtATR1 (NP_194183.1), a Medicago truncatula CPR such as MtCPR1 (XP_003602898.1) or MtCPR2 (XP_003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively.

In some embodiments, the yeast cell expresses at least LpTPS8 and DsEAH as set forth in SEQ ID NO 1 and SEQ ID NO 11, respectively, or functional variants thereof having at least 65% identity, homology or similarity thereto, and further expresses one or more of a Lippia dulcis CPR such as LdCPR1 (SEQ ID NO 7), a Arabidopsis thaliana CPR such as AtATR2 (SEQ ID NO 3) or AtATR1 (NP 194183.1), a Medicago truncatula CPR such as MtCPR1 (XP_003602898.1) or MtCPR2 (XP_003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively. In some embodiments, the yeast cell expresses at least LpTPS8 and SIEAH as set forth in SEQ ID NO 1 and SEQ ID NO 9, respectively, or functional variants thereof having at least 65% identity, homology or similarity thereto, and further expresses one or more of a Lippia dulcis CPR such as LdCPR1 (SEQ ID NO 7), a Arabidopsis thaliana CPR such as AtATR2 (SEQ ID NO 3) or AtATR1 (NP 194183.1), a Medicago truncatula CPR such as MtCPR1 (XP_003602898.1) or MtCPR2 (XP 003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively. In some embodiments, the yeast cell expresses at least LpTPS8 and CcEAH as set forth in SEQ ID NO 1 and SEQ ID NO 13, respectively, or functional variants thereof having at least 65% identity, homology or similarity thereto, and further expresses one or more of a Lippia dulcis CPR such as LdCPR1 (SEQ ID NO 7), a Arabidopsis thaliana CPR such as AtATR2 (SEQ ID NO 3) or AtATR1 (NP_194183.1), a Medicago truncatula CPR such as MtCPR1 (XP 003602898.1) or MtCPR2 (XP 003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively. In some embodiments, the yeast cell expresses at least LpTPS8 and CaEAH as set forth in SEQ ID NO 1 and SEQ ID NO 15, respectively, or functional variants thereof having at least 65% identity, homology or similarity thereto, and further expresses one or more of a Lippia dulcis CPR such as LdCPR1 (SEQ ID NO 7), a Arabidopsis thaliana CPR such as AtATR2 (SEQ ID NO 3) or AtATR1 (NP 194183.1), a Medicago truncatula CPR such as MtCPR1 (XP_003602898.1) or MtCPR2 (XP_003610109.1), a Lotus japonicas CPR such as LjCPR1 or LjCPR2 (BAG68945.1), or a Glycyrrhiza uralensis CPR such as GuCPR1 (AUG98241.1) or GuCPR2 (QCZ35624.1), or a functional variant having at least 70% identity, homology or similarity to SEQ ID NO 7, SEQ ID NO 3, AtATR1, MtCPR1, MtCPR2, LjCPR1, LjCPR2, GuCPR1, or GuCPR2, respectively.

In some embodiments, the yeast cell may be further engineered to allow production of derivatives of hemandulcin. A yeast cell producing a derivative of hemandulcin may also produce hemandulcin.

### Examples of Useful Yeast Cells

The yeast cells disclosed herein producing or being capable of producing the compounds of interest, such as hemandulcin and/or one or more derivatives thereof might be referred to as production organisms, microbial factories, microbial production organisms, hosts, host cells, host organisms, production hosts, cell factories etc.

Various yeast species may be useful as production organisms of hemandulcin and/or one or more derivatives thereof according to the present disclosure. Thus, in some embodiments the yeast cell is a non-pathogenic yeast cell.

In some embodiments, the yeast cell belongs to a genus selected from Saccharomyces, Pichia, Yarrowia, Kluyveromyces, Candida, Rhodotorula, Rhodosporidium, Cryptococcus, Trichosporon and Lipomyces.

In some embodiments, the yeast cell belongs to a species selected from Saccharomyces cerevisiae, Saccharomyces boulardi, Komagetella phaffi (Pichia pastoris), Kluyveromyces marxianus, Cryptococcus albidus, Lipomyces lipofera, Lipomyces starkeyi, Rhodospondium toruloides, Rhodotorula glutinis, Trichosporon pullulan and Yarrowia lipolytica. Preferably the yeast cell is a S. cerevisiae cell or a Y. lipolytica cell. In a preferred embodiment, the yeast cell is a Y. lipolytica cell.

The yeast cell may thus be a Y. lipolytica cell expressing any of the enzymes described herein. In particular, the Y. lipolytica cell may express a TPS such as LdTPS8 (SEQ ID NO 1) and a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned. Preferably the CYP is any of NtEAH (SEQ ID NO 2) and/or DsEAH (SEQ ID NO 11).

The yeast cell may be a S. cerevisiae cell expressing any of the enzymes described herein. In particular, the S. cerevisiae cell may express a TPS such as LdTPS8 (SEQ ID NO 1) and a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned. Preferably the CYP is any of NtEAH (SEQ ID NO 2) and/or DsEAH (SEQ ID NO 11).

The yeast cell may be a cell belonging to the species of Saccharomyces boulardi, Komagetella phaffi Pichia pastoris), Kluyveromyces marxianus, Cryptococcus albidus, Lipomyces lipofera, Lipomyces starkeyi, Rhodosporidium toruloides, Rhodotorula glutinis and Trichosporon pullulan, expressing any of the enzymes described herein. In particular, the cell of Saccharomyces boulardi, Komagetella phaffi (Pichia pastoris), Kluyveromyces marxianus, Cryptococcus albidus, Lipomyces lipofera, Lipomyces starkeyi, Rhodosporidum toruloides, Rhodotorula glutinis and Trichosporon pullulan may express a TPS such as LdTPS8 (SEQ ID NO 1) and a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned. Preferably the CYP is any of NtEAH (SEQ ID NO 2) and/or DsEAH (SEQ ID NO 11).

Expression systems, nucleic acids and/or polynucleotides useful for obtaining yeast cells capable of producing hemandulcin and/or one or more derivatives thereof are disclosed anywhere herein, in particular in the section “Expression systems and nucleic acids”.

### Other Modifications

The yeast cells disclosed herein for production of hemandulcin may be further modified. Said modifications may be modifications to enhance the precursor supply, for example supply of native or heterologous metabolites involved in the production of (+)-epi-alpha-bisabolol, hemandulcin and/or derivatives thereof, or to reduce bypass-pathways. Thus, the yeast cell may be any of the yeast cells described herein above, for example a Y. lipolytica cell or a S. cerevisiae cell, and expressing a TPS, a CYP, and optionally a CPR, and may in addition be further modified as described herein below. Preferably the CYP is NtEAH (SEQ ID NO 2), SIEAH (SEQ ID NO 9), DsEAH (SEQ ID NO 11), CcEAH (SEQ ID NO 13) and/or CaEAH (SEQ ID NO 15).

Production of hernandulcin and/or one or more derivatives thereof requires farnesyl diphosphate (FPP) as a substrate. Thus, and without being bound by theory, it may require that the yeast cell is capable of producing FPP. If the yeast cell is not capable of producing FPP, it may require modification such as engineering of said yeast cell in order to enable it to synthesize FPP. Alternatively or additionally, if the yeast cell is not capable of producing FPP, production of hemandulcin and/or derivatives thereof may require supplementing FPP to the cultivation medium, whereby the yeast cell is capable of converting FPP to hemandulcin and/or one or more derivatives. Further, to this, if the yeast cell can produce FPP, production such as improved production of hemandulcin and/or derivatives thereof, may require that the production of FPP is increased such as improved.

Thus, in some embodiments, if the yeast cell is not capable of producing FPP, it may require modification or engineering of the yeast cell to enable production of FPP by said cell. It may be advantageous to modify the yeast cell in such a manner that FPP metabolism is directed towards increased FPP synthesis, thereby further increasing the titers of hernandulcin and/or one or more derivatives thereof. Thus, in some embodiments, the yeast cell comprises one or more modifications resulting in increased or improved availability of FPP. Such modification may be achieved by increasing or improving the mevalonate (MVA) pathway flux. In other words, it may be achieved by increasing the sesquiterpene pathway flux. Useful modifications is disclosed in Arnesen et al., 2020, in particular useful modifications of a Y. lipolytica cell such as one or more of the modifications of Y. lipolytica ST9149 disclosed in Arnesen et al., 2020.

In some embodiments, the one or more modifications is selected from a mutation, an insertion and/or a deletion. In some embodiments, the one or more modifications may be overexpression, upregulation, downregulation or deletion of a gene or other genetic element. In some embodiments, the one or more modifications may comprise overexpression of a gene or other genetic element.

In some embodiments, the one or more modifications may comprise modifying the activity of a polypeptide. In some embodiments, the one or more modifications may comprise reducing the activity of a polypeptide. In some embodiments, the one or more modifications may comprise increasing the activity of a polypeptide.

It may be advantageous to improve synthesis of acetyl-CoA and/or upregulate expression of one or more genes of the MVA pathway to improve synthesis of dimethylallyl diphosphate (DMAPP) and/or isopentyl diphosphate (IPP). In some embodiments, improved synthesis of DMAPP and/or IPP may comprising increasing the activity of one or more polypeptides such as proteins of the MVA pathway.

In some embodiments, the yeast cell overexpresses one or more genes involved in the synthesis of acetyl-CoA, such as overexpression of ATP citrate lyase (ACL), for example native ACL, and/or Salmonella enterica acetyl-CoA synthetase (SeACS) with a L641P substitution as described in Huang et al. 2018. In some embodiments, the yeast cell is a Y. lipolytica cell, and the cell overexpresses native ATP citrate lyase such as Y. lipolytica ATP citrate lyase 1 (YIACL1), for example YIACL1 as set forth in accession number XM_503231, or functional variants thereof having at least 70% identity, homology or similarity thereto. In other embodiments, SeACS is as set forth in accession number WP_000083882.1, with the lysine residue 641 substituted for proline (L641P) or a functional variant thereof having at least 70% identity, homology or similarity thereto. In some embodiments, the SeACS has a L641P substitution (SeACS^{L641P}). Thus, in some embodiments the SeACS is SeACS^{L641P} or functional variants thereof having at least 70% identity, homology or similarity thereto. In some embodiments, the one or more modifications comprise increasing the activity of ACL such as ACL1 or YIACL1, and/or SeACS such as SeACS^{L641P}, or functional variants thereof having at least 70% identity, homology or similarity thereto, respectively. For example, the yeast cell may be a Y. lipolytica cell expressing SeACS^{L641P}, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR and further expressing or a functional variant thereof having at least 65% identity, homology or similarity to any of the aforementioned. In other embodiments, the yeast cell may be a Y. lipolytica cell expressing YIACL1, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned. In some embodiments, the yeast cell overexpresses 3-hydroxy-3-methylglutaryl-CoA reductase (HMG), such as native HMG that can catalyse the conversion 3-hydroxy-3-methylglutaryl-CoA to mevalonic acid (mevalonate). In some embodiments, the yeast cell is a Y. lipolytica cell, and the cell overexpresses native HMG such as Y. lipolytica 3-hydroxy-3-methylglutaryl-CoA reductase 1 (YIHMG1), for example YIHMG1 as set forth in accession number XP_503558 or a functional variant thereof having at least 70% identity, homology or similarity thereto. In some embodiments, the yeast cell is S. cerevisiae, and the cell expresses or overexpress a truncated version of HMG (tHMG). In some embodiments, the one or more modifications comprise increasing the activity of HMG such as tHMG1 or YIHMG1, or functional variants thereof having at least 70% identity, homology or similarity thereto, respectively. Thus, in other embodiments, the yeast cell may be a Y. lipolytica cell overexpressing YIHMG1, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR and further expressing or a functional variant thereof having at least 65% identity, homology or similarity to any of the aforementioned. In other embodiments, the yeast cell may be a S. cerevisiae cell overexpressing tHMG1, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned.

In some embodiments, the yeast cell overexpresses mevalonate kinase, such as native mevalonate kinase, that can catalyse the conversion of mevalonic acid (mevalonate) to mevalonate-3-phosphate. In some embodiments, the yeast cell is Y. lipolytica, and the cell overexpresses native mevalonate kinase such as Y. lipolytica ERG12 (YIERG12), for example YIERG12 as set forth in accession number: XP_500956 or a functional variant thereof having at least 70% identity, homology or similarity thereto. In some embodiments, the one or more modifications comprise increasing the activity of mevalonate kinase such as YIERG12, or functional variants thereof having at least 70% identity, homology or similarity thereto, respectively. Thus, in other embodiments, the yeast cell may be a Y. lipolytica cell overexpressing YIERG12, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned.

In some embodiments, the yeast cell overexpresses isopentyl diphosphate isomerase (IDI), such as native IDI. In some embodiments, the yeast cell is Y. lipolytica, and the cell overexpresses native isopentyl diphosphate isomerase 1 such as Y. lipolytica IDI1 (YIIDI1), for example YIIDI1 as set forth in accession number XP_504974 or a functional variant thereof having at least 70% identity, homology or similarity thereto. In some embodiments, the one or more modifications comprise increasing the activity of IDI such as IDI1 or YIIDI1, or functional variants thereof having at least 70% identity, homology or similarity thereto, respectively. Thus, in other embodiments, the yeast cell may be a Y. lipolytica cell overexpressing YIIDI1, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned.

In some embodiments, the yeast cell overexpresses farnesyl diphosphate synthase (FPPS), such as ERG20. In some embodiments, the yeast cell is a Y. lipolytica cell, and the cell overexpresses native FPPS such as Y. lipolytica ERG20 (YIERG20) for example YIERG20 as set forth in accession number XP_503599 or a functional variant thereof having at least 70% identity, homology or similarity thereto. In some embodiments, the one or more modifications comprise increasing the activity of FPPS such as ERG20 or YIERG20, or functional variants thereof having at least 70% identity, homology or similarity thereto, respectively. Thus, in other embodiments, the yeast cell may be a Y. lipolytica cell overexpressing YIERG20, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned.

In some embodiments, the one or more polypeptides with modified activity, such as increased activity and/or reduced activity, are native to the yeast cell or are non-native to the yeast cell or a combination of native and non-native. In some embodiments, the mutation resulting in reduced activity of a polypeptide comprises down-regulation of a gene encoding said polypeptide and/or mutation of said polypeptide such as a loss-of-function mutation. In some embodiments, the mutation resulting in increased activity of a polypeptide comprises overexpression.

In some embodiments, the activity of native farnesyl-diphosphate farnesyl transferase, also known as squalene synthase (SQS), for example ERG9, is reduced for the yeast cell of the present disclosure as compared to the activity of SQS and/or ERG9 in a non-modified/non-engineered yeast cell. In some embodiments, reducing the activity of SQS and/or ERG9 is obtained by downregulating the expression of SQS and/or ERG9 in a modified/engineered yeast cell of the present disclosure compared to the expression in a non-modified/non-engineered cell. In some embodiments, reducing the activity of SQS and/or ERG9 is obtained by modification of the native SQS and/or ERG9 promoter (PrSQS and/or PrERG9) such as by mutating PrSQS and/or PrERG9 and/or replacing PrSQS and/or PrERG9 with the nucleic acid sequence of another promoter, for example another native or heterologous promoter to the yeast cell of the present disclosure, whereby the activity of SQS and/or ERG9 is reduced. In some embodiments, the yeast cell of the present disclosure is a Y. lipolytica cell, and the promoter of ERG9, and/or squalene synthase 1 (SQS1) (PrERG9 and/or PrSQS1) of the Y. lipolytica cell is replaced with the native promoter of lanosterol 14-alpha demethylase (ERG11) (PrERG11) of the Y. lipolytica cell. Thus, in some embodiments PrSQS such as PrSQS1 and/or PrERG9 is exchanged for PrERG11, whereby the activity of SQS and/or ERG9 is reduced. Thus, in other embodiments, the yeast cell may be a Y. lipolytica cell, wherein PrSQS1 is replaced by PrERG11, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned. Thus, in other embodiments, the yeast cell may be a Y. lipolytica cell, wherein PrERG9 is replaced by PrERG11, and further expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), and a CPR, or a functional variant thereof having at least 65% identity, homology or similarity to any of the aforementioned.

Improved production of FPP may be assessed by LC coupled to tandem mass spectrometry (MS/MS) as described in Luo et al., 2020.

The above mentioned modifications may be encoded by one or more of the nucleic acids, nucleic acid constructs, polynucleotides, nucleic acid molecules and/or expression systems disclosed in “Expression systems” herein below.

Any of the above-mentioned modifications may be introduced in the yeast cell, such as a yeast cell belonging to the species of Saccharomyces boulardi, Komagetella phaffi (Pichia pastoris), Kluyveromyces marxianus, Cryptococcus albidus, Lipomyces lipofera, Lipomyces starkeyi, Rhodospondium toruloides, Rhodotorula glutinis and Trichosporon pullulan, expressing any of the enzymes described herein.

In particular, any of the above-mentioned modifications may be introduced in a Y. lipolytica cell expressing a TPS such as LdTPS8 (SEQ ID NO 1) and a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned. Preferably the CYP is any of NtEAH (SEQ ID NO 2) and/or DsEAH (SEQ ID NO 11). For example, the yeast cell may be a Y. lipolytica cell expressing a TPS such as LdTPS8 (SEQ ID NO 1), a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), a CPR and or a functional variant thereof having at least 65% identity, homology or similarity to any of the aforementioned

In other embodiments, any one or more of the above-mentioned modifications may be introduced in a S. cerevisiae cell expressing a TPS such as LdTPS8 (SEQ ID NO 1) and a CYP such as NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned. Preferably the Y. lipolytica and/or the S. cerevisiae cell also expresses a CPR, such as a CPR disclosed herein, for example in the sections “Cytochrome P450 reductase” and Production of hemandulcin. The CYP is preferably any of NtEAH (SEQ ID NO 2) and/or DsEAH (SEQ ID NO 11).

### Expression Systems and Nucleic Acids

Provided herein are expression systems useful for expression in a yeast cell being capable of producing (+)-epi-α-bisabolol, hemandulcin and/or one or more derivatives thereof as disclosed herein. Provided are also nucleic acids and/or yeast cell comprising said nucleic acids useful for producing (+)-epi-α-bisabolol, hemandulcin and/or one or more derivatives thereof. The present nucleic acids may be provided as one or more nucleic acid molecules, nucleic acid constructs or polynucleotides, for example they may be comprised in one or more vectors and/or expression systems. Such nucleic acids may be introduced in the cell by methods known in the art. The term expression system, nucleic acid constructs and polynucleotides may be used interchangeably herein. Said nucleic acid constructs, polynucleotides and/or expression systems may be useful for expression in, engineering and/or modifying a yeast cell.

It will be understood that throughout the present disclosure, the term “nucleic acid encoding a polypeptide” and/or “polynucleotide encoding a polypeptide” shall refer to a nucleic acid such as a nucleic acid molecule or nucleic acid construct capable of encoding a polypeptide, a protein or a fragment thereof. Such nucleic acid molecules be open reading frames or genes, or fragments thereof.

In one aspect, the present disclosure provides an expression system for expression in a yeast cell, comprising:
i. a nucleic acid encoding at least one (+)-epi-alpha-bisabolol synthase, preferably a heterologous (+)-epi-alpha-bisabolol synthase such as Lippia dulcis terpene synthase 8 (LdTPS8) (EC 4.2.3.138) as set forth in SEQ ID NO 1 or a functional variant thereof having at least 70% identity, homology or similarity thereto;
ii. a nucleic acid encoding at least one cytochrome P450 enzyme (EC), preferably a heterologous cytochrome P450 enzyme, for example a heterologous plant cytochrome P450 enzyme such as Nicotiana tabacum 5-epi-aristolocene dihydroxylase (NtEAH) as set forth in SEQ ID NO 2, DsEAH as set forth in SEQ ID NO 11), Solanum lycopersicum premnaspirodiene oxygenase-like protein (SIEAH) as set forth in SEQ ID NO 9, CcEAH as set forth in SEQ ID NO 13 and/or Capsicum annuum cytochrome P450 71D7-like protein (CaEAH) as set forth in SEQ ID NO 15, or a functional variant thereof having at least 65% identity, homology or similarity thereto; and
iii. optionally a nucleic acid encoding at least one cytochrome P450 reductase, preferably a heterologous cytochrome P450 reductase such as Arabidopsis thaliana cytochrome P450 reductase ATR2 (AtATR2) (EC 1.6.2.4) as set forth in SEQ ID NO 3 or a functional variant thereof having at least 70% identity, homology or similarity thereto.

In some embodiments, the expression system comprises a further nucleic acid encoding at least one further cytochrome P450 reductase (CPR), preferably a heterologous CPR such as Lippia dulcis CPR LdCPR1 as set forth in SEQ ID NO 7, or a functional variant thereof having at least 70% identity, homology or similarity thereto.

In some embodiments, the nucleic acid encoding at least one CPR and/or the nucleic acid encoding the at least one further CPR is a heterologous CPR, optionally a plant CPR. In some embodiments, said plant CPR is selected from a Lippia dulcis CPR such as LdCPR1 (SEQ ID NO 7), a Arabidopsis thaliana CPR such as AtATR2 (NP_194750.1) or AtATR1 (NP_194183.1), a Medicago truncatula CPR such as MtCPR1 (XP_003602898.1) or MtCPR2 (XP_003610109.1), a Lotus japonicas CPR such as LjCPR1 or LjCPR2 (BAG68945.1), or Glycyrrhiza uralensis CPR such as GuCPR1 (AUG98241.1) or GuCPR2 (QCZ35624.1), or a functional variant thereof having at least 70% identity, homology or similarity thereto, encoding a CPR.

Thus, in some embodiments, the present disclosure provides an expression system for expression in a yeast cell, said expression system comprising:
i. a nucleic acid encoding the at least one (+)-epi-alpha-bisabolol synthase, preferably said nucleic acid comprises or consists of LdTPS8 as set forth in SEQ ID NO 4 or a homologue thereof having at least 70% identity, homology or similarity thereto;
ii. a nucleic acid encoding the at least one cytochrome P450 enzyme, preferably said nucleic acid comprises or consists of:
   i. NtEAH as set forth in SEQ ID NO 5,
   ii. SIEAH as set forth in SEQ ID NO 10,
   iii. DsEAH as set forth in SEQ ID NO 12,
   iv. CcEAH as set forth in SEQ ID NO 14,
   v. CaEAH as set forth in SEQ ID NO 16,
      or a homologue thereof having at least 65% identity, homology or similarity thereto; and
iii. optionally a nucleic acid encoding the at least one cytochrome P450 reductase, preferably said nucleic acid comprises or consists of AtATR2 as set forth in SEQ ID NO 6 or a homologue thereof having at least 70% identity, homology or similarity thereto; and
iv. optionally a nucleic acid encoding the at least one further cytochrome P450 reductase, preferably said nucleic acid comprises or consists of LdCPR1 as set forth in SEQ ID NO 8 or a homologue thereof having at least 70% identity, homology or similarity thereto.

In some embodiments, the expression system comprises one or more nucleic acids comprising LdTPS8 as set forth in SEQ ID NO 4, NtEAH as set forth in SEQ ID NO 5, AtATR2 as set forth in SEQ ID NO 6, and/or LdCPR1 as set forth in SEQ ID NO 8 or homologues thereof having at least 65% identity, homology or similarity to SEQ ID NO 4, SEQ ID NO 5, SEQ ID NO 6, and/or SEQ ID NO 8, respectively, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity, or homology to SEQ ID NO 4, SEQ ID NO 5, SEQ ID NO 6, and/or SEQ ID NO 8, respectively.

In some embodiments, the expression system comprises one or more nucleic acids comprising LdTPS8 as set forth in SEQ ID NO 4 and NtEAH as set forth in SEQ ID NO 5 or homologues thereof having at least 65% identity, homology or similarity to SEQ ID NO 4 and SEQ ID NO 5, respectively. In other embodiments, the expression system comprises one or more nucleic acids comprising LdTPS8 as set forth in SEQ ID NO 4, NtEAH as set forth in SEQ ID NO 5 and AtATR2 as set forth in SEQ ID NO 6 or homologues thereof having at least 65% identity, homology or similarity to SEQ ID NO 4, SEQ ID NO 5 and SEQ ID NO 6, respectively. In some embodiments, the expression system comprises one or more nucleic acids comprising LdTPS8 as set forth in SEQ ID NO 4, NtEAH as set forth in SEQ ID NO 5, and LdCPR1 as set forth in SEQ ID NO 8 or homologues thereof having at least 65% identity, homology or similarity to SEQ ID NO 4, SEQ ID NO 5 and SEQ ID NO 8, respectively. In other embodiments, the expression system comprises one or more nucleic acids comprising LdTPS8 as set forth in SEQ ID NO 4, NtEAH as set forth in SEQ ID NO 5, AtATR2 as set forth in SEQ ID NO 6, and LdCPR1 as set forth in SEQ ID NO 8 or homologues thereof having at least 65% identity, homology or similarity to SEQ ID NO 4, SEQ ID NO 5, SEQ ID NO 6, and SEQ ID NO 8, respectively.

In some embodiments, the expression system comprises one or more nucleic acids comprising LdTPS8 (SEQ ID NO 4), and SIEAH (SEQ ID NO 10), DsEAH (SEQ ID NO 12), CcEAH (SEQ ID NO 14), or CaEAH (SEQ ID NO 16), or homologues thereof having at least 65% identity, homology or similarity to any of the aforementioned. In other embodiments, the expression system comprises one or more nucleic acids comprising LdTPS8 (SEQ ID NO 4), SIEAH (SEQ ID NO 10), DsEAH (SEQ ID NO 12), CcEAH (SEQ ID NO 14), or CaEAH (SEQ ID NO 16), and AtATR2 (SEQ ID NO 6), or homologues thereof having at least 65% identity, homology or similarity to any of the aforementioned. In some embodiments, the expression system comprises one or more nucleic acids comprising LdTPS8 (SEQ ID NO 4), SIEAH (SEQ ID NO 10), DsEAH (SEQ ID NO 12), CcEAH (SEQ ID NO 14), or CaEAH (SEQ ID NO 16), and LdCPR1 (SEQ ID NO 8), or homologues thereof having at least 65% identity, homology or similarity to any of the aforementioned.

In other embodiments, the expression system further comprises one or more promoters such as PrTEF, PrGPD, or homologues thereof having at least 70% identity, homology or similarity thereto, respectively.

In some embodiments, one or more of the at least one (+)-epi-alpha-bisabolol synthase nucleic acid, the at least one cytochrome P450 enzyme nucleic acid, the at least one cytochrome P450 reductase nucleic acid and/or the at least one further P450 reductase nucleic acid is codon-optimised.

Preferably, the yeast cell is as defined herein above in “Yeast cell” and/or anywhere else herein.

In some embodiments, said yeast cell comprising the expression system disclosed herein, is capable of producing hemandulcin and/or one or more derivatives thereof.

In some embodiments, each of the nucleic acids encoding each of the present polynucleotides, i.e. a (+)-epi-alpha-bisabolol synthase, a cytochrome P450 enzyme, and a cytochrome P450 reductase may be designed to be integrated within the genome of the yeast cell and/or they may be within one or more vectors comprised within the yeast cell and/or a combination of both integrated within the genome and within one or more vectors comprised within the yeast cell. In some embodiments, the nucleic acids encoding each of the present polynucleotides, i.e. a (+)-epi-alpha-bisabolol synthase, a cytochrome P450 enzyme, and a cytochrome P450 reductase may be designed to be integrated within the genome of the yeast cell. In some embodiments, the expression system comprising one or more of the present polynucleotides, such as the at least one (+)-epi-alpha-bisabolol synthase, the at least one cytochrome P450 enzyme, the at least one cytochrome P450 reductase, and the at least one further cytochrome P450 reductase may be designed to be integrated within the genome of the yeast cell.

In some embodiments, one or more of the nucleic acids encoding each of the present polynucleotides may be integrated in the genome of said yeast cell. Methods for integrating a nucleic acid are well known in the art. Thus, in some embodiments the polynucleotide of interest is encoded by introduction of a heterologous nucleic acid in the yeast cell. The nucleic acid such as heterologous nucleic acid encoding said polynucleotide may be codon-optimised, and/or may comprise features that can help improve the polynucleotide. Such modifications include, but are not limited to, introduction of localisation signals, gain-of-function and/or loss-of-function mutations, fusion of the polypeptide to a marker or a tag such as fluorescent tag, insertion of an inducible promoter, introduction of modifications conferring increased stability and/or half-life.

The introduction of the heterologous nucleic acid encoding the polynucleotide of interest can be performed by methods known in the art. The skilled person will recognise that such methods include, but are not limited to: cloning and homologous recombination-based methods. Cloning methods may involve the design and construction of a plasmid e.g. in an organism such as Escherichia coli. The plasmid may be an integrative or a non-integrative vector. Cloning-free methods comprise homologous recombination-based methods such as adaptamer-mediated PCR or gap repair. Such methods often result in integration of the heterologous nucleic acid in the genome of the yeast cell.

The nucleic acids encoding the polynucleotides of interest may be present in high copy number. The nucleic acids encoding the polynucleotides of interest may be present in low copy number.

In some embodiments, the nucleic acid, nucleic acid construct, polynucleotide and/or expression system further comprises or consists of one or more vectors, such as an integrative vector or a replicative vector. In some embodiments, the vector is a high copy replicative vector. In some embodiments, the vector is a low copy replicative vector. In some embodiments, the vector is an episomal plasmid.

Each of the nucleic acids comprised within the present expression system(s) may be present in multiple copies in the cell. In some embodiments, at least one of the nucleic acids is present in the cell in at least 2 copies, such as at least 3 copies, such as at least 4 copies, such as at least 5 copies, such as at least 10 copies, such as at least 20 copies, such as at least 30 copies, such as at least 50 copies, such as at least 70 copies, such as at least 80 copies, such as at least 100 copies, such as at least 200 copies, or more. In some embodiments, all of the nucleic acids are present in the cell at least 2 copies, such as at least 3 copies, such as at least 4 copies, such as at least 5 copies, such as at least 10 copies, such as at least 20 copies, such as at least 30 copies, such as at least 50 copies, such as at least 70 copies, such as at least 80 copies, such as at least 100 copies, such as at least 150 copies, such as at least 200 copies, or more.

The expression systems may, in addition to the at least one (+)-epi-alpha-bisabolol synthase nucleic acid, the at least one cytochrome P450 enzyme nucleic acid, the at least one cytochrome P450 reductase nucleic acid and/or the at least one further P450 reductase nucleic acid disclosed above, also comprise additional nucleic acids or polynucleotides useful for introducing additional modifications in the yeast cell, to obtain cells as disclosed in “Other modifications” above or anywhere herein. Designing such additional nucleic acids, nucleic acid constructs or polynucleotides can be performed as is known in the art.

The nucleic acid constructs may be one or more PCR products or one or more synthetic DNA molecules.

In a further aspect, the present disclosure provides a yeast cell comprising:
i. a nucleic acid encoding the at least one (+)-epi-alpha-bisabolol synthase, preferably wherein said nucleic acid comprises or consists of LdTPS8 as set forth in SEQ ID NO 4, or a homologue thereof having at least 70% identity, homology or similarity thereto;
ii. a nucleic acid encoding the at least one cytochrome P450 enzyme, preferably wherein said nucleic acid comprises or consists of:
   i. NtEAH as set forth in SEQ ID NO 5,
   ii. DsEAH as set forth in SEQ ID NO 12,
   iii. SIEAH as set forth in SEQ ID NO 10,
   iv. CcEAH as set forth in SEQ ID NO 14,
   v. CaEAH as set forth in SEQ ID NO 16,
      or homologues thereof having at least 65% identity, homology or similarity thereto; and
iii. a nucleic acid encoding the at least one plant cytochrome P450 reductase, preferably wherein said nucleic acid comprises or consists of AtATR2 as set forth in SEQ ID NO 6, or a homologue thereof having at least 70% identity, homology or similarity thereto; and
iv. optionally a nucleic acid encoding the at least one further cytochrome P450 reductase, preferably wherein said nucleic acid comprises or consists of LdCPR1 as set forth in SEQ ID NO 8, or a homologue thereof having at least 70% identity, homology or similarity thereto.

The at least one (+)-epi-alpha-bisabolol synthase nucleic acid, the at least one plant cytochrome P450 enzyme nucleic acid, the at least one cytochrome P450 reductase nucleic acid and/or the at least one further P450 reductase nucleic acid may be native or codon-optimised.

The yeast cell may be a yeast cell that is useful for producing (+)-epi-α-bisabolol, hemandulcin and/or one or more derivatives thereof, such as a yeast cell disclosed herein and in particular in the section “Yeast cell”.

### Methods for Producing Hemandulcin

The present disclosure relates to methods for producing hemandulcin and/or one or more derivatives thereof. The yeast cells, expression systems and nucleic acids disclosed herein are useful for microbial-based production of hemandulcin and/or one or more derivatives thereof.

Throughout the present disclosure, it will be understood that the yeast cells disclosed herein can produce the compounds of interest listed herein such as hemandulcin when incubated in a cultivation medium under conditions that enable the cells to grow and produce the desired compound and/or product. From the description of the yeast cells, also sometimes referred to as cells, production host cells, production hosts or host cells, provided herein, and knowing the type of host cell used, the skilled person will not have difficulties in identifying suitable cultivation media and/or conditions to achieve production of said compounds and/or products.

In particular, the cultivation may be performed aerobically or anaerobically, at temperatures and at pH suitable for supporting growth of the cells. The cultivation medium should include the required nutrients, and may be supplemented with precursors as applicable. The time of cultivation will vary depending on which cell is used, but can easily be adapted by the skilled person.

In one aspect, the present disclosure provides a method for producing hernandulcin and/or one or more derivatives thereof in a yeast cell, said method comprising the steps of:
i. providing a yeast cell disclosed herein; and
ii. incubating said yeast cell in a medium,
   whereby hemandulcin and/or one or more derivatives thereof is produced.

Also provided herein are yeast cells (+)-epi-α-bisabolol, hemandulcin and/or one or more derivatives thereof comprising an expression system disclosed herein above and anywhere else herein, in particular in the section “Expression systems”.

Indeed, yeast cells useful for producing (+)-epi-α-bisabolol, hemandulcin and/or one or more derivatives thereof are disclosed herein above and anywhere else herein, in particular in the section “Yeast cell”.

Also provided herein is (+)-epi-α-bisabolol, hemandulcin and/or one or more derivatives thereof obtainable by the methods disclosed herein.

### Recovery

The present methods may comprise a further step of recovering and/or purifying the compounds and/or products such as hemandulcin and/or one or more derivatives obtained by the methods and/or yeast cells disclosed herein. Methods for recovering and/or purifying (+)-epi-α-bisabolol, hemandulcin and/or one or more derivatives thereof obtained by the present disclosure are known in the art, for example ethyl acetate extraction and silica column purification for (+)-epi-α-bisabolol, and supercritical fluid extraction for hemandulcin as described by Attia et al., 2012 or De Oliveira et al., 2012.

For example, the step of recovering the compound(s) such as hemandulcin and/or one or more derivatives may comprise separating the cell culture in a solid phase to obtain a cell phase and in a liquid phase to obtain a supernatant. The compounds may be present in the supernatant and/or the cell phase. The cell phase is also known as cell pellet. Supercritical fluid extraction may be used to recover one or more compounds of interest from the cell phase such as described by De Oliveira et al., 2012.

The present methods may further comprise a step of converting hemandulcin and/or one or more derivatives to one or more further derivatives thereof.

Thus, in one aspect the present methods may further comprise the steps of:
i. recovering the hemandulcin and/or the one or more derivatives thereof; and
ii. optionally converting said hemandulcin and/or the one or more derivatives thereof to one or more further derivatives thereof; and/or
iii. formulating said hemandulcin and/or the one or more derivatives thereof in a composition.

The composition may be a food composition and/or a beverage.

Step ii of the method above may be performed prior to or after step i. Thus, step i may be performed prior to or after step ii.

Yeast cells useful for producing (+)-epi-α-bisabolol, hemandulcin and/or one or more derivatives thereof are disclosed herein above and anywhere else herein, in particular in the section “Yeast cell”.

Also provided herein is (+)-epi-α-bisabolol, hemandulcin and/or one or more derivatives thereof obtainable by the methods disclosed herein.

In some embodiments, the method is for production of hernandulcin and/or one or more derivatives thereof and further comprises a step of recovering said hernandulcin and/or one or more derivatives thereof. In some embodiments, the method is for production of hemandulcin and further comprises a step of recovering said hemandulcin.

In some embodiments, the method is for production of hemandulcin and/or one or more derivatives thereof and further comprises a step of converting said hernandulcin and/or one or more derivatives thereof to one or more further derivatives thereof.

Conversion of hemandulcin and/or one or more derivatives into one or more further derivatives thereof may be performed by chemical synthesis, such as by contacting said hemandulcin and/or one or more derivative with one or more chemical reagents to react the compounds and reagents, whereby hemandulcin and/or one or more derivatives are being converted into to one or more further derivatives thereof said one or more further derivatives are produced.

Thus, in some embodiments the method is for production of hemandulcin and/or one or more derivatives thereof and further comprises a step of converting said hemandulcin and/or one or more derivatives thereof to a derivative thereof. In some embodiments the method is for production of hemandulcin and/or one or more derivatives thereof and further comprises a step of converting said hemandulcin and/or one or more derivatives thereof to one or more further derivatives thereof. Further, in some embodiments the method is for production of hemandulcin and further comprises a step of converting said hemandulcin to one or more derivatives thereof. In some embodiments the method is for production of hemandulcin and further comprises a step of converting said hemandulcin to one or more derivatives thereof and further to one or more further derivatives thereof.

In some embodiments, the method is for production of hemandulcin and further comprise a step of recovering said hemandulcin as well as converting said hemandulcin into a derivative. In some embodiments, the method is for production of hemandulcin and further comprises a step of recovering said hemandulcin as well as converting said hemandulcin into one or more further derivatives thereof.

A derivative of hemandulcin is for example 4-hydroxy-hemandulcin. Thus, provided herein are yeast cells, methods and/or use of enzymes useful for production of 4-hydroxy-hemandulcin.

### Products and Compositions

Also provided herein is (+)-epi-α-bisabolol, hemandulcin and/or one or more derivatives thereof obtainable by the methods disclosed herein. Thus, disclosed herein is hemandulcin obtainable by the methods disclosed herein.

Provided herein is also a fermentation liquid comprising hemandulcin and/or one or more derivatives thereof obtained by the methods disclosed herein, such as in the section “Methods for producing hemandulcin”. Said fermentation liquid may be processed further to obtain a processed fermentation liquid. In some embodiments, the fermentation liquid comprises at least 50% yeast cell debris. In other words, the fermentation liquid may comprise 50% yeast cell debris. In some embodiments, at least 50% of cellular material, such as yeast cell debris, is separated from the fermentation liquid. Thus, in other embodiments, the fermentation liquid comprises less than 50% yeast cell debris and/or cellular material.

Provided herein is also a composition comprising hemandulcin and/or one or more derivatives thereof obtained by the methods disclosed herein. Said composition may be a food composition and/or a beverage. Thus, provided herein is also a food composition and/or a beverage comprising hemandulcin and/or one or more derivatives thereof, preferably wherein said hemandulcin and/or one or more derivatives thereof is obtained by a method and/or a yeast cell disclosed herein.

### Titer

The yeast cells and/or methods disclosed in the present disclosure are capable of producing (+)-epi-α-bisabolol, hemandulcin and/or one or more derivatives thereof. In preferred embodiments, the yeast cells are capable of producing hemandulcin.

A method for quantifying hemandulcin is provided in the section “Example 1—Materials and Methods”, particularly in “Cultivation and sample preparation”.

In some embodiments, the yeast cells disclosed herein are capable of producing (+)-epi-α-bisabolol, hemandulcin and/or one or more derivatives thereof with a titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

In some embodiments, the yeast cells disclosed herein are capable of producing hemandulcin and/or one or more derivatives thereof with a titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

In some embodiments, the yeast cells disclosed herein are capable of producing hemandulcin with a titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

The methods disclosed herein are capable of yielding a titer of (+)-epi-α-bisabolol, hemandulcin and/or one or more derivatives thereof of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

In some embodiments, the methods disclosed herein are capable of yielding a titer of hemandulcin and/or one or more derivatives thereof of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

In some embodiments, the methods disclosed herein are capable of yielding a titer of hemandulcin of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

Thus, the yeast cell producing hemandulcin with any of the above titers may be a yeast cell belonging to a genus selected from Saccharomyces such as Saccharomyces cerevisiae and Saccharomyces boulardi, Pichia such as Komagetella phaffi (Pichia pastoris), Yarrowia such as Y. lipolytica, Kluyveromyces such as Kluyveromyces marxianus, Candida, Rhodotorula such as Rhodotorula glutinis, Rhodosporidium such as Rhodosporidium toruloides, Cryptococcus such as Cryptococcus albidus, Trichosporon such as Trichosporon pullulan and Lipomyces such as Lipomyces lipofera or Lipomyces starkeyi.

In preferred embodiments, the yeast cell producing hemandulcin is a Y. lipolytica cell capable of producing hemandulcin and/or one of more derivatives thereof with a titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more. Said Y. lipolytica cell may be used in a method of producing hemandulcin and/or one of more derivatives thereof yielding a titer of hemandulcin of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

In other preferred embodiments, the yeast cell producing hernandulcin is a S. cerevisiae cell capable of producing hemandulcin and/or one of more derivatives thereof with a titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more. Said S. cerevisiae cell may be used in a method of producing hemandulcin and/or one of more derivatives thereof yielding a titer of hemandulcin of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

### Uses

Further disclosed herein is the use of a Nicotana cytochrome P450 enzyme, preferably a Nicotana tabacum cytochrome P450 enzyme, in a method for producing hemandulcin and/or one or more derivatives, optionally wherein the Nicotana tabacum cytochrome P450 enzyme is the polypeptide as set forth in SEQ ID NO 2, or a functional variant thereof having at least 65% identity, homology or similarity thereto. Disclosed herein is also the use of a Datura cytochrome P450 enzyme, preferably a Datura stramonium cytochrome P450 enzyme, in a method for producing hemandulcin and/or one or more derivatives, optionally wherein the Datura stramonium cytochrome P450 enzyme is DsEAH as set forth in SEQ ID NO 11, or a functional variant thereof having at least 65% identity, homology or similarity thereto. Also disclosed is the use of a Solanum cytochrome P450 enzyme, preferably a Solanum lycopersicum cytochrome P450 enzyme, in a method for producing hemandulcin and/or one or more derivatives, optionally wherein the Solanum lycopersicum cytochrome P450 enzyme is SIEAH as set forth in SEQ ID NO 9, or a functional variant thereof having at least 65% identity, homology or similarity thereto. Further disclosed herein is use of a Capsicum cytochrome P450 enzyme, preferably a Capsicum chinense cytochrome P450 enzyme and/or a Capsicum annuum cytochrome P450 enzyme, in a method for producing hemandulcin and/or one or more derivatives, optionally wherein the Capsicum chinense cytochrome P450 enzyme is CcEAH as set forth in SEQ ID NO 13, the Capsicum annuum cytochrome P450 enzyme is CaEAH as set forth in SEQ ID NO 15, or a functional variants thereof having at least 65% identity, homology or similarity to any of the aforementioned.

In some embodiments, the polypeptide comprises the sequence as set forth in SEQ ID NO 2, SEQ ID NO 9, SEQ ID NO 11, SEQ ID NO 13 and/or SEQ ID NO 15, with the exception that at the most 54 residues are mutated, such as at the most 53 residues, such as at the most 52 residues, for example 51 residues are mutated, such as at the most 50 residues, for example at the most 49 residues, such as at the most 48 residues, for example at the most 45 residues, such as at the most 40 residues, for example at the most 35 residues, such as at the most 30 residues, for example at the most 25 residues, such as at the most 20 residues, for example at the most 15 residues, such as at the most 10 residues, for example at the most 5 residues, or less. Thus, in some embodiments. Thus, in some embodiments, the polypeptide comprises the sequence as set forth in SEQ ID NO 2, SEQ ID NO 9, SEQ ID NO 11, SEQ ID NO 13 and/or SEQ ID NO 15, with the exception that at the most 54 residues are mutated, for example between 54 and 51 residues are mutated, such as between 51 and 45 residues, such as between 35 and 45 residues, such as between 25 and 35 residues, such as between 15 and 25 residues, such as between 5 and 15 residues, or less.

In some embodiments, the use comprises expressing the Nicotana cytochrome P450 enzyme, preferably a Nicotana tabacum cytochrome P450 enzyme, optionally wherein the Nicotiana tabacum cytochrome P450 enzyme is NtEAH as set forth in SEQ ID NO 2, or functional variants thereof having at least 65% identity, homology or similarity thereto in a yeast cell, wherein said yeast cell is as disclosed in “Yeast cell” herein above, or anywhere else herein.

In further some embodiments, the use comprises expressing the Datura cytochrome P450 enzyme, preferably a Datura stramonium cytochrome P450 enzyme, optionally wherein the Datura stramonium cytochrome P450 enzyme is DsEAH as set forth in SEQ ID NO 11, or functional variants thereof having at least 65% identity, homology or similarity thereto in a yeast cell, wherein said yeast cell is as disclosed in “Yeast cell” herein above, or anywhere else herein.

In other embodiments, the use comprises expressing the Solanum cytochrome P450 enzyme in a yeast cell, preferably a Solanum lycopersicum cytochrome P450 enzyme, optionally wherein the Solanum lycopersicum cytochrome P450 enzyme is SIEAH as set forth in SEQ ID NO 9, or functional variants thereof having at least 65% identity, homology or similarity thereto in a yeast cell, wherein said yeast cell is as disclosed in “Yeast cell” herein above, or anywhere else herein.

In further embodiments, the use comprises expressing the Capsicum cytochrome P450 enzyme in a yeast cell, preferably a Capsicum chinense cytochrome P450 enzyme and/or a Capsicum annuum cytochrome P450 enzyme, optionally wherein the Capsicum chinense cytochrome P450 enzyme is CcEAH as set forth in SEQ ID NO 13, the Capsicum annuum cytochrome P450 enzyme is CaEAH as set forth in SEQ ID NO 15, or functional variants thereof having at least 65% identity, homology or similarity to any of the aforementioned in a yeast cell, wherein said yeast cell is as disclosed in “Yeast cell” herein above, or anywhere else herein.

In some embodiments, the use of the Nicotiana cytochrome P450 enzyme, preferably a Nicotiana tabacum cytochrome P450 enzyme such as NtEAH as set forth in SEQ ID NO 2, or functional variants thereof having at least 65% identity, homology or similarity thereto, is in the method for producing hemandulcin and/or one or more derivatives as disclosed in “Methods for producing hemandulcin” herein above, or anywhere else herein. In other embodiments, the use of the Datura cytochrome P450 enzyme, preferably a Datura stramonium cytochrome P450 enzyme, optionally wherein the Datura stramonium cytochrome P450 enzyme is DsEAH as set forth in SEQ ID NO 11, or functional variants thereof having at least 65% identity, homology or similarity thereto, is in the method for producing hemandulcin and/or one or more derivatives as disclosed in “Methods for producing hemandulcin” herein above, or anywhere else herein. In further other embodiments, the use of the Solanum cytochrome P450 enzyme in a yeast cell, preferably a Solanum lycopersicum cytochrome P450 enzyme, optionally wherein the Solanum lycopersicum cytochrome P450 enzyme is SIEAH as set forth in SEQ ID NO 9, or functional variants thereof having at least 65% identity, homology or similarity thereto, is in the method for producing hemandulcin and/or one or more derivatives as disclosed in “Methods for producing hemandulcin” herein above, or anywhere else herein. In further embodiments, the use of the Capsicum cytochrome P450 enzyme in a yeast cell, preferably a Capsicum chinense cytochrome P450 enzyme and/or a Capsicum annuum cytochrome P450 enzyme, optionally wherein the Capsicum chinense cytochrome P450 enzyme is CcEAH as set forth in SEQ ID NO 13, the Capsicum annuum cytochrome P450 enzyme is CaEAH as set forth in SEQ ID NO 15, or functional variants thereof having at least 65% identity, homology or similarity to any of the aforementioned, is in the method for producing hemandulcin and/or one or more derivatives as disclosed in “Methods for producing hemandulcin” herein above, or anywhere else herein.

Also provided herein is the use of nucleic acids, expression systems and/or yeast cells disclosed anywhere herein such as in “Expression systems” or “Yeast cell” herein above, for the production of hemandulcin and/or one or more derivatives thereof.

### EXAMPLES

### Example 1—Materials and Methods

### Strains and Media

A strain previously engineered for high mevalonate (MVA) pathway flux (ST9149) or without engineering (ST6512) were used to construct the hemandulcin producing strains (Arnesen et al., 2020; Marella et al., 2020) (Table 3). Both strains were based on the W29-strain (Y-63746), received as a kind gift from the ARS Culture Collection, NCAUR, USA.

The DH5α Escherichia coli strain was used for plasmids construction. Lysogeny broth (LB) media with 100 mg/L ampicillin was used to cultivate E. coli cells at 300 rpm shaking and 37° C. The Y. lipolytica cells were cultivated at 30° C. on media containing 10 g/L yeast extract, 20 g/L peptone, and 20 or 80 g/L glucose (YPD or YPD80) with 20 g/L agar added for solid media. Hygromycin (400 mg/L) or nourseothricin (250 mg/L) was added to the media for yeast cell selection.

### Strain Construction

Biobricks, plasmids and strains are listed in Table 1, 2 and 3, respectively Phusion U polymerase (Thermo Scientific) was used to PCR-amplify the biobricks, which were assembled into EasyCloneYALI plasmids by USER cloning (Holkenbrink et al., 2018). The USER reactions were transformed into E. coli and correct assembly was verified by sequencing.

The genes encoding the Lippia dulcis terpene synthase 8 (LdTPS8), L. dulcis cytochrome P450 reductase (LdCPR1), Nicodana tabacum 5-Epi-aristolochene dihydroxylase (EAH), and EAH-related enzymes from Solanum lycopersicum (SIEAH), Datura stramonium (DtEAH), S. commersonii (ScEAH, NCBI accession number for polypeptide: KAG5574796.1), Capsicum chinense (CcEAH), C. annuum (CaEAH), C. baccatum (CbEAH, NCBI accession number for polypeptide: PHT58219.1), and Hyoscyamus muticus (HmHPO, Uniprot accession number for polypeptide: A6YIH8 (C7D55_HYOMU)) were ordered as GeneArt String DNA fragments or full gene synthesis from Thermo Fischer Scientific. The Arabidopsis thaliana cytochrome P450 reductase 2 (AtATR2) gene was described in a previous publication (Kildegaard et al., 2021). The DNA-sequences were codon-optimized for Y. lipolytica.

The integration vectors were NotI-digested before lithium acetate transformation based on a previously described protocol (Holkenbrink et al., 2018). The genomic integration of the plasmids were confirmed by colony PCR with primers complementary to the genomic region and plasmid (Holkenbrink et al., 2018).

TABLE 1
Biobrick table

| Biobrick number | Template | Reference |
|---|---|---|
| BB3865 (<-PrGPD_PrTEFint->) | See reference (therein BB3865 | (Arnesen et |
| | (<-PrGDP_Tefint->) | al., 2020) |
| BB4645 (PrGPD_link_LdCPR1) | LdCPR1 synthetic gene | — |
| BB4646 (PrTEFint_link_LdTPS8) | LdTPS8 synthetic gene | — |
| BB5274 | pCfB6677 (pIntE_1-TPex20- | — |
| (pIntE_1_backbone_user) | TLip2) | |
| BB5282 (AtATR2<-PrGPD- | pCfB9247 (pIntF_3-AtATR2<- | — |
| PrTEFint_new) | PrGPD-PrTEFInt- | |
| | >MtCYP716A12) | |
| BB5284 (NtEAH) | NtEAH synthetic gene | — |
| BB5277 | pCfB10567 (pIntE_3_PrTEFint- | — |
| (IntE_3_backbone_PrTEFint_user) | >MdOSC1_mut-YISQE) | |
| BB6038 (LdTPS8_User) | pCfB10064 (pIntE4_LdCPR1<- | — |
| | PrGPD_PrTEFint->LdTPS8) | |
| BB5265 (PrTEFint_pcrUSER) | pCfB10567 (pIntE_3_PrTEFint- | — |
| | >MdOSC1_mut-YISQE) | |
| BB5275 (IntE_3_backbone_user) | pCfB6681 (pIntE_3-TPex20- | — |
| | TLip2) | |
| BB6096 | pCfB10924 (pIntE_1_pTEFint- | — |
| (IntE_1_backbone_PrTEFint_user) | >BM3) | |
| BB6077 (SIEAH) | SIEAH synthetic gene | — |
| BB6078 (DtEAH) | DtEAH synthetic gene | — |
| BB6079 (ScEAH) | ScEAH synthetic gene | — |
| BB6080 (CcEAH) | CcEAH synthetic gene | — |
| BB6081 (CaEAH) | CaEAH synthetic gene | — |
| BB6082 (CbEAH) | CbEAH synthetic gene | — |
| BB6083 (HmHPO) | pCfB10182 (intF3_PrTEFint- | — |
| | >HmHPO) | |

TABLE 2
Plasmid table

| | Parental | | |
|---|---|---|---|
| Plasmid number | plasmid | Biobricks | Reference |
| pCfB10064 | pCfB6679 | BB4645 | — |
| (pIntE4_LdCPR1<- | (pIntE_4- | (PrGPD_link_LdCPR1): BB3865 | |
| PrGPD_PrTEFint- | TPex20- | (<-PrGPD_PrTEFint->): BB4646 | |
| >LdTPS8) | TLip2) | (PrTEFint_link_LdTPS8) | |
| pCfB6638 (pNat- | see | see reference | (Holken |
| YLgRNA2_IntE_4) | reference | | brink et |
| | | | al., |
| | | | 2018) |
| pCfB8858 (pHphM- | — | — | Laboratory |
| YLgRNA2_IntE_1) | | | collection |
| pCfB10742 (pIntE_1- | — | BB5274 | — |
| AtATR2<-PrGPD- | | (pIntE_1_backbone_user): BB5282 | |
| PrTEFint->NtEAH) | | (AtATR2<-PrGPD- | |
| | | PrTEFint_new): BB5284 (NtEAH) | |
| pCfB6679 (pIntE_4- | see | see reference | (Holken |
| TPex20-TLip2) | reference | | brink et |
| | | | al., |
| | | | 2018) |
| pCfB6677 (pIntE_1- | see | see reference | (Holken |
| TPex20-TLip2) | reference | | brink et |
| | | | al., |
| | | | 2018) |
| pCfB9247 (pIntF_3- | see | see reference (therein pCfB9247 | (Arnesen |
| AtATR2<-PrGPD- | reference | (IntF_3-AtATR2<-GPD-tefInt- | et al., |
| PrTEFInt- | | >MtCYP716A12) | 2020) |
| >MtCYP716A12) | | | |
| pCfB8860 (pHphM- | — | — | Laboratory |
| YLgRNA2_IntE_3) | | | collection |
| pCfB6633 (pNat- | — | See reference | (Holken |
| YLgRNA2_IntE_1) | | | brink et |
| | | | al., |
| | | | 2018) |
| pCfB6637 (pNat- | — | See reference | (Holken |
| YLgRNA3_IntE_3) | | | brink et |
| | | | al., |
| | | | 2018) |
| pCfB11702 (pIntE_3- | — | BB5277 | — |
| PrTEFint>LdTPS8) | | (IntE_3_backbone_PrTEFint_{—} | |
| | | user): BB6038 (LdTPS8_User) | |
| pCfB11703 (pIntE_1- | — | BB5274 | — |
| PrTEFint->NtEAH) | | (IntE_1_backbone_user): BB5265 | |
| | | (PrTEFint_pcrUSER): BB5284 | |
| | | (NtEAH) | |
| pCfB11765 (pIntE3- | — | BB5275 | — |
| AtATR2-PrGPD- | | (IntE_3_backbone_user): BB5282 | |
| PrTEFint->LdTPS8) | | (AtATR2<-PrGPD- | |
| | | PrTEFint_new): BB6038 | |
| | | (LdTPS8_User) | |
| pCfB11766 (pIntE_1- | — | BB6096 | — |
| PrTEFint->SIEAH) | | (IntE_1_backbone_PrTEFint_{—} | |
| | | user): BB6077 (SIEAH) | |
| pCfB11767 (pIntE_1- | — | BB6096 | — |
| PrTEFint->DtEAH) | | (IntE_1_backbone_PrTEFint_{—} | |
| | | user): BB6078 (DtEAH) | |
| pCfB11768 (pIntE_1- | — | BB6096 | — |
| PrTEFint->ScEAH) | | (IntE_1_backbone_PrTEFint_{—} | |
| | | user): BB6079 (ScEAH) | |
| pCfB11769 (pIntE_1- | — | BB6096 | — |
| PrTEFint->CcEAH) | | (IntE_1_backbone_PrTEFint_{—} | |
| | | user): BB6080 (CcEAH) | |
| pCfB11770 (pIntE_1- | — | BB6096 | — |
| PrTEFint->CaEAH) | | (IntE_1_backbone_PrTEFint_{—} | |
| | | user): BB6081 (CaEAH) | |
| pCfB11771 (pIntE_1- | — | BB6096 | — |
| PrTEFint->CbEAH) | | (IntE_1_backbone_PrTEFint_{—} | |
| | | user): BB6082 (CbEAH) | |
| pCfB11772 (pIntE_1- | — | BB6096 | — |
| PrTEFint->HmHPO) | | (IntE_1_backbone_PrTEFint_{—} | |
| | | user): BB6083 (HmHPO) | |
| pCfB10924 | — | — | Laboratory |
| (pIntE_1_pTEFint- | | | collection |
| >BM3) | | | |
| pCfB10567 | | | (Arnesen |
| (pIntE_3_PrTEFint- | — | — | et al., |
| >MdOSC1_mut- | | | 2022) |
| YISQE) | | | |
| pCfB10182 | — | — | Laboratory |
| (intF3_PrTEFint- | | | collection |
| >HmHPO) | | | |

TABLE 3
Strain table. All strains are Yarrowia lipolytica.

| | | Parental | | |
|---|---|---|---|---|
| Strain | Genotype | strain | Vector elements | Reference |
| ST10274 | ST9149 | ST9149 | pCfB6638 (pNat- | — |
| | IntE4_LdCPR1<- | | YLgRNA2_IntE_4): | |
| | PrGPD_PrTEFint- | | pCfB10064 | |
| | >LdTPS8 | | (pIntE4_LdCPR1<- | |
| | | | PrGPD_PrTEFint- | |
| | | | >LdTPS8) | |
| ST10275 | ST6512 | ST6512 | pCfB6638 (pNat- | — |
| | IntE4_LdCPR1<- | | YLgRNA2_IntE_4): | |
| | PrGPD_PrTEFint- | | pCfB10064 | |
| | >LdTPS8 | | (pIntE4_LdCPR1<- | |
| | | | PrGPD_PrTEFint- | |
| | | | >LdTPS8) | |
| ST11474 | ST10274 IntE_1- | ST10274 | pCfB8858 (pHphM- | — |
| (HRN1- | AtATR2<-PrGPD- | | YLgRNA2_IntE_1): | |
| producer) | PrTEFint->NtEAH | | pCfB10742 (pIntE_1- | |
| | | | AtATR2<-PrGPD- | |
| | | | PrTEFint->NtEAH) | |
| ST11669 | ST10275 IntE_1- | ST10275 | pCfB8858 (pHphM- | — |
| (HRN2- | AtATR2<-PrGPD- | | YLgRNA2_IntE_1): | |
| producer) | PrTEFint->NtEAH | | pCfB10742 (pIntE_1- | |
| | | | AtATR2<-PrGPD- | |
| | | | PrTEFint->NtEAH) | |
| ST6512 | MATa | See | See reference | (Marella et |
| | ku70Δ::PrTEF1- | reference | | al., 2020) |
| | Cas9- | | | |
| | TTef12::PrGPD- | | | |
| | DsdA-TLip2 | | | |
| ST9149 | MATa | See | See reference | (Arnesen et |
| | ku70Δ::PrTEF1- | reference | | al., 2020) |
| | Cas9- | | | |
| | TTef12::PrGPD- | | | |
| | DsdA-TLip2 | | | |
| | IntC_2-HMG1<- | | | |
| | PrGPD-PrTEFint- | | | |
| | >ERG12 IntC_3- | | | |
| | SeACS<-PrGPD- | | | |
| | PrTEFint- | | | |
| | >YIACL1 IntD_1- | | | |
| | IDI1<-PrGPD- | | | |
| | PrTEFint- | | | |
| | >ERG20 | | | |
| | PERG11::pSQS1 | | | |
| ST12430 | ST6512 IntE3- | ST6512 | pCfB8860 (pHphM- | — |
| | PrTEFint- | | YLgRNA2_IntE_3): | |
| | >LdTPS8 | | pCfB11702 (pIntE_3- | |
| | | | PrTEFint->LdTPS8) | |
| ST12433 | ST12430 IntE_1- | ST12430 | pCfB6633 (pNat- | — |
| | PrTEFint->NtEAH | | YLgRNA2_IntE_1): | |
| | | | pCfB11703 (pIntE_1- | |
| | | | PrTEFint->NtEAH) | |
| ST12439 | ST12430 | ST12430 | pCfB6633 (pNat- | — |
| | pintE_1- | | YLgRNA2_IntE_1): | |
| | AtATR2<-PrGPD- | | pCfB10742 (pIntE_1- | |
| | PrTEFint->NtEAH | | AtATR2<-PrGPD- | |
| | | | PrTEFint->NtEAH) | |
| ST12475 | ST9149 IntE3- | ST9149 | pCfB6637 (pNat- | — |
| | AtATR2-PrGPD- | | YLgRNA3_IntE_3): | |
| | PrTEFint- | | pCfB11765 (pIntE3-AtATR2- | |
| | >LdTPS8 | | PrGPD-PrTEFint- | |
| | | | >LdTPS8) | |
| ST12476 | ST12475 IntE_1- | ST12475 | pCfB8858 (pHphM- | — |
| | PrTEFint->NtEAH | | YLgRNA2_IntE_1): | |
| | | | pCfB11703 (pIntE_1- | |
| | | | PrTEFint->NtEAH) | |
| ST12477 | ST12475 IntE_1- | ST12475 | pCfB8858 (pHphM- | — |
| | PrTEFint->SIEAH | | YLgRNA2_IntE_1): | |
| | | | pCfB11766 (pIntE_1- | |
| | | | PrTEFint->SIEAH) | |
| ST12478 | ST12475 IntE_1- | ST12475 | pCfB8858 (pHphM- | — |
| | PrTEFint->DtEAH | | YLgRNA2_IntE_1): | |
| | | | pCfB11767 (pIntE_1- | |
| | | | PrTEFint->DtEAH) | |
| ST12479 | ST12475 IntE_1- | ST12475 | pCfB8858 (pHphM- | — |
| | PrTEFint- | | YLgRNA2_IntE_1): | |
| | >ScEAH | | pCfB11768 (pIntE_1- | |
| | | | PrTEFint->ScEAH) | |
| ST12480 | ST12475 IntE_1- | ST12475 | pCfB8858 (pHphM- | — |
| | PrTEFint- | | YLgRNA2_IntE_1): | |
| | >CcEAH | | pCfB11769 (pIntE_1- | |
| | | | PrTEFint->CcEAH) | |
| ST12481 | ST12475 IntE_1- | ST12475 | pCfB8858 (pHphM- | — |
| | PrTEFint- | | YLgRNA2_IntE_1): | |
| | >CaEAH | | pCfB11770 (pIntE_1- | |
| | | | PrTEFint->CaEAH) | |
| ST12482 | ST12475 IntE_1- | ST12475 | pCfB8858 (pHphM- | — |
| | PrTEFint- | | YLgRNA2_IntE_1): | |
| | >CbEAH | | pCfB11771 (pIntE_1- | |
| | | | PrTEFint->CbEAH) | |
| ST12483 | ST12475 IntE_1- | ST12475 | pCfB8858 (pHphM- | — |
| | PrTEFint- | | YLgRNA2_IntE_1): | |
| | >HmHPO | | pCfB11772 (pIntE_1- | |
| | | | PrTEFint->HmHPO) | |

### Cultivation and Sample Preparation

For relative and non-quantitative analysis of hemandulcin, 2.5 mL of YPD80 in 24-well plates with an air-penetrable lid (EnzyScreen, NL) was inoculated with single yeast clones from glycerol stocks or plates, and cultivated for 72 hours at 30° C. and 300 rpm agitation. For quantitative hemandulcin analysis, precultures of 2.5 ml of YPD or YPD80 in 24-well plates were inoculated with single yeast clones from glycerol stock or plates and grown for 16-24 h at 30° C. with shaking. The optical densities at 600 nm (OD600) of the precultures were then measured with a VWR NanoPhotometer™ 7122 and the precultures were used to inoculate 2.5 mL of YPD80 in 24-deep well plates were inoculated to a starting OD of 0.1, which were grown at 30° C. with 300 rpm agitation for 72 hours. Alternatively, 10 μL of preculture was used to inoculate 2.5 mL of YPD80 in 24-deep well plates, which were grown at 30° C. with 225 rpm agitation for 72 hours. The cultures for quantitative analysis were performed with three replicates for each strain.

Cultivation broth was collected and centrifuged, whereafter the supernatant fraction was sampled for analysis. For cell extract analysis, 1 mL of cultivation broth was transferred to a 2 ml microtube (Sarstedt), which was centrifuged, and the supernatant discarded. The cell pellets were washed twice with water, by addition of 1 mL water, centrifugation, and water removal. Then 500 μL of 0.212-0.3 mm acid-washed glass beads and 1 mL ethanol (99%) were added to the cell pellets. Thereafter, the cells were disrupted with a Precellys®24 homogenizer (Bertin Corp.) using three cycles at 5000 rpm for 30 seconds each. The samples were then centrifuged and the ethanolic phase was sampled for analysis.

### Analytics

The LC-MS(MS) analysis was performed using a Dionex 3000 HPLC system connected to an Orbitrap Fusion Mass Spectrometer (Thermo Fisher Scientific, San Jose, CA). The chromatographic separation was achieved using a Waters ACQUITY BEH C18 (10 cm×2.1 mm, 1.7 μm) column equipped with an ACQUITY BEH C18 guard column kept at 40° C. The mobile phases consisted of MilliQ© water+0.1% formic acid (A) and acetonitrile+0.1% formic acid (B). The initial composition was 2% B, held for 0.8 min, followed by a linear gradient till 5% in 3.3 min, and to 100% B in 10 min and held for 1 min before going back to initial conditions. Re-equilibration time was 2.7 min. Flow rate was kept constant at 0.35 mL/min and injection volume was 1 uL. The MS(MS) measurement was done in positive-heated electrospray ionization (HESI) mode with a voltage of 2500 V acquiring in full MS/MS spectra (Data Dependent Acquisition-driven MS/MS) in the mass range of 70-1000 Da. The resolution was set at 120,000 for MS and to 30,000 for the MS2. Precursor ions were fragmented by High Energy Collision Dissociation (HCD) using collision energies of 20, 40, and 55.

### Example 2—Results

We attempted to find an enzyme capable of catalyzing the conversion of (+)-epi-α-bisabolol to hemandulcin. We searched the literature for potential plant cytochrome P450 candidates capable of oxygenating (+)-epi-α-bisabolol and tested the Nicodana tabacum 5-Epi-aristolochene dihydroxylase (NtEAH). NtEAH has been shown to oxygenate the allylic position on cyclic sesquiterpenes, thus converting premnaspirodiene into solavetivol and solavetivone, and valencene into nootkatol and nootkatone as examples (Takahashi et al., 2007).

A Y. lipolytica platform strain (ST9149, Arnesen et al., 2020) was previously engineered for sesquiterpenoid production was further engineered for hemandulcin production. We reconstituted a biosynthetic pathway towards hemandulcin by expressing the genes encoding the Lippia dulcis terpene synthase 8 (LdTPS8), L. dulcis cytochrome P450 reductase (LdCPR1), Arabidopsis thaliana cytochrome P450 reductase 2 (AtATR2), and NtEAH in ST9149 generating the HRN1-producer (Table 3, FIG. 1), LdTPS8, LdCPR1, AtATR2 and NtEAH, respectively. The genes were codon-optimized for expression in Y. lipolytica.

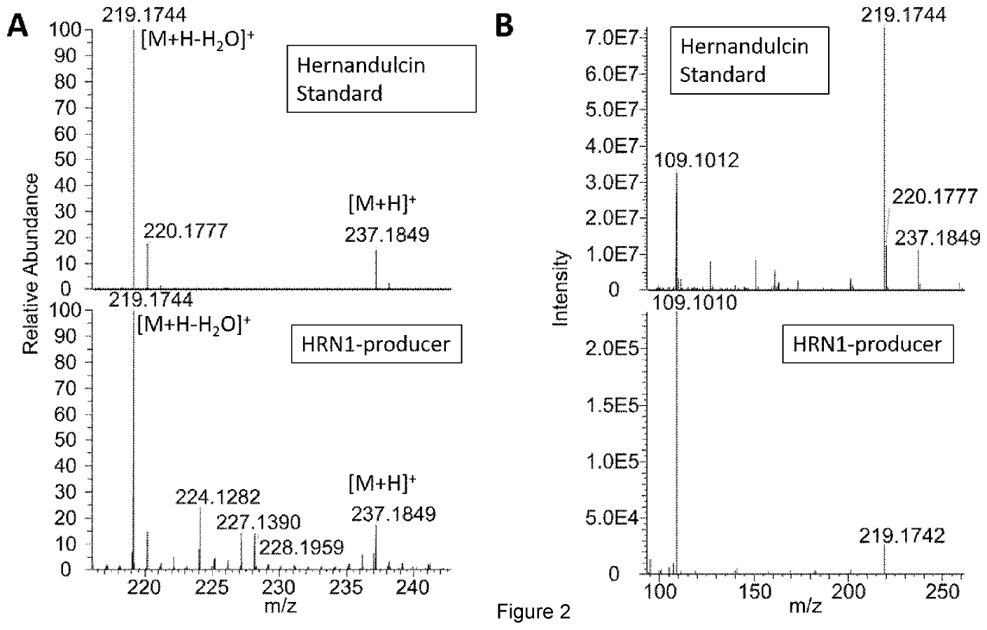

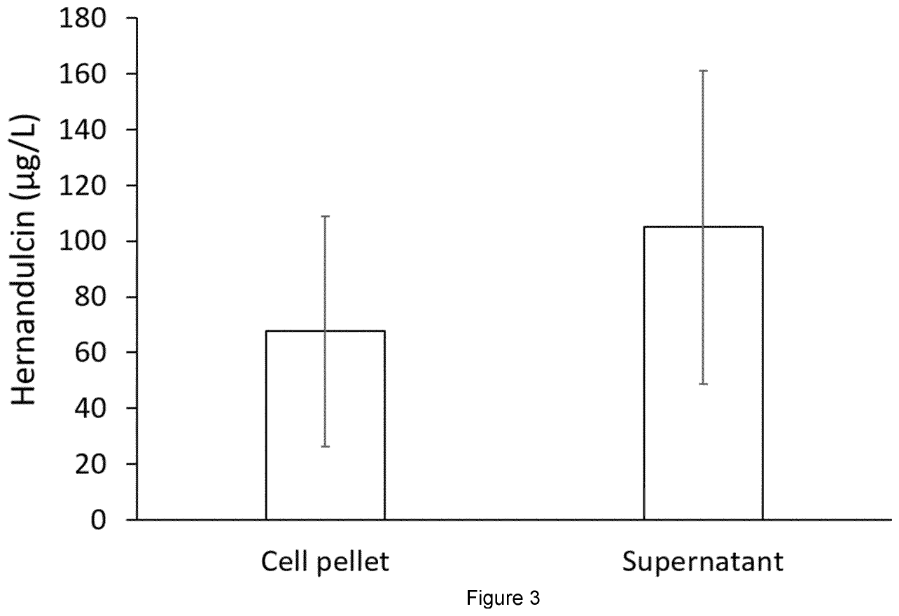

The presence of hemandulcin was demonstrated in the cultivation supernatant and cell phase extract of the HRN1-producer by LC-MS analysis (FIG. 2). Hemandulcin was quantified at 67.6±41.2 μg/L in the cell phase based on three replicates and at 104.9±56.3 μg/L in the supernatant (liquid phase) based on two replicates (FIG. 3, Table 4). Hemandulcin could not be detected in the supernatant of the parental strain ST10274.

TABLE 4
Presence of hernandulcin in engineered yeast supernatant or cell phase.

| | | | Hernandulcin |
|---|---|---|---|
| Strain number | Genotype | Phase | (μg/L) |
| ST10274 | ST9149 + LdTPS8 + LdCPR1 | Supernatant | nd |
| ST11474 (HRN1- | ST9149 + LdTPS8 + LdCPR1 + | Supernatant | 104.9336 ± |
| producer) | AtATR2 + NtEAH | | 56.3073* |
| ST11474 (HRN1- | ST9149 + LdTPS8 + LdCPR1 + | Cell | 67.5906 ± |
| producer) | AtATR2 + NtEAH | phase | 41.2332 |

Averages and standard deviations are based on three replicates or two replicates (marked with *). nd: not detected.

The genes LdTPS8, LdCPR1, AtATR2 and NtEAH were co-expressed in a strain (ST6512) without improved mevalonate (MVA) pathway flux generating the HRN2-producer (Table 3).

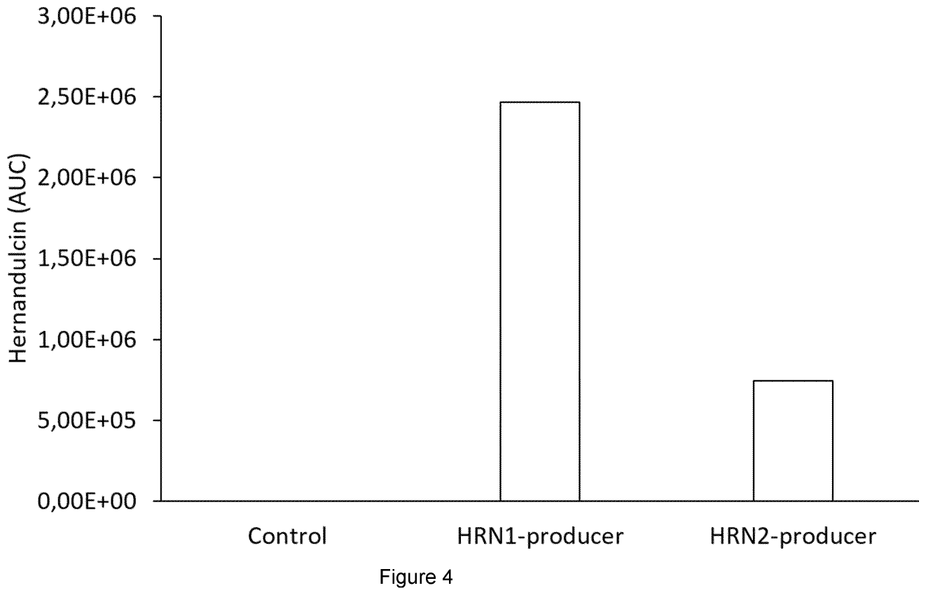

Hemandulcin [M+H−H₂O]⁺ was detected in the supernatant of the HRN2-producer by LC-MS analysis (FIG. 4, Table 5).

TABLE 5
Hernandulcin production by engineered yeast cells
with or without MVA-pathway optimization.

| | | Hernandulcin |
|---|---|---|
| | | [M + H − |
| Strain number | Genotype | H₂O]⁺ (AU) |
| ST10274 | ST9149 + LdTPS8 + LdCPR1 | nd |
| ST10275 | ST6512 + LdTPS8 + LdCPR1 | nd |
| ST11474 (HRN1- | ST9149 + LdTPS8 + LdCPR1 + | 2464413.521 |
| producer) | AtATR2 + NtEAH | |
| ST11669 (HRN2- | ST6512 + LdTPS8 + LdCPR1 + | 745117.6919 |
| producer) | AtATR2 + NtEAH | |

The supernatant samples were analyzed by LC-MS. Values are based on single replicates. nd: not detected.

The genes LdTPS8 and NtEAH were co-expressed with or without AtATR2 in ST6512 to test whether cytochrome P450 reductase was essential for hemandulcin production in Y. lipolytica. Hemandulcin could only be detected in supernatant samples by LC-MS analysis when LdTPS8, AtATR2, and NtEAH were co-expressed (Table 6).

TABLE 6
Hernandulcin production by engineered yeast cells with or without AtATR2-expression.

| | | | Hernandulcin | Hernandulcin | Hernandulcin |
|---|---|---|---|---|---|
| Strain | | Hernandulcin | [M + H]⁺ | [M + H − H₂O]⁺ | C8H13 |
| number | Genotype | (μM) | (AU) | (AU) | fragment (AU) |
| ST6512 | Parental | nd | nd | nd | nd |
| | strain | | | | |
| ST12430 | ST6512 + | nd | nd | nd | nd |
| | LdTPS8 | | | | |
| ST12433 | ST12430 + | nd | nd | nd | nd |
| | NtEAH | | | | |
| ST12439 | ST12430 + | 0.3344 ± | 147,917 ± | 937,610 ± | 403,017 ± |
| | NtEAH + | 0.0538* | 53,784* | 127,109 | 48,359 |
| | AtATR2 | | | | |

The supernatant samples were analyzed by LC-MS. Averages and standard deviations are based on three replicates or two replicates (marked with *). nd: not detected.

Sequences related to NtEAH from Solanum lycopersicum (SIEAH, SEQ ID NO 9), Datura stramonium (DsEAH, SEQ ID NO 11), S. commersonii (ScEAH, NCBI accession number. KAG5574796.1), Capsicum chinense (CcEAH, SEQ ID NO 13), C. annuum (CaEAH, SEQ ID NO 15), C. baccatum (CbEAH, NCBI accession number. PHT58219.1), and Hyoscyamus muticus (HmHPO, Uniprot accession: A6YIH8 (C7D55_HYOMU)) were co-expressed with LdTPS8 and AtATR2 in ST9149 (Table 3). Expression of DsEAH led to hemandulcin production, and hemandulcin [M+H−H₂O]⁺ was detected when SIEAH was expressed which suggested that small amounts of hemandulcin was produced (Table 7). However, since only the ion fragment [C₈H₁₃]⁺ was detected for engineered yeast cells expressing CcEAH and CaEAH, suggesting that low amounts of hemandulcin were produced by these strains.

TABLE 7
Hernandulcin production by engineered yeast cells co-expressing
NtEAH-related genes together with LdTPS8 and AtATR2.

| | | | | | Hernandulcin |
|---|---|---|---|---|---|
| | | | Hernandulcin | Hernandulcin | [C₈H₁₃]⁺ |
| Strain | | Hernandulcin | [M + H]⁺ | [M + H − H₂O]⁺ | fragment |
| number | Genotype | (μM) | (AU) | (AU) | (AU) |
| ST12475 | ST9149 + | nd | nd | nd | nd |
| | LdTPS8 + | | | | |
| | AtATR2 | | | | |
| ST12476 | ST12475 + | 0.6616 ± | 475,112 ± | 3,095,388 ± | 1,525,688 ± |
| | NtEAH | 0.0620 | 62,044 | 356,946 | 160,149 |
| ST12477 | ST12475 + | nd | nd | 250,445 ± | 678,875 ± |
| | SIEAH | | | 40,250 | 85,353 |
| ST12478 | ST12475 + | 0.4127 ± | 226,250 ± | 1,502,119 ± | 1,346,511 ± |
| | DsEAH | 0.0328 | 32,848 | 200,112 | 258,926 |
| ST12479 | ST12475 + | nd | nd | nd | nd |
| | ScEAH | | | | |
| ST12480 | ST12475 + | nd | nd | nd | 186,362 ± |
| | CcEAH | | | | 15,365 |
| ST12481 | ST12475 + | nd | nd | nd | 87,027 ± |
| | CaEAH | | | | 10,667 |
| ST12482 | ST12475 + | nd | nd | nd | nd |
| | CbEAH | | | | |
| ST12483 | ST12475 + | nd | nd | nd | nd |
| | HmHPO | | | | |

The supernatant samples were analyzed by LC-MS. Averages and standard deviations are based on three replicates. nd; not detected.

These results demonstrate that hemandulcin can be produced by engineered Y. lipolytica strains with or without prior engineering for precursor improvement.

### Sequence Overview

SEQ ID NO 1: LdTPS8 from Lippia dulcis (Phyla scarberrima), amino acid
SEQ ID NO 2: NtEAH from Nicotana tabacum, CYP71D20, Uniprot accession: Q94FM7 (C71DK_TOBAC), amino acid
SEQ ID NO 3: AtATR2 from Arabidopsis thaliana, accession number NP_194750.1, amino acid
SEQ ID NO 4: LdTPS8 from Lippia dulcis (Phyla scarberrima), nucleic acid, codon-optimised for Y. lipolytica
SEQ ID NO 5: NtEAH from Nicotana tabacum, nucleic acid, codon-optimised for Y. lipolytica
SEQ ID NO 6: AtATR2 from Arabidopsis thaliana, nucleic acid, codon-optimised for Y. lipolytica
SEQ ID NO 7: LdCPR1 from Lippia dulcis (Phyla scarberrima), amino acid
SEQ ID NO 8: LdCPR1 from Lippia dulcis (Phyla scarberrima), nucleic acid, codon-optimised for Y. lipolytica
SEQ ID NO 9: SIEAH from Solanum lycopersicum, amino acid, accession number XP_004249520.2
SEQ ID NO 10: SIEAH from Solanum lycopersicum, nucleic acid, codon-optimised for Y. lipolytica
SEQ ID NO 11: DsEAH from Datura stramonium, amino acid, accession number MCD7452234.1
SEQ ID NO 12: DsEAH from Datura stramonium, nucleic acid, codon-optimised for Y. lipolytica
SEQ ID NO 13: CcEAH from Capsicum chinense, amino acid, accession number-PHT99583.1
SEQ ID NO 14: CcEAH from Capsicum chinense, nucleic acid, codon-optimised for Y. lipolytica
SEQ ID NO 15: CaEAH from Capsicum annuum, amino acid, accession number-XP_016549742.1,
SEQ ID NO 16: CaEAH from Capsicum annuum, nucleic acid, codon-optimised for Y. lipolytica

### REFERENCES

https://iubmb.qmul.ac.uk/misc/naseq.html; NC-IUB, Eur J Biochem (1985) 1;150(1):1-5.
Arnesen et al., 2022. Engineering of Yarrowia lipolytica for the production of plant triterpenoids: Asiatic, madecassic, and arjunolic acid. Metab. Eng. Commun. 14, e00197.
Arnesen et al., 2020. Yarrowia lipolytica Strains Engineered for the Production of Terpenoids. Front. Bioeng. Biotechnol. 8, 1-14.
Attia et al., 2012. Molecular cloning and characterization of (+)-epi-α-bisabolol synthase, catalyzing the first step in the biosynthesis of the natural sweetener, hemandulcin, in Lippia dulcis. Arch. Biochem. Biophys. 527, 37-44.
De Oliveira et al., 2012. Supercritical fluid extraction of hemandulcin from Lippia dulcis Trev. J. Supercrit. Fluids 63, 161-168.
Holkenbrink et al., 2018. EasyCloneYALI: CRISPR/Cas9-Based Synthetic Toolbox for Engineering of the Yeast Yarrowia lipolytica. Biotechnol. J. 13, 1-8.
Istiandari et. al 2021. Comparative Analysis of NADPH-Cytochrome P450 Reductases From Legumes for Heterologous Production of Triterpenoids in Transgenic Saccharomyces cerevisiae. Front. Plant Sci. 12, 1-18
Jung et al., 2002. Synthesis of (+)-hemandulcin and (+)-epihernandulcin. Tetrahedron Lett. 43, 4721-4722.
Kildegaard et al., 2021. Tailored biosynthesis of gibberellin plant hormones in yeast. Metab. Eng. 66, 1-11.
Luo et al., 2020. Enhancing isoprenoid synthesis in Yarrowia lipolytica by expressing the isopentenol utilization pathway and modulating intracellular hydrophobicity. Metab. Eng. 61, 344-351.
Marella et al., 2020. A single-host fermentation process for the production of flavor lactones from non-hydroxylated fatty acids. Metab. Eng. 61, 427-436.
Ralston et al., 2001. Cloning, heterologous expression, and functional characterization of 5-epi-Aristolochene-1, 3-Dihydroxylase from tobacco (Nicotiana tabacum). Arch. Biochem. Biophys. 393, 222-235.
Sarrade-Loucheur et al., 2020. Synthetic derivatives of (+)-epi-α-bisabolol are formed by mammalian cytochromes P450 expressed in a yeast reconstituted pathway. ACS Synth. Biol.
Takahashi et al., 2007. Functional Characterization of Premnaspirodiene Oxygenase, a Cytochrome P450 Catalyzing Regio- And Stereo-specific Hydroxylations of Diverse Sesquiterpene Substrates. J. Biol. Chem. 282, 31744-31754.
Villa-Ruano et al., 2021. Hemandulcin Production in Cell Suspensions of Phyla Scaberrima: Exploring Hemandulcin Accumulation through Physical and Chemical Stimuli. Chem. Biodivers.

### Items 1

1. A yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol, preferably a heterologous (+)-epi-alpha-bisabolol synthase;
ii. at least one cytochrome P450 enzyme capable of converting (+)-epi-α-bisabolol into hemandulcin, preferably a heterologous cytochrome P450 enzyme such as Nicotiana tabacum 5-epi-aristolocene dihydroxylase (NtEAH) as set forth in SEQ ID NO 2 or a functional variant thereof having at least 65% identity, homology or similarity thereto; and
iii. optionally at least one cytochrome P450 reductase, preferably a heterologous cytochrome P450 reductase,
whereby said yeast cell is capable of producing hemandulcin and/or one or more derivatives thereof.

2. The yeast cell according to item 1, wherein:
i. the at least one (+)-epi-alpha-bisabolol synthase is a (+)-epi-alpha-bisabolol synthase with EC 4.2.3.138;
ii. the at least one cytochrome P450 enzyme is a cytochrome P450 oxygenase belonging to EC 1.14.14.-; and/or
iii. the at least one cytochrome P450 reductase is a cytochrome P450 reductase with EC 1.6.2.4.

3. The yeast cell according to any one of the preceding items, wherein:
i. the at least one (+)-epi-alpha-bisabolol synthase is a Lippia (+)-epi-alpha-bisabolol synthase, such as a Lippia dulcis (+)-epi-alpha-bisabolol synthase, preferably the Lippia dulcis terpene synthase 8 (LdTPS8) as set forth in SEQ ID NO 1 or a functional variant thereof having at least 70% identity, homology or similarity thereto;
ii. the at least one cytochrome P450 enzyme is a 5-epi-aristolocene dihydroxylase, such as Nicotiana 5-epi-aristolocene dihydroxylase, preferably a Nicotiana tabacum 5-epi-aristolocene dihydroxylase, more preferably the Nicotiana tabacum 5-epi-aristolocene dihydroxylase (NtEAH) as set forth in SEQ ID NO 2 or a functional variant thereof having at least 65% identity, homology or similarity thereto; and/or
iii. the at least one cytochrome P450 reductase is a plant cytochrome P450 reductase, such as an Arabidopsis cytochrome P450 reductase, such as an Arabidopsis thaliana cytochrome P450 reductase, such as the Arabidopsis thaliana cytochrome P450 reductase ATR2 (AtATR2) as set forth in SEQ ID NO 3 or a functional variant thereof having at least 70% identity, homology or similarity thereto.

4. The yeast cell according to any one of the preceding items, wherein the yeast cell expresses at least one further cytochrome P450 reductase, preferably a heterologous cytochrome P450 reductase, optionally wherein said at least one further cytochrome P450 reductase is a plant cytochrome P450 reductase such as a Lippia cytochrome P450 reductase, such as a Lippia dulcis cytochrome P450 reductase, preferably the Lippia dulcis cytochrome P450 reductase LdCPR1 as set forth in SEQ ID NO 7, or a functional variant thereof having at least 70% identity, homology or similarity thereto.

5. The yeast cell according to any one of the preceding items, wherein the yeast cell is a non-pathogenic yeast cell.

6. The yeast cell according to any one of the preceding items, wherein said yeast cell belongs to a genus selected from Saccharomyces, Pichia, Yarrowia, Kluyveromyces, Candida, Rhodotorula, Rhodospoidium, Cryptococcus, Tichosporon and Lipomyces, optionally wherein the yeast cell belongs to a species selected from Saccharomyces cerevisiae, Saccharomyces boulardi, Komagetella phaffi (Pichia pastoris), Kluyveromyces marxianus, Cryptococcus albidus, Lipomyces lipofera, Lipomyces starkeyi, Rhodospondium toruloides, Rhodotorula glutinis, Trichosporon pullulan and Yarrowia lipolytica, preferably the yeast cell is a Yarrowia lipolytica cell.

7. The yeast cell according to any one of the preceding items, wherein the yeast cell is further modified by:
i. having a mutation resulting in increased activity of one or more of HMG, ERG12, ACL, IDI, and ERG20 or a functional variant thereof having at least 70% identity, homology or similarity to any of said polypeptides;
ii. overexpressing Salmonella enterica ACSM^{L641P} or a functional variant thereof having at least 70% identity, homology or similarity thereto; and/or
iii. having a mutation resulting in reduced activity of SQS such as ERG9 and/or SQS1 or a functional variant thereof having at least 70% identity, homology or similarity thereto;
   whereby production of farnesyl diphosphate (FPP) is improved.

8. The yeast cell according to item 7, wherein the mutation resulting in increased activity of a polypeptide comprises overexpression of a gene encoding said polypeptide.

9. The yeast cell according to item 7, wherein the mutation resulting in reduced activity of SQS comprises modifying the promoter of SQS such as by replacing the SQS promoter sequence, fragments thereof or a homologue thereof having at least 70% identity, homology or similarity to the SQS promoter sequence, for the nucleic acid sequence of the ERG11 promoter or a homologue thereof having at least 70% identity, homology or similarity thereto.

10. The yeast cell according to items 7 to 9, wherein the mutation resulting in reduced activity of a polypeptide comprises down-regulation of a gene encoding said polypeptide and/or mutation of said polypeptide such as a loss-of-function mutation.

11. The yeast cell according to any one of items 7 to 10, wherein the polypeptides are native to the yeast cell or are non-native to the yeast cell or a combination of native and non-native.

12. The yeast cell according to any one of the preceding items, wherein hemandulcin is produced with at titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

13. The yeast cell according to any one of the preceding items, wherein the yeast cell comprises:
i. a nucleic acid encoding the at least one (+)-epi-alpha-bisabolol synthase, preferably wherein said nucleic acid comprises or consists of LdTPS8 as set forth in SEQ ID NO 4, or a homologue thereof having at least 70% identity, homology or similarity thereto;
ii. a nucleic acid encoding the at least one cytochrome P450 enzyme, preferably wherein said nucleic acid comprises or consists of NtEAH as set forth in SEQ ID NO 5, or a homologue thereof having at least 65% identity, homology or similarity thereto; and
iii. optionally a nucleic acid encoding the at least one plant cytochrome P450 reductase, preferably wherein said nucleic acid comprises or consists of AtATR2 as set forth in SEQ ID NO 6, or a homologue thereof having at least 70% identity, homology or similarity thereto; and
iv. optionally a nucleic acid encoding the at least one further cytochrome P450 reductase, preferably wherein said nucleic acid comprises or consists of LdCPR1 as set forth in SEQ ID NO 8, or a homologue thereof having at least 70% identity, homology or similarity thereto.

14. The yeast cell according to any one of the preceding items, wherein one or more of the at least one (+)-epi-alpha-bisabolol synthase nucleic acid, the at least one cytochrome P450 enzyme nucleic acid, the at least one cytochrome P450 reductase nucleic acid and/or the at least one further P450 reductase nucleic acid is codon-optimised.

15. An expression system for expression in a yeast cell, comprising:
i. a nucleic acid encoding at least one (+)-epi-alpha-bisabolol synthase (EC 4.2.3.138), preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto;
ii. a nucleic acid encoding at least one cytochrome P450 enzyme, preferably a heterologous cytochrome P450 enzyme such as Nicotiana tabacum 5-epi-aristolocene dihydroxylase (NtEAH) as set forth in SEQ ID NO 2 or a functional variant thereof having at least 65% identity, homology or similarity thereto; and
iii. optionally a nucleic acid encoding at least one cytochrome P450 reductase (EC 1.6.2.4), preferably a heterologous cytochrome P450 reductase such as Arabidopsis thaliana cytochrome P450 reductase ATR2 (AtATR2) (EC 1.6.2.4) as set forth in SEQ ID NO 3 or a functional variant thereof having at least 70% identity, homology or similarity thereto; and
iv. optionally a nucleic acid encoding at least one further cytochrome P450 reductase, preferably a heterologous cytochrome P450 reductase such as Lippia dulcis cytochrome P450 reductase LdCPR1 (EC 1.6.2.4) as set forth in SEQ ID NO 7 or a functional variant thereof having at least 70% identity, homology or similarity thereto.

16. The expression system according to item 15, wherein:
i. the nucleic acid encoding the at least one (+)-epi-alpha-bisabolol synthase comprises or consists of LdTPS8 as set forth in SEQ ID NO 4 or a homologue thereof having at least 70% identity, homology or similarity thereto;
ii. the nucleic acid encoding the at least one cytochrome P450 enzyme comprises or consists of NtEAH as set forth in SEQ ID NO 5 or a homologue thereof having at least 65% identity, homology or similarity thereto; and
iii. optionally the nucleic acid encoding the at least one cytochrome P450 reductase comprises or consists of AtATR2 as set forth in SEQ ID NO 6 or a homologue thereof having at least 70% identity, homology or similarity thereto; and
iv. optionally the nucleic acid encoding the at least one further cytochrome P450 reductase comprises or consists of LdCPR1 as set forth in SEQ ID NO 8 or a homologue thereof having at least 70% identity, homology or similarity thereto.

17. The expression system according to any one of items 15 to 16, wherein the nucleic acids further comprises one or more promoters.

18. The expression system according to any one of items 15 to 17, wherein one or more of the at least one (+)-epi-alpha-bisabolol synthase nucleic acid, the at least one cytochrome P450 enzyme nucleic acid, the at least one cytochrome P450 reductase nucleic acid and/or the at least one further P450 reductase nucleic acid is codon-optimised.

19. The expression system according to any one of items 15 to 19, wherein the yeast cell is as defined in any one of items 1 to 14.

20. The yeast cell according to any one of items 1 to 14, said yeast cell comprising the expression system according to any one of items 15 to 19, whereby said yeast cell is capable of producing hemandulcin and/or one or more derivatives thereof.

21. A method for producing hemandulcin and/or one or more derivatives thereof in a yeast cell, said method comprising the steps of:
i. providing a yeast cell according to any one of items 1 to 14 or 20; and
ii. incubating said yeast cell in a medium,
whereby hemandulcin and/or one or more derivatives thereof is produced.

22. The method according to item 21, wherein the yeast cell is as defined in anyone of items 1 to 14 or 20.

23. The method according to any one of items 21 to 22, wherein the yeast cell comprises an expression system as defined in any one of items 15 to 19.

24. The method according to any of items one of the preceding items, wherein hemandulcin and/or the one or more derivatives thereof is produced with at titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

25. The method according to any one of the preceding claims 9 to 10, further comprising the steps of:
i. recovering the hemandulcin and/or the one or more derivatives thereof; and
ii. optionally converting said hemandulcin and/or the one or more derivatives thereof to one or more further derivatives thereof; and/or
iii. formulating said hemandulcin and/or the one or more derivatives thereof in a composition.

26. A composition comprising hemandulcin and/or one or more derivatives thereof obtained by the method according to any one of items 21 to 25.

27. Hemandulcin and/or one or more derivatives thereof obtained by the method according to any one of items 21 to 25.

28. Use of a Nicodana cytochrome P450 enzyme, preferably a Nicodana tabacum cytochrome P450 enzyme, in a method for producing hemandulcin and/or one or more derivatives, optionally wherein the Nicodana tabacum cytochrome P450 enzyme is NtEAH as set forth in SEQ ID NO 2, or a functional variant thereof having at least 65% identity, homology or similarity thereto.

29. The use according to item 28, wherein the polypeptide comprises the sequence as set forth in SEQ ID NO 2, with the exception that at the most 51 residues are mutated.

30. The use according to any one of items 28 to 29, said use comprising expressing the Nicodana cytochrome P450 enzyme, preferably the Nicodana tabacum cytochrome P450 enzyme, optionally wherein the Nicotiana tabacum cytochrome P450 enzyme is NtEAH as set forth in SEQ ID NO 2, or functional variants thereof having at least 65% identity, homology or similarity thereto in a yeast cell, preferably wherein said yeast cell is as defined in any one of items 1 to 14 or 20.

31. The use according to any one of items 28 to 30, wherein said method is as defined in any one of items 21 to 25.

### Items 2

1. A yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol, preferably a heterologous (+)-epi-alpha-bisabolol synthase;
ii. at least one cytochrome P450 enzyme capable of converting (+)-epi-α-bisabolol into hemandulcin, preferably a heterologous cytochrome P450 enzyme such as Nicotana tabacum 5-epi-aristolocene dihydroxylase (NtEAH) as set forth in SEQ ID NO 2 or a functional variant thereof having at least 65% identity, homology or similarity thereto; and
iii. optionally at least one cytochrome P450 reductase, preferably a heterologous cytochrome P450 reductase,
   whereby said yeast cell is capable of producing hemandulcin and/or one or more derivatives thereof.

2. The yeast cell according to any one of the preceding items, wherein:
i. the at least one (+)-epi-alpha-bisabolol synthase is a Lippia (+)-epi-alpha-bisabolol synthase, such as a Lippia dulcis (+)-epi-alpha-bisabolol synthase, preferably the Lippia dulcis terpene synthase 8 (LdTPS8) as set forth in SEQ ID NO 1 or a functional variant thereof having at least 70% identity, homology or similarity thereto;
ii. the at least one cytochrome P450 enzyme is a 5-epi-aristolocene dihydroxylase, such as Nicotana 5-epi-aristolocene dihydroxylase, preferably a Nicotana tabacum 5-epi-aristolocene dihydroxylase, more preferably the Nicotana tabacum 5-epi-aristolocene dihydroxylase (NtEAH) as set forth in SEQ ID NO 2 or a functional variant thereof having at least 65% identity, homology or similarity thereto; and
iii. the at least one cytochrome P450 reductase is a plant cytochrome P450 reductase, such as an Arabidopsis cytochrome P450 reductase, such as an Arabidopsis thaliana cytochrome P450 reductase, such as the Arabidopsis thaliana cytochrome P450 reductase ATR2 (AtATR2) as set forth in SEQ ID NO 3 or a functional variant thereof having at least 70% identity, homology or similarity thereto.

3. The yeast cell according to any one of the preceding items, wherein the yeast cell expresses at least one further cytochrome P450 reductase, preferably a heterologous cytochrome P450 reductase, optionally wherein said at least one further cytochrome P450 reductase is a plant cytochrome P450 reductase such as a Lippia cytochrome P450 reductase, such as a Lippia dulcis cytochrome P450 reductase, preferably the Lippia dulcis cytochrome P450 reductase LdCPR1 (EC 1.6.2.4) as set forth in SEQ ID NO 7, or a functional variant thereof having at least 70% identity, homology or similarity thereto.

4. The yeast cell according to any one of the preceding items, wherein said yeast cell belongs to a genus selected from Saccharomyces, Pichia, Yarrowia, Kluyveromyces, Candida, Rhodotorula, Rhodosporidium, Cryptococcus, Trichosporon and Lipomyces, optionally wherein the yeast cell belongs to a species selected from Saccharomyces cerevisiae, Saccharomyces boulardi, Komagetella phaffi (Pichia pastoris), Kluyveromyces marxianus, Cryptococcus albidus, Lipomyces lipofera, Lipomyces starkeyi, Rhodospondium toruloides, Rhodotorula glutinis, Trichosporon pullulan and Yarrowia lipolytica, preferably the yeast cell is a Yarrowia lipolytica cell.

5. The yeast cell according to any one of the preceding items, wherein the yeast cell is further modified by;
iv. having a mutation resulting in increased activity of one or more of HMG, ERG12, ACL, IDI, and ERG20 or a functional variant thereof having at least 70% identity, homology or similarity to any of said polypeptides;
v. overexpressing Salmonella enterica ACS^{L641P} or a functional variant thereof having at least 70% identity, homology or similarity thereto; and/or
vi. having a mutation resulting in reduced activity of SQS such as ERG9 and/or SQS1 or a functional variant thereof having at least 70% identity, homology or similarity thereto, optionally the mutation resulting in reduced activity of SQS comprises modifying the promoter of SQS such as by replacing the SQS promoter sequence, fragments thereof or a homologue thereof having at least 70% identity, homology or similarity to the SQS promoter sequence, for the nucleic acid sequence of the ERG11 promoter or a homologue thereof having at least 70% identity, homology or similarity thereto,
   whereby production of farnesyl diphosphate (FPP) is improved.

6. The yeast cell according to any one of the preceding items, wherein hemandulcin is produced with at titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

7. An expression system for expression in a yeast cell, comprising:
i. a nucleic acid encoding at least one (+)-epi-alpha-bisabolol synthase (EC 4.2.3.138), preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto;
ii. a nucleic acid encoding at least one cytochrome P450 enzyme, preferably a heterologous cytochrome P450 enzyme such as Nicotiana tabacum 5-epi-aristolocene dihydroxylase (NtEAH) as set forth in SEQ ID NO 2 or a functional variant thereof having at least 65% identity, homology or similarity thereto; and
iii. optionally a nucleic acid encoding at least one cytochrome P450 reductase (EC 1.6.2.4), preferably a heterologous cytochrome P450 reductase such as Arabidopsis thaliana cytochrome P450 reductase ATR2 (AtATR2) (EC 1.6.2.4) as set forth in SEQ ID NO 3 or a functional variant thereof having at least 70% identity, homology or similarity thereto; and
iv. optionally a nucleic acid encoding at least one further cytochrome P450 reductase, preferably a heterologous cytochrome P450 reductase such as Lippia dulcis cytochrome P450 reductase LdCPR1 as set forth in SEQ ID NO 7 or a functional variant thereof having at least 70% identity, homology or similarity thereto.

8. The yeast cell according to any one of items 1 to 6, said yeast cell comprising the expression system according to item 7, whereby said yeast cell is capable of producing hemandulcin and/or one or more derivatives thereof.

9. A method for producing hemandulcin and/or one or more derivatives thereof in a yeast cell, said method comprising the steps of:
i. providing a yeast cell according to any one of items 1 to 6 or 8; and
ii. incubating said yeast cell in a medium,
whereby hemandulcin and/or one or more derivatives thereof is produced.

10. The method according to item 9, wherein hemandulcin and/or the one or more derivatives thereof is produced with at titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

11. The method according to any one of the preceding items 9 to 10, further comprising the steps of:
i. recovering the hemandulcin and/or the one or more derivatives thereof; and
ii. optionally converting said hemandulcin and/or the one or more derivatives thereof to one or more further derivatives thereof; and/or
iii. formulating said hemandulcin and/or the one or more derivatives thereof in a composition.

12. A composition comprising hemandulcin and/or one or more derivatives thereof obtained by the method according to any one of items 9 to 11.

13. Use of a Nicotiana cytochrome P450 enzyme, preferably a Nicotiana tabacum cytochrome P450 enzyme, in a method for producing hemandulcin and/or one or more derivatives, optionally wherein the Nicotiana tabacum cytochrome P450 enzyme is NtEAH as set forth in SEQ ID NO 2, or a functional variant thereof having at least 65% identity, homology or similarity thereto.

14. The use according to item 13, said use comprising expressing the Nicotiana cytochrome P450 enzyme, preferably the Nicotiana tabacum cytochrome P450 enzyme, optionally wherein the Nicotiana tabacum cytochrome P450 enzyme is NtEAH as set forth in SEQ ID NO 2, or functional variants thereof having at least 65% identity, homology or similarity thereto in a yeast cell, preferably wherein said yeast cell is as defined in any one of items 1 to 6 or 8.

15. The use according to any one of items 13 to 14, wherein said method is as defined in any one of items 9 to 11.

1. A yeast cell capable of producing hemandulcin and/or one or more derivatives thereof, said yeast cell expressing:
i. at least one (+)-epi-alpha-bisabolol synthase capable of converting farnesyl diphosphate into (+)-epi-α-bisabolol, preferably a heterologous (+)-epi-alpha-bisabolol synthase;
ii. at least one plant cytochrome P450 enzyme capable of converting (+)-epi-α-bisabolol into hemandulcin, preferably a heterologous plant cytochrome P450 enzyme such as Nicotiana tabacum 5-epi-aristolocene dihydroxylase (NtEAH) as set forth in SEQ ID NO 2, DsEAH as set forth in SEQ ID NO 11, or a functional variant thereof having at least 65% identity, homology or similarity to any of the aforementioned; and
iii. at least one cytochrome P450 reductase, preferably a heterologous cytochrome P450 reductase,
whereby said yeast cell is capable of producing hemandulcin and/or one or more derivatives thereof.

2. The yeast cell according to claim 1, wherein:
i. the at least one (+)-epi-alpha-bisabolol synthase is a (+)-epi-alpha-bisabolol synthase with EC 4.2.3.138;
ii. the at least one cytochrome P450 enzyme is a cytochrome P450 oxygenase belonging to EC 1.14.14.-; and/or
iii. the at least one cytochrome P450 reductase is a cytochrome P450 reductase with EC 1.6.2.4.

3. The yeast cell according to any one of the preceding claims, wherein the at least one (+)-epi-alpha-bisabolol synthase is a Lippia (+)-epi-alpha-bisabolol synthase, such as a Lippia dulcis (+)-epi-alpha-bisabolol synthase, preferably the Lippia dulcis terpene synthase 8 (LdTPS8) as set forth in SEQ ID NO 1 or a functional variant thereof having at least 70% identity, homology or similarity thereto.

4. The yeast cell according to any one of the preceding claims, wherein the at least one plant cytochrome P450 enzyme is native to an organism of a genus selected from Nicotiana, Solanum, Datura, and Capsicum, such as Nicotana tabacum, Solanum lycopersicum, Datura stramonium, Capsicum chinense and C. annuum, optionally wherein the plant cytochrome P450 enzyme is NtEAH (SEQ ID NO 2), SIEAH (SEQ ID NO 9), DsEAH (SEQ ID NO 11), CcEAH (SEQ ID NO 13) or CaEAH (SEQ ID NO 15), or a functional variant thereof having at least 65% identity, homology or similarity to any of the aforementioned.

5. The yeast cell according to any one of the preceding claims, wherein the at least one plant cytochrome P450 enzyme is a 5-epi-aristolocene dihydroxylase, such as Nicotiana 5-epi-aristolocene dihydroxylase, preferably a Nicotiana tabacum 5-epi-aristolocene dihydroxylase, more preferably the Nicotiana tabacum 5-epi-aristolocene dihydroxylase (NtEAH) as set forth in SEQ ID NO 2 or a functional variant thereof having at least 65% identity, homology or similarity thereto.

6. The yeast cell according to any one of the preceding claims, wherein the at least one plant cytochrome P450 enzyme is a Datura cytochrome P450 enzyme, preferably a Datura stramonium cytochrome P450 enzyme, more preferably DsEAH as set forth in SEQ ID NO 11, or a functional variant thereof having at least 65% identity, homology or similarity thereto.

7. The yeast cell according to any one of the preceding claims, wherein the at least one plant cytochrome P450 enzyme is a premnaspirodiene oxygenase-like protein, such as a Solanum premnaspirodiene oxygenase-like protein, preferably a Solanum lycopersicum premnaspirodiene oxygenase-like protein, more preferably the Solanum lycopersicum premnaspirodiene oxygenase-like protein (SIEAH) as set forth in SEQ ID NO 9, or a functional variant thereof having at least 65% identity, homology or similarity thereto.

8. The yeast cell according to any one of the preceding claims, wherein the at least one plant cytochrome P450 enzyme is a cytochrome P450 71D7-like protein, such as a Capsicum cytochrome P450 71D7-like protein, preferably a Capsicum annuum cytochrome P450 71D7-like protein, more preferably the Capsicum annuum cytochrome P450 71D7-like protein (CaEAH) as set forth in SEQ ID NO 15, or a functional variant thereof having at least 65% identity, homology or similarity thereto.

9. The yeast cell according to any one of the preceding claims, wherein the at least one plant cytochrome P450 enzyme is a Capsicum cytochrome P450 enzyme, preferably a Capsicum chinense cytochrome P450 enzyme, more preferably the CcEAH as set forth in SEQ ID NO 13, or a functional variant thereof having at least 65% identity, homology or similarity thereto.

10. The yeast cell according to any one of the preceding claims, wherein the at least one cytochrome P450 reductase is a plant cytochrome P450 reductase, such as an Arabidopsis cytochrome P450 reductase, such as an Arabidopsis thaliana cytochrome P450 reductase, such as the Arabidopsis thaliana cytochrome P450 reductase ATR2 (AtATR2) as set forth in SEQ ID NO 3 or a functional variant thereof having at least 70% identity, homology or similarity thereto.

11. The yeast cell according to any one of the preceding claims, wherein the yeast cell expresses at least one further cytochrome P450 reductase, preferably a heterologous cytochrome P450 reductase, optionally wherein said at least one further cytochrome P450 reductase is a plant cytochrome P450 reductase such as a Lippia cytochrome P450 reductase, such as a Lippia dulcis cytochrome P450 reductase, preferably the Lippia dulcis cytochrome P450 reductase LdCPR1 as set forth in SEQ ID NO 7, or a functional variant thereof having at least 70% identity, homology or similarity thereto.

12. The yeast cell according to any one of the preceding claims, wherein the yeast cell is a non-pathogenic yeast cell.

13. The yeast cell according to any one of the preceding claims, wherein said yeast cell belongs to a genus selected from Saccharomyces, Pichia, Yarrowia, Kluyveromyces, Candida, Rhodotorula, Rhodospondium, Cryptococcus, Trichosporon and Lipomyces, optionally wherein the yeast cell belongs to a species selected from Saccharomyces cerevisiae, Saccharomyces boulardi, Komagetella phaffi (Pichia pastoris), Kluyveromyces marxianus, Cryptococcus albidus, Lipomyces lipofera, Lipomyces starkeyi, Rhodospondium toruloides, Rhodotorula glutinis, Trichosporon pullulan and Yarrowia lipolytica, preferably the yeast cell is a Yarrowia lipolytica cell.

14. The yeast cell according to any one of the preceding claims, wherein the yeast cell is further modified by:
i. having a mutation resulting in increased activity of one or more of HMG, ERG12, ACL, IDI, and ERG20 or a functional variant thereof having at least 70% identity, homology or similarity to any of said polypeptides;
ii. overexpressing Salmonella enterica ACS^{L641P} or a functional variant thereof having at least 70% identity, homology or similarity thereto; and/or
iii. having a mutation resulting in reduced activity of SQS such as ERG9 and/or SQS1 or a functional variant thereof having at least 70% identity, homology or similarity thereto;
whereby production of farnesyl diphosphate (FPP) is improved.

15. The yeast cell according to claim 14, wherein the mutation resulting in increased activity of a polypeptide comprises overexpression of a gene encoding said polypeptide.

16. The yeast cell according to claim 14, wherein the mutation resulting in reduced activity of SQS comprises modifying the promoter of SQS such as by replacing the SQS promoter sequence, fragments thereof or a homologue thereof having at least 70% identity, homology or similarity to the SQS promoter sequence, for the nucleic acid sequence of the ERG11 promoter or a homologue thereof having at least 70% identity, homology or similarity thereto.

17. The yeast cell according to claims 14 to 16, wherein the mutation resulting in reduced activity of a polypeptide comprises down-regulation of a gene encoding said polypeptide and/or mutation of said polypeptide such as a loss-of-function mutation.

18. The yeast cell according to any one of claims 14 to 17, wherein the polypeptides are native to the yeast cell or are non-native to the yeast cell or a combination of native and non-native.

19. The yeast cell according to any one of the preceding claims, wherein hemandulcin is produced with at titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

20. The yeast cell according to any one of the preceding claims, wherein the yeast cell comprises:
i. a nucleic acid encoding the at least one (+)-epi-alpha-bisabolol synthase, preferably wherein said nucleic acid comprises or consists of LdTPS8 as set forth in SEQ ID NO 4, or a homologue thereof having at least 70% identity, homology or similarity thereto;
ii. a nucleic acid encoding the at least one cytochrome P450 enzyme, preferably wherein said nucleic acid comprises or consists of NtEAH as set forth in SEQ ID NO 5, DsEAH as set forth in SEQ ID NO 12, or homologues thereof having at least 65% identity, homology or similarity thereto; and
iii. a nucleic acid encoding the at least one plant cytochrome P450 reductase, preferably wherein said nucleic acid comprises or consists of AtATR2 as set forth in SEQ ID NO 6, or a homologue thereof having at least 70% identity, homology or similarity thereto; and
iv. optionally a nucleic acid encoding the at least one further cytochrome P450 reductase, preferably wherein said nucleic acid comprises or consists of LdCPR1 as set forth in SEQ ID NO 8, or a homologue thereof having at least 70% identity, homology or similarity thereto.

21. The yeast cell according to claim 20, wherein:
i. the nucleic acid encoding the at least one (+)-epi-alpha-bisabolol synthase comprises or consists of LdTPS8 as set forth in SEQ ID NO 4 or a homologue thereof having at least 70% identity, homology or similarity thereto;
ii. the nucleic acid encoding the at least one plant cytochrome P450 enzyme comprises or consists of
i. NtEAH as set forth in SEQ ID NO 5,
ii. SIEAH as set forth in SEQ ID NO 10,
iii. DsEAH as set forth in SEQ ID NO 12,
iv. CcEAH as set forth in SEQ ID NO 14,
v. CaEAH as set forth in SEQ ID NO 16,
or a homologue thereof having at least 65% identity, homology or similarity to any of the aforementioned; and
iii. the nucleic acid encoding the at least one cytochrome P450 reductase comprises or consists of AtATR2 as set forth in SEQ ID NO 6 or a homologue thereof having at least 70% identity, homology or similarity thereto.

22. The yeast cell according to any one of the preceding claims, wherein one or more of the at least one (+)-epi-alpha-bisabolol synthase nucleic acid, the at least one plant cytochrome P450 enzyme nucleic acid, the at least one cytochrome P450 reductase nucleic acid and/or the at least one further P450 reductase nucleic acid is codon-optimised.

23. An expression system for expression in a yeast cell, comprising:
i. a nucleic acid encoding at least one (+)-epi-alpha-bisabolol synthase (EC 4.2.3.138), preferably a heterologous (+)-epi-alpha-bisabolol synthase such as Lippia dulcis terpene synthase 8 (LdTPS8) as set forth in SEQ ID NO 1 or a functional variant thereof having at least 70% identity, homology or similarity thereto;
ii. a nucleic acid encoding at least one plant cytochrome P450 enzyme, preferably a heterologous plant cytochrome P450 enzyme such as Nicotiana tabacum 5-epi-aristolocene dihydroxylase (NtEAH) as set forth in SEQ ID NO 2, DsEAH as set forth in SEQ ID NO 11, or a functional variant thereof having at least 65% identity, homology or similarity to any of the aforementioned; and
iii. a nucleic acid encoding at least one cytochrome P450 reductase (EC 1.6.2.4), preferably a heterologous cytochrome P450 reductase, for example a plant cytochrome P450 reductase such as Arabidopsis thaliana cytochrome P450 reductase ATR2 (AtATR2) (EC 1.6.2.4) as set forth in SEQ ID NO 3 or a functional variant thereof having at least 70% identity, homology or similarity thereto.

24. The expression system according to claim 23, wherein:
i. the nucleic acid encoding the at least one (+)-epi-alpha-bisabolol synthase comprises or consists of LdTPS8 as set forth in SEQ ID NO 4 or a homologue thereof having at least 70% identity, homology or similarity thereto;
ii. the nucleic acid encoding the at least one plant cytochrome P450 enzyme comprises or consists of:
i. NtEAH as set forth in SEQ ID NO 5,
ii. SIEAH as set forth in SEQ ID NO 10,
iii. DsEAH as set forth in SEQ ID NO 12,
iv. CcEAH as set forth in SEQ ID NO 14,
v. CaEAH as set forth in SEQ ID NO 16,
or a homologue thereof having at least 65% identity, homology or similarity to any of the aforementioned; and
iii. the nucleic acid encoding the at least one cytochrome P450 reductase comprises or consists of AtATR2 as set forth in SEQ ID NO 6 or a homologue thereof having at least 70% identity, homology or similarity thereto.

25. The expression system according to any one of claims 23 to 24, further comprising a nucleic acid encoding at least one further cytochrome P450 reductase, preferably a heterologous cytochrome P450 reductase, optionally a plant cytochrome P450 reductase, such as Lippia dulcis cytochrome P450 reductase LdCPR1 (EC 1.6.2.4) as set forth in SEQ ID NO 7 or a functional variant thereof having at least 70% identity, homology or similarity thereto.

26. The expression system according to any one of claims 23 to 25, wherein the nucleic acids further comprises one or more promoters.

27. The expression system according to any one of claims 23 to 26, wherein one or more of the at least one (+)-epi-alpha-bisabolol synthase nucleic acid, the at least one plant cytochrome P450 enzyme nucleic acid, the at least one cytochrome P450 reductase nucleic acid and/or the at least one further cytochrome P450 reductase nucleic acid is codon-optimised.

28. The expression system according to any one of claims 23 to 27, wherein the yeast cell is as defined in any one of claims 1 to 22.

29. The yeast cell according to any one of claims 1 to 22, said yeast cell comprising the expression system according to any one of claims 23 to 28, whereby said yeast cell is capable of producing hemandulcin and/or one or more derivatives thereof.

30. A method for producing hemandulcin and/or one or more derivatives thereof in a yeast cell, said method comprising the steps of:
i. providing a yeast cell according to any one of claims 1 to 22 or 29; and
ii. incubating said yeast cell in a medium,
whereby hemandulcin and/or one or more derivatives thereof is produced.

31. The method according to claim 30, wherein the yeast cell is as defined in anyone of claims 1 to 22 or 29.

32. The method according to any one of claims 30 to 31, wherein the yeast cell comprises an expression system as defined in any one of claims 23 to 28.

33. The method according to any of claims 30 to 32, wherein hemandulcin and/or the one or more derivatives thereof is produced with at titer of at least 10 μg/L, such as at least 15 μg/L, such as at least 20 μg/L, such as at least 25 μg/L, such as at least 40 μg/L, such as at least 50 μg/L, such as at least 75 μg/L, such as at least 100 μg/L, such as at least 125 μg/L, such as at least 145 μg/L, such as at least 165 μg/L, such as at least 170 μg/L, such as at least 180 μg/L, such as at least 190 μg/L, such as at least 200 μg/L, such as at least 220 μg/L, such as at least 250 μg/L, such as at least 300 μg/L, such as at least 350 μg/L, such as at least 400 μg/L, such as at least 500 μg/L, such as at least 750 μg/L, such as at least 1 g/L, or more.

34. The method according to any one of 30 to 33, further comprising the steps of:
i. recovering the hemandulcin and/or the one or more derivatives thereof; and
ii. optionally converting said hemandulcin and/or the one or more derivatives thereof to one or more further derivatives thereof; and/or
iii. formulating said hemandulcin and/or the one or more derivatives thereof in a composition.

35. A composition, preferably a food composition and/or a beverage, comprising hemandulcin and/or one or more derivatives thereof obtained by the method according to any one of claims 30 to 34.

36. The composition according to claim 35, wherein the composition is a food composition and/or a beverage.

37. Hemandulcin and/or one or more derivatives thereof obtained by the method according to any one of claims 30 to 34.

38. Use of a Nicotiana cytochrome P450 enzyme, preferably a Nicotiana tabacum cytochrome P450 enzyme, in a method for producing hemandulcin and/or one or more derivatives, optionally wherein the Nicotiana tabacum cytochrome P450 enzyme is NtEAH as set forth in SEQ ID NO 2, or a functional variant thereof having at least 65% identity, homology or similarity thereto.

39. The use according to claim 38, wherein the polypeptide comprises the sequence as set forth in SEQ ID NO 2, with the exception that at the most 51 residues are mutated.

40. The use according to any one of claims 38 to 40, said use comprising expressing the Nicotiana cytochrome P450 enzyme in a yeast cell, preferably a Nicotiana tabacum cytochrome P450 enzyme, optionally wherein the Nicotiana tabacum cytochrome P450 enzyme is NtEAH as set forth in SEQ ID NO 2, or functional variants thereof having at least 65% identity, homology or similarity thereto, preferably wherein said yeast cell is as defined in any one of claims 1 to 22 or 29.

41. Use of a Datura cytochrome P450 enzyme, preferably a Datura stramonium cytochrome P450 enzyme, in a method for producing hemandulcin and/or one or more derivatives, optionally wherein the Datura stramonium cytochrome P450 enzyme is DsEAH as set forth in SEQ ID NO 11, or a functional variant thereof having at least 65% identity, homology or similarity thereto.

42. The use according to claim 41, wherein the polypeptide comprises the sequence as set forth in SEQ ID NO 11, with the exception that at the most 51 residues are mutated.

43. The use according to any one of claims 41 to 42, said use comprising expressing the Datura cytochrome P450 enzyme, preferably a Datura stramonium cytochrome P450 enzyme, optionally wherein the Datura stramonium cytochrome P450 enzyme is DsEAH as set forth in SEQ ID NO 11, or functional variants thereof having at least 65% identity, homology or similarity thereto in a yeast cell, preferably wherein said yeast cell is as defined in any one of claims 1 to 22 or 29.

44. Use of a Solanum cytochrome P450 enzyme, preferably a Solanum lycopersicum cytochrome P450 enzyme, in a method for producing hemandulcin and/or one or more derivatives, optionally wherein the Solanum lycopersicum cytochrome P450 enzyme is SIEAH as set forth in SEQ ID NO 9, or a functional variant thereof having at least 65% identity, homology or similarity thereto.

45. The use according to claim 44, wherein the polypeptide comprises the sequence as set forth in SEQ ID NO 9, with the exception that at the most 54 residues are mutated.

46. The use according to any one of claims 44 to 45, said use comprising expressing the Solanum cytochrome P450 enzyme in a yeast cell, preferably a Solanum lycopersicum cytochrome P450 enzyme, optionally wherein the Solanum lycopersicum cytochrome P450 enzyme is SIEAH as set forth in SEQ ID NO 9, or functional variants thereof having at least 65% identity, homology or similarity thereto, preferably wherein said yeast cell is as defined in any one of claims 1 to 22 or 29.

47. Use of a Capsicum cytochrome P450 enzyme, preferably a Capsicum chinense cytochrome P450 enzyme and/or a Capsicum annuum cytochrome P450 enzyme, in a method for producing hemandulcin and/or one or more derivatives, optionally wherein the Capsicum chinense cytochrome P450 enzyme is CcEAH as set forth in SEQ ID NO 13, the Capsicum annuum cytochrome P450 enzyme is CaEAH as set forth in SEQ ID NO 15, or functional variants thereof having at least 65% identity, homology or similarity to any of the aforementioned.

48. The use according to claim 47, wherein the polypeptide comprises the sequence as set forth in SEQ ID NO 13 and/or SEQ ID NO 15, with the exception that at the most 51 residues are mutated.

49. The use according to any one of claims 47 to 48, said use comprising expressing the Capsicum cytochrome P450 enzyme in a yeast cell, preferably a Capsicum chinense cytochrome P450 enzyme and/or the Capsicum annuum cytochrome P450 enzyme, optionally wherein the Capsicum chinense cytochrome P450 enzyme is CcEAH as set forth in SEQ ID NO 13, the Capsicum annuum cytochrome P450 enzyme is CaEAH as set forth in SEQ ID NO 15, or functional variants thereof having at least 65% identity, homology or similarity to any of the aforementioned, preferably wherein said yeast cell is as defined in any one of claims 1 to 22 or 29.

50. The use according to any one of claims 38 to 49, wherein said method is as defined in any one of claims 30 to 34.

51. A fermentation liquid comprising hemandulcin and/or one or more derivatives thereof, wherein said fermentation liquid is obtained by a method according to any one of claims 30 to 34, optionally wherein at least 50% of the yeast cells are disrupted and/or wherein at least 50% of cellular material, such as yeast cell debris, is separated from the fermentation liquid.


SEQUENCE LISTING (16 sequences)

SEQ ID NO: 1 - protein, 546 aa, Lippia dulcis
MNSTSRRSANYKPTIWNNEYLQSLNSIYGEKRFLEQAEKLKDEVRMLLEKTSDPLDHIELVDVLQRLAISYHFTEYIDRNLKNIYDILIDGRRWNHADNLHATTLSFRLLRQHGYQVSPEVFRNFMDETGNFKKNLCDDIKGLLSLYEASYLLTEGETIMDSAQAFATHHLKQKLEENMNKNLGDEIAHALELPLHWRVPKLDVRWSIDAYERRQDMNPLLLELAKLDFNIAQSMYQDELKELSRWYSKTHLPEKLAFARDRLVESYLWGLGLASEPHHKYCRMMVAQSTTLISIIDDIYDVYGTLDELQLFTHAVDRWDIKYLEQLPEYMQICFLALFNTVNERSYDFLLDKGFNVIPHSSYRWAELCKTYLIEANWYHSGYKPSLNEYLNQGLISVAGPHALSHTYLCMTDSLKEKHILDLRTNPPVIKWVSILVRLADDLGTSTDELKRGDNPKSIQCHMHDTGCNEEETRAYIKNLIGSTWKKINKDVLMNFEYSMDFRTAAMNGARVSQFMYQYDDDGHGVPEGKSKERVCSLIVEPIPLP

SEQ ID NO: 2 - protein, 504 aa, Nicotiana tabacum
MQFFSLVSIFLFLSFLFLLRKWKNSNSQSKKLPPGPWKIPILGSMLHMIGGEPHHVLRDLAKKYGPLMHLQLGEISAVVVTSRDMAKEVLKTHDVVFASRPKIVAMDIICYNQSDIAFSPYGDHWRQMRKICVMELLNAKNVRSFSSIRRDEVVRLIDSIRSDSSSGELVNFTQRIIWFASSMTCRSAFGQVLKGQDIFAKKIREVIGLAEGFDVVDIFPTYKFLHVLSGMKRKLLNAHLKVDAIVEDVINEHKKNLAAGKSNGALGGEDLIDVLLRLMNDTSLQFPITNDNIKAVIVDMFAAGTETSSTTTVWAMAEMMKNPSVFTKAQAEVREAFRDKVSFDENDVEELKYLKLVIKETLRLHPPSPLLVPRECREDTDINGYTIPAKTKVMVNVWALGRDPKYWDDAESFKPERFEQCSVDFFGNNFEFLPFGGGRRICPGMSFGLANLYLPLAQLLYHFDWKLPTGIMPRDLDLTELSGITIARKGGLYLNATPYQPSRE

SEQ ID NO: 3 - protein, 711 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 4 - DNA, 1641 bp, synthetic construct
  source 1..1641: mol_type = other DNA
atgaactccacctcccgacgatccgccaactataagcctactatttggaacaacgaatacctgcagtctctaaactctatttacggtgagaagcgattcctggagcaagctgaaaagctcaaggatgaggtgagaatgctgctcgagaagacctctgaccctctggaccacatcgaactggtggatgtcctgcagcgactggccatttcctaccacttcaccgagtatatcgaccgaaacctcaaaaacatctacgacatcctcatcgacggccgacgatggaaccacgctgacaacctgcacgccactaccctttccttccgcctcctgcgacagcacggttaccaggtctcccccgaggttttccgaaacttcatggacgagactggcaactttaagaagaatttgtgcgacgacatcaagggtctgttgtccctgtacgaggcatcttacctgcttactgaaggtgagaccatcatggactctgcccaggccttcgccactcatcacctgaagcagaagctggaggagaacatgaacaagaacctcggtgacgagattgctcacgctctcgagctgcccctgcactggcgagtccccaagcttgacgtcagatggtccatcgacgcctacgagcgaagacaggacatgaaccctctgctgctggaactggccaagctggatttcaatatcgcccagtctatgtaccaggacgaattgaaggagctgagccgatggtactccaagacccaccttcccgaaaaactcgcgttcgcccgagaccgactcgtcgagtcttacctttggggccttggtctggccagcgagccccaccacaagtactgtcgaatgatggtggcccagtccactactctgatctccattatcgacgacatctacgatgtttacggtaccctcgatgagctgcagctcttcacccatgccgtggaccgatgggacattaagtaccttgaacaactgcctgaatacatgcagatttgcttcctggccctgtttaataccgtgaacgagcgatcttacgacttcctgcttgacaagggctttaacgtcatcccccattcctcctaccgctgggctgagctctgcaagacttacctgatcgaggccaactggtaccactccggctacaaaccctcactgaacgagtacctgaaccagggactcatctccgttgctggcccccacgccctgtcccacacctacctgtgtatgactgactcgctgaaagagaagcacatcctggacctccgaactaacccgcccgtcatcaagtgggtctccatcctcgtccgcctggccgatgacctcggcacctccaccgacgaactgaaacgaggtgacaaccccaagagcatccagtgccacatgcatgataccggttgcaacgaggaggagactcgagcctatattaagaaccttatcggctctacctggaagaagattaataaggacgtcctcatgaattttgagtactctatggatttccgaaccgccgccatgaacggagcccgcgtctctcagttcatgtatcagtacgacgatgacggtcatggcgtgcccgagggcaagtccaaggagcgagtgtgctccctgatcgtcgagcccatccccctgccctag

SEQ ID NO: 5 - DNA, 1515 bp, synthetic construct
  source 1..1515: mol_type = other DNA
atgcagttcttctccctcgtctccatcttcctcttcctctccttcctcttcctcctccgaaagtggaagaactccaactcccagtccaagaagctcccccctggtccttggaagatccccatcctcggttccatgctccacatgatcggtggtgagccccaccacgtcctccgagatctcgccaagaagtacggtcccctcatgcacctccagctcggtgagatctccgccgtcgtcgtcacctcccgagacatggccaaggaggtcctcaagacccacgacgtcgtcttcgcctcccgacctaagatcgtcgccatggacatcatctgctacaaccagtccgacatcgccttctccccctacggtgaccactggcgacagatgcgaaagatctgcgtcatggagctcctcaacgccaagaacgtccgatccttctcctccatccgacgagacgaggtcgtccgactcatcgactccatccgatccgactcctcctccggtgagctcgtcaacttcacccagcgaatcatctggttcgcctcctccatgacctgccgatccgccttcggtcaggtcctcaagggtcaggacatcttcgccaagaagatccgagaggtcatcggtctggccgagggtttcgacgtcgtcgacatcttccccacctacaagttcctccacgtcctctccggtatgaagcgaaagctcctcaacgcccacctcaaggtcgacgccatcgtcgaggacgtcatcaacgagcacaagaagaacctcgccgctggtaagtccaacggtgccctcggtggtgaggacctcatcgacgtcctcctccgactcatgaacgacacctccctccagttccccatcaccaacgacaacatcaaggccgtcatcgtcgacatgttcgccgccggtactgagacttcctccaccaccaccgtctgggccatggccgagatgatgaagaacccctccgtcttcaccaaggcccaggctgaggtccgagaggccttccgagacaaggtttccttcgacgagaacgacgtcgaggagctcaagtacctcaagctcgtcatcaaggagactctccgactccaccccccttcccctctcctcgtcccccgagagtgccgagaggacaccgacatcaacggttacaccatccccgccaagaccaaggtcatggtcaacgtctgggccctcggtcgagatcccaagtactgggacgacgccgagtccttcaagcccgagcgattcgagcagtgctccgtcgacttcttcggtaacaacttcgagttcctccccttcggtggtggtcgacgaatctgccccggtatgtccttcggtctggccaacctctacctccccctcgctcagctcctctaccacttcgactggaagctccccaccggtatcatgccccgagatctcgacctcaccgagctctccggtatcaccatcgcccgaaagggtggtctgtacctcaacgccaccccctaccagccctcccgagagtaa

SEQ ID NO: 6 - DNA, 2136 bp, synthetic construct
  source 1..2136: mol_type = other DNA
atgtcctcttcgtcgtcctcttctacctctatgatcgacctgatggccgccatcatcaagggcgagcccgtgatcgtgtctgaccccgccaacgcctctgcctacgagtctgtggccgccgagctgtcctctatgctgatcgagaaccgacagttcgccatgatcgtgaccacctctatcgccgtgctgatcggctgcatcgtgatgctggtgtggcgacgatctggctctggcaactctaagcgagtcgagcccctgaagcccctggtgatcaagccccgagaggaggagatcgacgacggccgaaagaaggtgaccatcttcttcggcacccagaccggcactgccgagggcttcgccaaggccctgggcgaggaggccaaggcccgatacgagaagacccgattcaagatcgtggacctggacgactacgccgccgacgacgacgagtacgaggagaagctgaagaaggaggacgtcgccttcttcttcctggccacctacggcgacggcgagcccaccgacaacgccgctcgattctacaagtggttcaccgagggcaacgaccgaggcgagtggctgaagaacctgaagtacggcgtgttcggcctgggcaaccgacagtacgagcacttcaacaaggtggccaaggtggtggacgacatcctggtcgagcagggcgctcagcgactggtgcaggtcggcctgggcgacgacgaccagtgcatcgaggacgacttcaccgcctggcgagaggccctgtggcccgagctggacaccatcctgcgagaggagggcgacaccgccgtcgccaccccttacaccgccgctgtgctcgagtaccgagtgtctatccacgactctgaggacgccaagttcaacgacatcaacatggccaacggcaacggctacaccgtgttcgacgcccagcacccctacaaggccaacgtggccgtgaagcgagagctgcacacccccgagtctgaccgatcttgcatccacctcgagttcgacattgccggctctggcctgacctacgagactggcgaccacgtgggcgtgctgtgcgacaacctgtctgagactgtggacgaggccctgcgactgctggacatgtctcccgacacctacttctcgctgcacgccgagaaggaggatggaacccccatctcttcgtcgctgccccctcccttccccccctgcaacctgcgaaccgccctgacccgatacgcctgcctgctgtcctcgcccaagaagtctgccctggtcgccctggccgctcacgcttctgaccccaccgaggccgagcgactgaagcacctggcctctcccgccggaaaggacgagtactctaagtgggtggtcgagtctcagcgatctctgctcgaggtgatggccgagttcccctcggccaagccccctctgggcgtgttcttcgccggcgtggctccccgactgcagccccgattctactctatctcttcgtcccccaagattgccgagactcgaatccacgtgacctgcgccctggtgtacgagaagatgcccaccggccgaatccacaagggcgtgtgctctacctggatgaagaacgccgtgccctacgagaagtctgagaactgttcttctgcccccatcttcgtgcgacagtctaacttcaagctgccctctgactctaaggtgcccatcatcatgatcggacccggcaccggcctggctcccttcagaggcttcctgcaggagcgactggccctggtcgagtctggcgtcgagctgggcccctctgtgctgttcttcggctgccgaaaccgacgaatggacttcatctacgaggaggagctgcagcgattcgtcgagtctggtgccctggctgagctgtctgtggccttctcgcgagagggacccaccaaggagtacgtccagcacaagatgatggacaaggcctctgacatctggaacatgatctctcagggcgcctacctgtacgtgtgcggcgacgccaagggcatggcccgagatgtgcaccgatctctgcacaccattgcccaggagcagggctctatggactctaccaaggccgagggattcgtgaagaacctgcagacctctggccgatacctgcgagatgtgtggtga

SEQ ID NO: 7 - protein, 708 aa, Lippia dulcis
MDSTTEKLSPFDLMSAILKGVKLDNSNGSADATPPAVVAMLMENKDLMMMITTSMAVLIGCLVFLVWRKAAGSKKKVEPPRLVVPKAQEPEEVDDGKKKVTIFFGTQTGTAEGFAKALAEEAKARYQQAKFKVVDIDDYAADDEEYEEKFKKETVAFFFLATYGDGEPTDNAARFYKWFMEEQERGDWLKNMSYGVFGLGNRQYEHFNKIGIEVDKIIADGGGNRLIPVGLGDDDQCIEDDFTAWRESLWPELDKLLRDEDDSSAATPYTAAILEYRVVFHDQPETVSENGSANGYANGNTVYDSQHPYRANVAIKKELHTPASDRSCTHLEFDISGSGLSYETGDHVGVYCENLIETVEEAERLLNMPPQTYFSIHTDNEDGTPRDGSSLAPPFPPCTLRKALTLYADLLSGPKKSALIALATYASDPAEADRLKHLASPDGKEEYAQYIVASQRSLLEVMADFPSAKPPLGVFFAAVAPRLQARFYSISSSPKIAPNRIHVTCSLVYEKTPTGRIHKGVCSTWMKNALPSEDALDCSTAPIFVRTSNFRLPSDPEVPIIMIGPGTGLAPFRGFLQERSALKESGAELGPAVLFFGCRNSKMDYIYEDELNNFFKNGVISELVLAFSREGPTKEYVQHKMVQKASDLWKLISDGAYIYVCGDAKGMARDVHRTLHTIVQEQGSLDSSKTESFVKNLQMTGRYLRDVW

SEQ ID NO: 8 - DNA, 2127 bp, synthetic construct
  source 1..2127: mol_type = other DNA
atggactctaccaccgagaagctgtctcccttcgacctgatgtctgccatcctgaagggcgtgaagctggacaactctaacggctctgccgacgctacccctcctgccgtggtggccatgctgatggaaaacaaggacctcatgatgatgatcaccacctctatggccgtgctgatcggctgcctggtgttcctggtgtggcgaaaggccgctggctctaagaagaaggtcgagcctcctcgactggtggtgcccaaggctcaagagcccgaggaagtggacgacggcaagaagaaggttaccattttcttcggcacccagaccggcaccgccgagggcttcgccaaggctctggccgaggaagctaaggcccgataccagcaggccaagttcaaggtggtggacatcgacgactacgccgccgacgacgaggaatacgaggaaaagttcaagaaggaaaccgtcgctttcttcttcctggctacctacggcgacggcgagcccaccgacaacgccgctcgattctacaagtggttcatggaagaacaagagcgaggcgactggctgaagaacatgtcttacggcgtgttcggcctgggcaaccgacagtacgagcacttcaacaagatcggcatcgaggtggacaagatcattgccgacggcggaggtaaccgactgatccccgtcggactgggcgacgacgaccagtgcatcgaggacgacttcaccgcctggcgagagtctctgtggcccgagctggataagctgctgcgagatgaggacgattcttctgccgctactccctacaccgccgccatcctcgagtaccgagtggtgttccacgaccagcctgagactgtgtctgagaacggatctgccaacggctacgccaacggcaacaccgtgtacgactctcagcacccctaccgagccaacgtggccattaagaaggaactgcacacccctgcctctgaccgatcttgcacccacctcgagttcgacatctctggctctggactgtcttacgagactggcgaccacgtgggcgtgtactgcgagaacctgatcgagactgtggaagaggccgagcgactgctgaacatgcctcctcagacctacttctctatccacactgacaacgaggacggcacccctcgagatggatcttctctggcccctccttttcctccttgcactctgcgaaaggctctgaccctgtacgccgacctgctgtcgggccccaagaagtctgccctgatcgccctcgccacctacgcctctgatcccgccgaggctgaccgactgaagcacctggcttctcccgacggaaaggaagagtacgcccagtacatcgtggcctctcagcgatctctgctcgaggtgatggctgacttcccgtctgccaagcctcctctgggcgtgttcttcgccgccgtggctccccgactgcaggcccgattctactctatctcttcgtcccctaagatcgctcccaaccgaatccacgtgacctgttctctggtgtacgaaaagactcccaccggacgaatccacaagggcgtgtgctctacctggatgaagaacgctctgccctctgaggacgccctggactgctctaccgctcctatcttcgtgcgaacctctaacttccgactgccttctgaccccgaggtgcccatcattatgatcggacccggcaccggactggctcccttcagaggcttcctgcaagagcgatctgctctgaaggaatctggcgctgagctgggacccgccgtgctgttcttcggctgccgaaactctaagatggactacatctacgaggacgagctgaacaacttcttcaagaacggcgtgatctctgagctggtgctggccttttcgcgagagggccccaccaaggaatacgtgcagcacaagatggtgcaaaaggcctccgacctgtggaagctgatctccgacggcgcctacatctatgtgtgcggcgacgccaagggcatggcccgagatgtgcatcgaaccctgcacaccatcgtgcaagagcagggctctctggactcttctaagaccgagtctttcgtgaagaacctgcagatgaccggccgatacctgcgggacgtgtggtaa

SEQ ID NO: 9 - protein, 536 aa, Solanum lycopersicum
MDNSPPQQIKALSLRFERHASLSFSYLMEFQFPFNIIFLFLFLSFLYLLLVEWKKSRNLNKKLPPGPWKLPFLGSLHHLAIGGLPHHALTKLGKKYGPLMHLQLGEISTVVVSSMDMAREVLKTHDLAFASRPKLASIDIICYKSTDIVFSPYGEYWRQMRKVCVMELLTAKNVRSFSSILHDEASRLVQFIQSSTHGEPINITERILWYTSSITCKTAFGDQLLKDQEKFIQMVKKLVELASGFSLADIFPSIKILGVLTGARSRILKVHKNVDAIVEDVINEHKKNLASGKKGNGAFGGENLVDVLIRLMGSGELKIPITNDNIKAIMVDLFSAGTETSSTTATWAMTEMMRNPRVLKKAQAEVREAFKGKETFDEDVIEELKYLRQVVKETLRLHPPVPLLAPRECREETNINGYTIPLKTRVMVNVWSMGRDPKYWEDAESFIPERFARSSVDFLGNNFEYLPFGAGRRMCPGITFGLFNVYLPLAKLLYHFDWKLPDGQKPEDVDMTELSAITAARKSELYLIATPYHPSQ

SEQ ID NO: 10 - DNA, 1611 bp, synthetic construct
  source 1..1611: mol_type = other DNA
atggacaactcgcctcctcagcagatcaaggccctgtctctgcgattcgagcgacacgcctctctgtctttctcttacctgatggaatttcagttccccttcaacattatctttctgtttctgttcctgtcgttcctgtacctgctgctggtcgagtggaagaagtctcgaaacctgaacaagaagctgcctcctggaccttggaagctgcccttcctgggctctctgcaccacctggccatcggcggactgccccaccacgctctgaccaagctgggcaagaagtacggacccctgatgcacctccagctgggcgagatctctaccgtggtggtgtcctctatggacatggcccgagaggtgctcaagacccacgacctggccttcgcctctcgacccaagctggcctctatcgacatcatctgctacaagtctaccgacatcgtgttctcgccctacggcgagtactggcgacagatgcgaaaggtgtgcgtgatggaactgctgaccgccaagaacgtgcgatctttctcctctatcctgcacgacgaggcttctcgactggtgcagttcatccagtcgtctacccacggcgagcccatcaacatcaccgagcgaatcctgtggtacacctcttctatcacctgtaagaccgccttcggcgaccagctgctgaaggaccaagagaagttcattcagatggtgaagaagctggttgagctggcttctggcttctctctggccgacatcttcccatctatcaagatcctgggcgtgctgaccggcgctcgatcccgaatcctgaaggtgcacaagaacgtcgacgccatcgtcgaggacgtgatcaacgagcacaagaagaacctggcctctggcaagaagggcaacggcgccttcggaggcgagaacctggtggacgtgctgatccgactgatgggatctggcgagctgaagatccccatcaccaacgacaacatcaaggccatcatggtggacctgttctctgccggaaccgagacttcctctaccaccgccacctgggccatgaccgagatgatgcgaaaccctcgagtgctgaagaaggcccaggccgaggtccgagaggccttcaagggcaaggaaaccttcgacgaggacgtcatcgaggaactgaagtacctgcgacaggtggtgaaggaaaccctgcgactgcaccctcctgtgcctctgctcgctccccgagagtgccgagaggaaactaacatcaacggctacaccattcctctcaagactcgagtgatggtgaacgtgtggtctatgggacgagatcccaagtattgggaagatgccgagtctttcatccccgagcgattcgcccgatcttctgtggacttcctgggaaacaacttcgagtacctgcctttcggagccggccgacgaatgtgccccggcatcaccttcggcctgttcaacgtctacctgcctctcgctaagctgctgtaccacttcgactggaagctccccgacggacaaaagcctgaggacgtggacatgactgagctgtctgccatcaccgccgctcgaaagtctgagctgtacctgatcgctaccccttaccatccttctcagtaa

SEQ ID NO: 11 - protein, 506 aa, Datura stramonium
MEDLFDLVSISLFLSFLFLLIRKWKNNWNSQNKKLPPGPWKLPFIGSMHHLAGGLPHRVLGDLAKKYGPLMHLQLGEVSAVVVSSSEMAKQVLKTHDLAFATRPRLLATDIIFYDRLDIAFSPYGEYWRQMRKICVLELLSAKNVRSFSSIRQGEVVHLVDSIRSSSSSGELVNFTERMIWFTSSMTCRSAFGQVLKEQDMFIKEIRDVIGLAEGFDVADIFPSYKFLHGLSGAKKRILDAHHKVDSIVEDVINEHKKNLETHKSTDDDALGGEDLIDVLLRLMKDGSLQFPITNNNIKAVIIDMFAAGTETSSTTIVWAMVEMMKNPNIFAKAQEEVREAFKDKVTFDTNDVEELRYLKLVIKETLRLHPPLPLLAPRECREETDINGYTIPVKTKVMINVWALGRDPKYWDDAESFKPERFEQFSMDFVDNNFEYLPFGGGRRVCPGISFGLTNVYLPLAQLLYHFDWELPTGMKPEDLDLIELAGITSARKNDLYLMATPYQP

SEQ ID NO: 12 - DNA, 1521 bp, synthetic construct
  source 1..1521: mol_type = other DNA
atggaagatctgttcgacctggtgtctatctctctgttcctgtctttcctgttcctgctgatccgaaagtggaagaacaactggaactctcagaacaagaagctgcctcctggaccttggaagctgcccttcatcggctctatgcaccacctggctggcggactgccccaccgagtgctgggcgacctggccaagaagtacggacccctgatgcacctccagctcggcgaggtgtctgccgtggtggtgtcctcttcggagatggccaagcaggtcctcaagacccacgacctggctttcgccactcgaccccgactgctggccaccgacatcatcttctacgaccgactggacattgccttctcgccctacggcgagtactggcgacagatgcgaaagatctgcgtgctcgagctgctgtctgccaagaacgtgcgatctttctcgtctatccgacagggcgaagtggtgcacctggtggactctatccgatcttcttcgtcctctggcgagctggtgaacttcaccgagcgaatgatctggttcacctcttctatgacctgccgatctgccttcggccaggtgctgaaggaacaagacatgttcatcaaggaaatccgggacgtcatcggcctggccgagggcttcgacgtggccgacatcttcccgtcttacaagttcctgcacggcctgtctggcgccaagaagcgaatcctggacgcccaccacaaggtggactccatcgtcgaggacgtgatcaacgagcacaagaagaacctcgagactcacaagtctaccgacgacgacgccctcggcggcgaggacctgatcgacgtgctgctgcgactgatgaaggacggctctctgcagttccccatcaccaacaacaacatcaaggccgtgatcatcgacatgttcgccgctggaaccgagacttcttctaccaccatcgtgtgggccatggtcgagatgatgaagaaccccaacatcttcgccaaggctcaagaggaagtgcgagaggccttcaaggacaaggtgaccttcgacaccaacgacgtcgaggaactgcgatacctgaagctggtgattaaggaaaccctgcgactgcaccctcctctgcctctgctggctccccgagagtgccgagaggaaaccgacattaacggctacaccattcctgtcaagaccaaggtgatgatcaacgtctgggccctgggacgagatcccaagtactgggacgacgccgagtctttcaagcccgagcgattcgagcagttctctatggacttcgtggacaacaacttcgagtacctgcctttcggcggaggccgacgagtgtgccccggcatctctttcggcctgaccaacgtctacctgcctctggctcagctgctctaccacttcgactgggagctgcccaccggcatgaagcccgaggacctcgacctgattgagctggccggcatcacctctgctcgaaagaacgacctgtacctgatggctaccccttaccagccttaa

SEQ ID NO: 13 - protein, 502 aa, Capsicum chinense
MQFFSLASIFLFLFSLFLLRKWKNSNSQTKKLPPGPWKLPFIGSMHHMAGGLPHRVLRDLAKKYGPLMHLQLGEVSAVVVTSSELAKQVLKTHDLAFASRPKLSAMDIICYDSRDIVFSPYGEYWRQMRKICVMELLSAKNVRSFSSIRHEEVNRLIESIRSSSSSSELINFTERVIWFTSCMICRSAFGQVLKEQDEFIKLIGEVVSLAGGFDVADIFPSYKFLHSLSPAKQTLLDAHRKVDAIVEDDVINEHKKNLENHNIDDAFGGEDLVDVLLRLMKDKSLQFPITNNNIKAIIVDMFAAGTETSSTTTVWAMVEMMKNPSILAKAQEEVREAFKDKATFDKVDVEELKYLKLVVKETMRFHAPVPLLVPRECREETNINGYTIPVKTKVMVNVWALGRDPKYWDDAESFKPERFEKCSMDFVGNNFEYLPFGSGRRMCPGMSFGLANVYLPLAQLLYHFDWKLPTGIEPRALDVTESSGATATRKNDLCLIATPYQP

SEQ ID NO: 14 - DNA, 1509 bp, synthetic construct
  source 1..1509: mol_type = other DNA
atgcagttcttctctctggcctctatcttcctgttcctgttctctctgttcctgctgcgaaagtggaagaactctaactctcagaccaagaagctgcctcctggaccttggaagctgcccttcatcggctctatgcaccacatggctggcggactgccccaccgagtgctgcgagatctggccaagaagtacggacccctgatgcacctccagctcggcgaggtgtctgccgtggtggtgacctcttctgagctggccaagcaggtcctcaagacccacgacctggccttcgcctctcgacccaagctgtctgccatggacatcatctgctacgactctcgagacatcgtgttctcgccctacggcgagtactggcgacagatgcgaaagatctgcgtgatggaactgctgtccgccaagaacgtgcgatctttctcttctatccgacacgaagaggtgaaccgactgatcgagtctatccgatcttcttcgtcgtcctctgagctgatcaacttcaccgagcgagtgatctggtttacctcttgcatgatctgccgatctgccttcggccaggtgctgaaggaacaagacgagttcatcaagctgatcggagaggtggtgtctctggccggtggcttcgacgtggccgacatcttcccgtcttacaagttcctgcactctctgtctcccgccaagcagaccctgctggacgcccaccgaaaggtggacgccatcgtcgaggacgatgtgatcaacgagcacaagaagaacctcgagaaccacaacatcgacgacgccttcggcggcgaggacctggtggacgtgctgctgcgactgatgaaggacaagtctctgcagttccccatcaccaacaacaacatcaaggccatcatcgtggacatgttcgccgctggaaccgagacttcttctaccaccaccgtgtgggccatggtcgagatgatgaagaacccctctatcctggccaaggctcaagaggaagtgcgagaggccttcaaggacaaggccaccttcgacaaggtcgacgttgaggaactgaagtacctgaagctggtggtgaaggaaaccatgcgatttcacgctcccgtgcctctgctggtgccccgagagtgccgagaggaaactaacatcaacggctacaccattcctgtcaagaccaaggtgatggtgaacgtctgggccctgggacgagatcccaagtactgggacgacgccgagtctttcaagcccgagcgattcgagaagtgctctatggacttcgtgggcaacaacttcgagtacctgcctttcggctctggccgacgaatgtgccccggcatgtctttcggactggccaacgtctacctgcctctggctcagctgctgtaccacttcgactggaagctccccaccggcatcgagccccgagcactggacgtgaccgagtcctctggcgccaccgccactcgaaagaacgacctgtgcctgatcgctaccccttaccagccttaa

SEQ ID NO: 15 - protein, 501 aa, Capsicum annuum
MQFFSLASIFLFLSFLFLVRKWKNSNSQTKKLPPGPWKLPFIGSMHHMAGGVPHRVLRDLCKKYGPLMHLQLGEVSVVVVTSSEMAKQVLKTHDLAFASRPRLLAFDIICYDRKDIAFSPYGEYWRQMRKICIMEVLSAKNVRSFSSIRHDEVVRLIDSIQSSSSLGDLVNFTERIIRFTSSMTCRSAFGQVLKEQDIFFKQISEVLSLAGGSDVADIFPSYKFLHSLSGAKQKLLDSHRKLDSIVENVINEHKKNLSTHRSDDALGGEDLIDVLLRLKKDGALQFSITNDNIKAVIIDMFTAGTETSSATIVWAMVEMLKNPRTFTKAQAEVRDAFRDKVTFDTNDVDELKYLKLVIKEALRLHPPVPLLVPRECREETCINGYTIPVKTKVMVNVWALGRDPKYWDDAESFIPERFEQCSVDFLGNNFEYLPFGSGRRICPGISFGLANVYLPLAQLLYHFDWKLPTGIEPRDLDLTESVGIAAGRKSDLYLIATPYQP

SEQ ID NO: 16 - DNA, 1506 bp, synthetic construct
  source 1..1506: mol_type = other DNA
atgcagttcttctctctggcctctatcttcctgttcctgtctttcctgtttctggtgcgaaagtggaagaactctaactctcagaccaagaagctgcctcctggaccttggaagctgcccttcatcggctctatgcaccacatggctggcggcgtgccccaccgagtgctgcgagatctgtgcaagaagtacggacccctgatgcacctccagctcggcgaggtgtctgtggtggtggtgacctcttctgagatggccaagcaggtcctcaagacccacgacctggccttcgcctctcgaccccgactgctggctttcgacatcatctgctacgaccgaaaggacattgccttctcgccctacggcgagtactggcgacagatgcgaaagatctgcatcatggaagtgctgtctgccaagaacgtgcgatctttctcttctatccgacacgacgaggtggtgcgactgatcgactctatccagtcctcttcttctctgggcgacctggtgaacttcaccgagcgaatcatccgattcacctcttctatgacctgccgatctgccttcggccaggtgctgaaggaacaagacatcttcttcaagcagatctctgaggtgctgtctctggccggtggctctgacgtggccgacatcttcccgtcttacaagttcctgcactctctgtctggcgccaagcagaagctgctggactctcaccgaaagctggactccatcgtcgagaacgtgatcaacgagcacaagaagaacctgtctacccaccgatctgacgacgccctcggcggcgaggacctgatcgacgtgctgctgcgactgaagaaggacggcgccctgcagttctctatcaccaacgacaacatcaaggccgtgatcatcgacatgttcaccgccggaaccgagacttcttctgccaccatcgtgtgggccatggtcgagatgctgaagaaccctcgaaccttcaccaaggctcaggccgaggtgcgagatgccttccgagacaaggtgaccttcgacactaacgacgtggacgagctgaagtacctgaagctggtgatcaaggaagccctgcgactgcaccctcctgtgcctctgctggtgccccgagagtgccgagaggaaacctgcatcaacggctacaccattcctgtcaagaccaaggtgatggtgaacgtctgggccctgggacgagatcccaagtactgggacgacgccgagtctttcatccccgagcgattcgagcagtgctctgtggacttcctgggcaacaacttcgagtacctgcctttcggctctggccgacgaatctgccccggcatctctttcggactggccaacgtctacctgcctctggctcagctgctgtaccacttcgactggaagctccccaccggcatcgagccccgagatctggacctgaccgagtctgtgggcattgccgccggacgaaagtctgacctgtacctgatcgctaccccttaccagccttaa